(19) United States
(12) Patent Application Publication
(10) Pub. No.: US 2011/0263529 A1
(43) Pub. Date: Oct. 27, 2011

(54) GAMMA SECRETASE MODULATORS

(76) Inventors: Ruo Xu, Watchung, NJ (US); John W. Clader, Milton, VT (US)

(21) Appl. No.: 13/125,995

(22) PCT Filed: Nov. 5, 2009
(86) PCT No.: PCT/US2009/063380

§ 371 Date: Jul. 18, 2011

Related U.S. Application Data
(60) Provisional application No. 61/111,823, filed on Nov. 6, 2008.

(51) Int. Cl. A61K 31/695; A61P 25/28; A61K 31/437; C07F 7/10; C07D 498/04

(52) U.S. Cl. 514/63; 546/14; 546/115; 514/302

(57) ABSTRACT

In its many embodiments, the present invention provides a novel class of heterocyclic compounds as modulators of gamma secretase, methods of preparing such compounds, pharmaceutical compositions containing one or more such compounds, methods of preparing pharmaceutical formulations comprising one or more such compounds, and methods of treatment, prevention, inhibition, or amelioration of one or more diseases associated with the central nervous system using such compounds or pharmaceutical compositions.

### REFERENCE TO RELATED APPLICATION

This application claims the benefit of U.S. Provisional Application No. 61/111,823 filed Nov. 6, 2008.

### FIELD OF THE INVENTION

The present invention relates to certain heterocyclic compounds useful as gamma secretase modulators (including inhibitors, antagonists and the like), pharmaceutical compositions containing the compounds, and methods of treatment using the compounds and compositions to treat various diseases including central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

### BACKGROUND OF THE INVENTION

Alzheimer's disease is a disease characterized by degeneration and loss of neurons and also by the formation of senile plaques and neurofibrillary change. Presently, treatment of Alzheimer's disease is limited to symptomatic therapies with a symptom-improving agent represented by an acetylcholinesterase inhibitor, and the basic remedy which prevents progress of the disease has not been developed. A method of controlling the cause of onset of pathologic conditions needs to be developed for creation of the basic remedy of Alzheimer's disease.

Aβ protein, which is a metabolite of amyloid precursor protein (hereinafter referred to as APP), is considered to be greatly involved in degeneration and loss of neurons as well as onset of demential conditions (for example, see Klein W L, et al Proceeding National Academy of Science USA, Sep. 2, 2003, 100(18), p. 10417-22, suggest a molecular basis for reversible memory loss.

Nitsch R M, and 16 others, Antibodies against β-amyloid slow cognitive decline in Alzheimer's disease, Neuron, May 22, 2003, 38(4), p. 547-554) suggest that the main components of Aβ protein are Aβ40 consisting of 40 amino acids and Aβ42 having two additional amino acids at the C-terminal. The Aβ40 and Aβ42 tend to aggregate (for example, see Jarrell J T et al, The carboxy terminus of the β amyloid protein is critical for the seeding of amyloid formation: implications for the pathogenesis of Alzheimer's disease, Biochemistry, May 11, 1993, 32(18), p. 4693-4697) and constitute main components of senile plaques (for example, (Glenner G G, et al, Alzheimer's disease: initial report of the purification and characterization of a novel cerebrovascular amyloid protein, Biochemical and Biophysical Research Communications, May 16, 1984, 120(3), p. 885-90. See also Masters C L, et al, Amyloid plaque core protein in Alzheimer disease and Down syndrome, Proceeding National Academy of Science USA, June 1985, 82(12), p. 4245-4249.).

Furthermore, it is known that mutations of APP and presenelin genes, which is observed in familial Alzheimer's disease, increase production of Aβ40 and Aβ42 (for example, see Gouras G K, et al, Intraneuronal Aβ142 accumulation in human brain, American Journal of Pathology, January 2000, 156(1), p. 15-20. Also, see Scheuner D, et al, Nature Medicine, August 1996, 2(8), p. 864-870; and Forman M S, et al, Differential effects of the Swedish mutant amyloid precursor protein on β-amyloid accumulation and secretion in neurons and nonneuronal cells, Journal of Biological Chemistry, Dec. 19, 1997, 272(51), p. 32247-32253.). Therefore, compounds which reduce production of Aβ40 and Aβ42 are expected as an agent for controlling progress of Alzheimer's disease or for preventing the disease.

These Aβs are produced when APP is cleaved by beta secretase and subsequently clipped by gamma secretase. In consideration of this, creation of inhibitors of γ secretase and β secretase has been attempted for the purpose of reducing production of Aβs. Many of these secretase inhibitors already known are peptides or peptidomimetics such as L-685,458. L-685,458, an aspartyl protease transition stale mimic, is a potent inhibitor of amyloid β-protein precursor γ-secretase activity, Biochemistry, Aug. 1, 2000, 39(30), p. 8698-8704).

Also of interest in connection with the present invention are: US 2006/0004013 (Eisai, published Jan. 5, 2006); WO 2005/110422 (Boehringer Ingelheim, published Nov. 24, 2005); WO 2006/045554 (CellZome AG, published May 4, 2006); WO 2004/110350 (Neurogenetics, published Dec. 23, 2004); WO 2004/071431 (Myriad Genetics, published Aug. 26, 2004); US 2005/0042284 (Myriad Genetics, published Feb. 23, 2005) and WO 2006/001877 (Myriad Genetics, published Jan. 5, 2006).

There is a need for new compounds, formulations, treatments and therapies to treat diseases and disorders associated with Aβ. It is, therefore, an object of this invention to provide compounds useful in the treatment or prevention or amelioration of such diseases and disorders.

### SUMMARY OF THE INVENTION

In its many embodiments, the present invention provides a novel class of heterocyclic compounds as gamma secretase modulators (including inhibitors, antagonists and the like), methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with the Aβ using such compounds or pharmaceutical compositions.

One embodiment, of the present invention is directed to compounds of formula I:

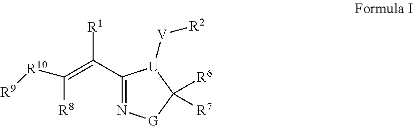

or a pharmaceutically acceptable salt, solvate, ester or prodrug thereof, wherein U, G, V, R¹, R², R⁶, R⁷, R⁸, R⁹, and R¹⁰ are as defined below.

This invention also provides compounds of formula I.

The present invention further includes the compound of formula I in all its isolated forms.

This invention also provides compounds of formula I in pure and isolated form.

This invention also provides compounds of formula I selected from the group consisting of: compounds of formulas II, III, IV, V (e.g., VA and VB), VI, VII, VIII, IX, and X.

This invention also provides compounds of formula I selected from the group consisting of: compounds B7, C1 (e.g., Enantiomer A of C1 and Enantiomer B of C1), and D1 to D12.

This invention also provides pharmaceutical compositions comprising an effective amount of one or more (e.g., one) compounds of formula I, or a pharmaceutically acceptable salt, ester or solvate thereof, and a pharmaceutically acceptable carrier.

This invention also provides pharmaceutical compositions comprising an effective amount of one or more (e.g., one) compounds of formula I, or a pharmaceutically acceptable salt, ester or solvate thereof, and an effective amount of one or more (e.g., one) other pharmaceutically active ingredients (e.g., drugs), and a pharmaceutically acceptable carrier.

The compounds of Formula I can be useful as gamma secretase modulators and can be useful in the treatment and prevention of diseases such as, for example, central nervous system disorders such as Alzheimers disease and Downs Syndrome.

Thus, this invention also provides methods for: (1) method for modulating (including inhibiting, antagonizing and the like) gamma-secretase; (2) treating one or more neurodegenerative diseases; (3) inhibiting the deposition of amyloid protein (e.g., amyloid beta protein) in, on or around neurological tissue (e.g., the brain); (4) Alzheimer's disease; and (5) treating Downs syndrome; wherein each method comprises administering an effective amount of one or more (e.g., one) compounds of formula I to a patient in need of such treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula I and the administration of an effective amount of one or more (e.g., one) other pharmaceutical active ingredients (e.g., drugs).

This invention also provides methods for: (1) treating mild cognitive impairment; (2) treating glaucoma; (3) treating cerebral amyloid angiopathy; (4) treating stroke; (5) treating dementia; (6) treating microgliosis; (7) treating brain inflammation; and (8) treating olfactory function loss; wherein wherein each method comprises administering an effective amount of one or more (e.g., one) compounds of formula I to a patient in need of such treatment.

This invention also provides a kit comprising, in separate containers, in a single package, pharmaceutical compositions for use in combination, wherein one container comprises an effective amount of a compound of formula I in a pharmaceutically acceptable carrier, and another container (i.e., a second container) comprises an effective amount of another pharmaceutically active ingredient (as described below), the combined quantities of the compound of formula I and the other pharmaceutically active ingredient being effective to treat the diseases or conditions mentioned in any of the above methods.

This invention also provides any of the above mentioned methods, pharmaceutical compositions or kit wherein the compound of formula I is selected from the group consisting of: compounds of formulas II, III, IV, V (e.g., VA and VB), VI, VII, VIII, IX, and X.

This invention also provides any of the above mentioned methods, pharmaceutical compositions or kit wherein the compound of formula I is selected from the group consisting of: compounds B7, C1 (e.g., Enantiomer A of C1 and Enantiomer B of C1), and D1 to D12.

### DETAILED DESCRIPTION

In one embodiment, the present invention discloses compounds which are represented by structural Formula I, or a pharmaceutically acceptable salt, solvate, ester or prodrug thereof.

Thus, one embodiment is directed to a compound of the formula I:

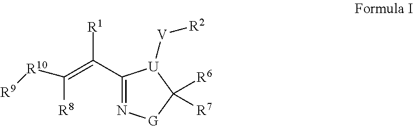

or a pharmaceutically acceptable salt, solvate, or ester thereof, wherein:

U is

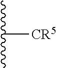

### or N;

G is O or S;

V is selected from the group consisting of a bond, O, —C(O)—, and N(R¹⁴);

R¹ is selected from the group consisting of: H, halo, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 substituents independently selected from the group consisting of the R²¹ groups;

R² is selected from the group consisting of: H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 substituents independently selected from the group consisting of the R²¹ groups; or

R¹ and R² are joined together to form a C5-C8 cycloalkyl or a 5-8 membered heterocyclyl moiety, wherein each of said cycloalkyl or heterocyclyl moiety is optionally substituted with 1-5 substituents independently selected from the group consisting of the R²¹ groups; or

R¹ and R⁸ are taken together to form a bond (i.e., there is a triple bond between the carbon atom to which R¹ was bonded to and the carbon to which R⁸ was bonded to, i.e., the compound of formula I is a compound of formula II:

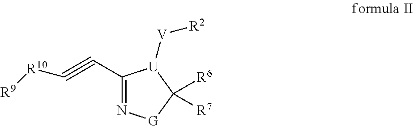

wherein G, U, V, R², R⁶, R⁷, R⁹, and R¹⁰ are as defined for formula I);

R⁵, R⁶ and R⁷ are each independently selected from the group consisting of H, —C(O)R¹⁵, —C(O)OR¹⁵, —C(O)N(R¹⁵)(R¹⁶), —C(═NOR¹⁵)R¹⁶, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, and wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 substituents independently selected from the group consisting of the R²¹ groups; or

R⁶ and R⁷ are joined together to form a carbocyclic spirocyclic moiety or a heterocyclic spirocyclic moiety wherein each of said carbocyclic spirocyclic moiety and heterocyclic spirocyclic moiety is: (i) optionally substituted with 1-4 substituents independently selected from the group consisting of the the R²¹ groups, or (ii) fused with an aryl, heteroaryl, cycloalkyl or heterocycloalkyl ring, and wherein each of said carbocyclic spirocyclic moiety, heterocyclic spirocyclic moiety, aryl, heteroaryl, cycloalkyl and heterocycloalkyl ring is optionally substituted with 1-4 substituents independently selected from the group consisting of the the R²¹ groups;

R⁸ is selected from the group consisting of H, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, —N₃, —NO₂ and —S(O)₂R²⁴, alkyl-, aryl-, arylalkyl-, alkylaryl-, cycloalkyl-, cycloalkylalkyl-, heteroaryl-, heteroarylalkyl-, heterocyclyl- and heterocyclyalkyl-, and wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-moieties is optionally substituted with 1-3 substituents independently selected from the group consisting of the the R²¹ groups;

R¹⁰ is selected from the group consisting of a bond, alkyl-, aryl-, arylalkyl-, arylalkenyl-, alkylaryl-, cycloalkyl-, cycloalkylalkyl-, heteroaryl-, heteroarylalkyl-, heterocyclyl-, heterocyclyalkyl- and the moieties:

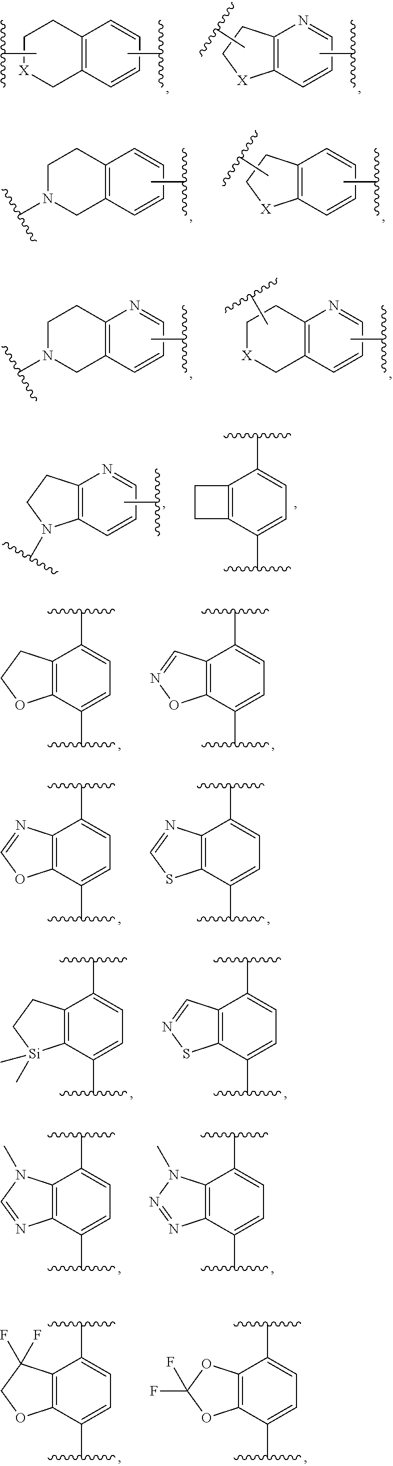

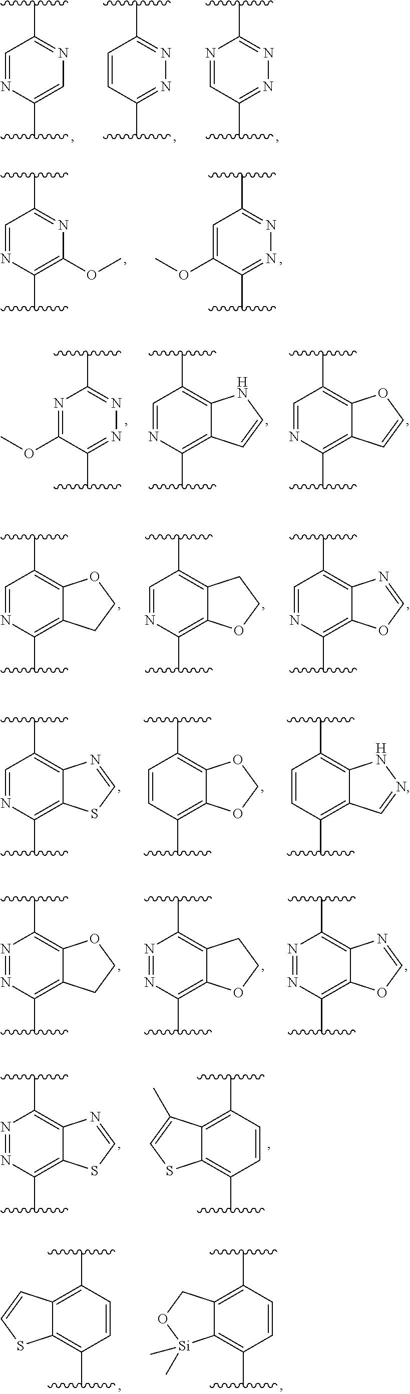

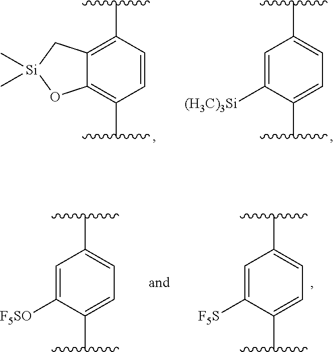

wherein X is selected from the group consisting of: O, N(R¹⁴) or S; and wherein each of said R¹⁰ groups (excluding the bond) is optionally substituted with 1-3 substituents independently selected from the group consisting of the R²¹ groups;

or, alternatively, R⁸ and R¹⁰, together with the carbon atom to which they are bound, can form a C₄-C₇ carbocyclic (e.g., cycloalkyl) ring, or a 4-7 membered heterocyclyl ring, or a C₄-C₇ carbocyclenyl (e.g., cycloalkenyl) ring, or a 4-7 membered heterocyclenyl ring; and wherein said carbocyclic ring, heterocyclyl ring, carbocyclenyl ring, or heterocyclenyl ring is optionally substituted with 1-5 independently selected R²¹ substituents;

R⁹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, and wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-3 substituents independently selected from the group consisting of the R²¹ groups;

R¹⁴ is selected from the group consisting of H, alkyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), and wherein each of the alkyl, cycloalkyl, cycloalkylalkyl-, cycloalkenyl, heterocyclyl, heterocyclylalkyl-, aryl, arylalkyl-, heteroaryl, heteroarylalkyl-groups is optionally substituted with 1-5 independently selected R²¹ groups;

R¹⁵, R¹⁶ and R¹⁷ are independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ₙ-alkyl-, (R¹⁸)ₙ-cycloalkyl-, (R¹⁸)ₙ-cycloalkylalkyl-, (R¹⁸)ₙ-heterocyclyl-, (R¹⁸)ₙ-heterocyclylalkyl-, (R¹⁸)ₙ-aryl-, (R¹⁸)ₙ-arylalkyl-, (R¹⁸)ₙ-heteroaryl- and (R¹⁸)ₙ-heteroarylalkyl-;

n is 1 to 5;

Each R¹⁸ is independently selected from the group consisting of: alkyl, alkenyl, alkynyl, aryl, arylalkyl-, arylalkenyl-, arylalkynyl-, —NO₂, halo, heteroaryl, —CF₃, —CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, \ —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl); or

two R¹⁸ moieties on adjacent carbons can be linked together to form:

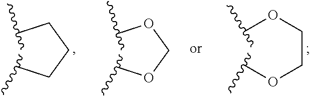

R¹⁹ is selected from the group consisting of: alkyl, cycloalkyl, aryl, arylalkyl and heteroarylalkyl;

R²⁰ is selected from the group consisting of: alkyl, cycloalkyl, aryl, halo substituted aryl, arylalkyl, heteroaryl and heteroarylalkyl;

Each R²¹ is independently selected from the group consisting of: alkyl, alkenyl, alkynyl, cycloalkyl, cycloalkylalkyl-, cycloalkenyl, heterocycloalkyl, heterocycloalkylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SF₅, —OSF₅, —Si(R²⁴)₃ wherein each R²⁴ is independently selected —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, —N₃, —NO₂ and —S(O)₂R²⁴;

wherein each of the alkyl, cycloalkenyl, cycloalkyl, cycloalkylalkyl-, heterocycloalkyl, heterocycloalkylalkyl-, aryl, arylalkyl-, heteroaryl, heteroarylalkyl-, alkenyl and alkynyl R²¹ groups is optionally substituted with 1 to 5 independently selected R²² groups;

Each R²² is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R²⁴)₃ wherein each R²⁴ is independently selected —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, —NO₂, —S(O)R¹⁵ and —S(O)₂R²⁴; and

Each R²⁴ is independently selected from the group consisting of alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ₙ-alkyl-, (R¹⁸)ₙ-cycloalkyl-, (R¹⁸)ₙ-cycloalkylalkyl-, (R¹⁸)ₙ-heterocyclyl-, (R¹⁸)ₙ-heterocyclylalkyl-, (R¹⁸)ₙ-aryl-, (R¹⁸)ₙ-arylalkyl-, (R¹⁸)ₙ-heteroaryl- and (R¹⁸)ₙ-heteroarylalkyl- (wherein R¹⁸ and n are as defined above); and

with the proviso that:
(a) there is present at least one (e.g., 1 to 3, or 1-2, or 1) group selected from the group consisting of: —SF₅, —OSF₅, and —Si(R²⁴)₃ (wherein each R²⁴ is independently selected), and wherein there is more than one group, each group is independently selected, or
(b) there is present an R¹⁰ group selected from the group consisting of:

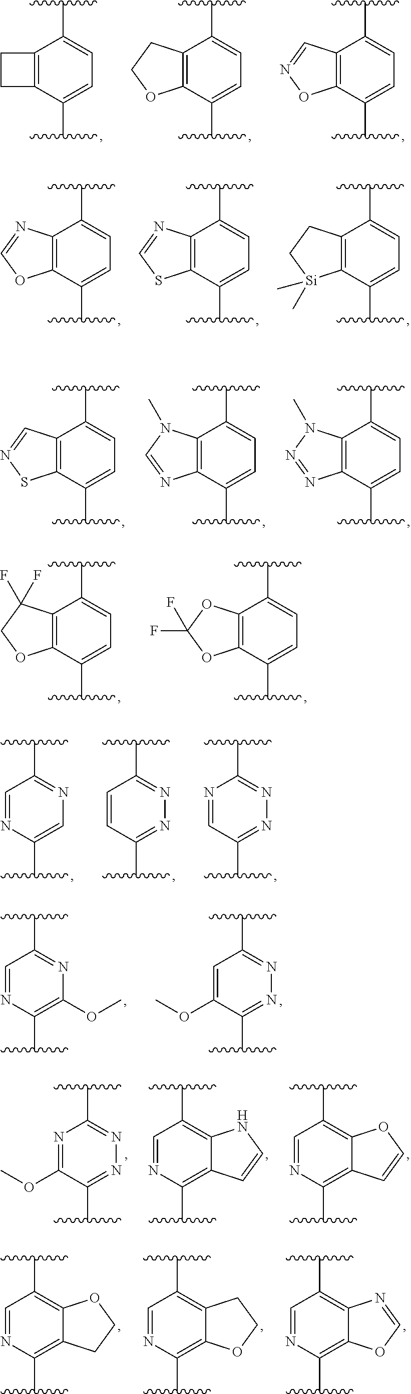

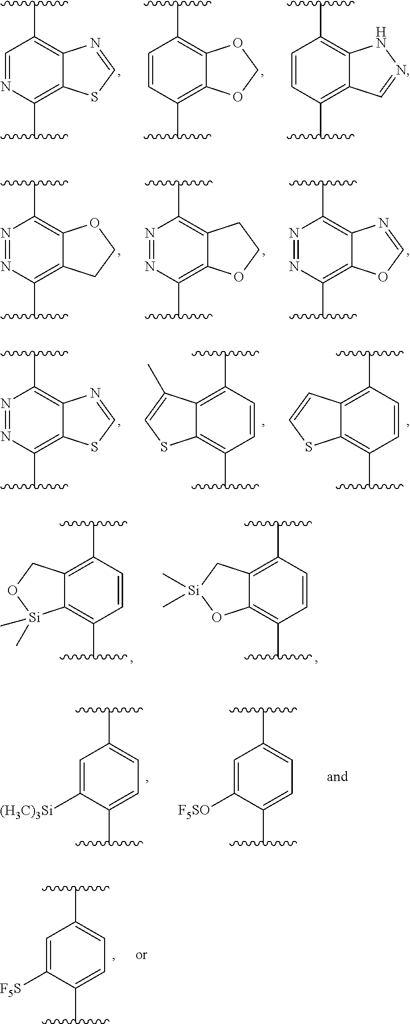

(c) there is present at least one (e.g., 1 to 3, or 1-2, or 1) group selected from the group consisting of: —SF₅, —OSF₅, —Si(R²⁴)₃ (wherein each R²⁴ is independently selected), and wherein there is more than one group, each group is independently selected, and there is present an R¹⁰ group selected from the group consisting of:

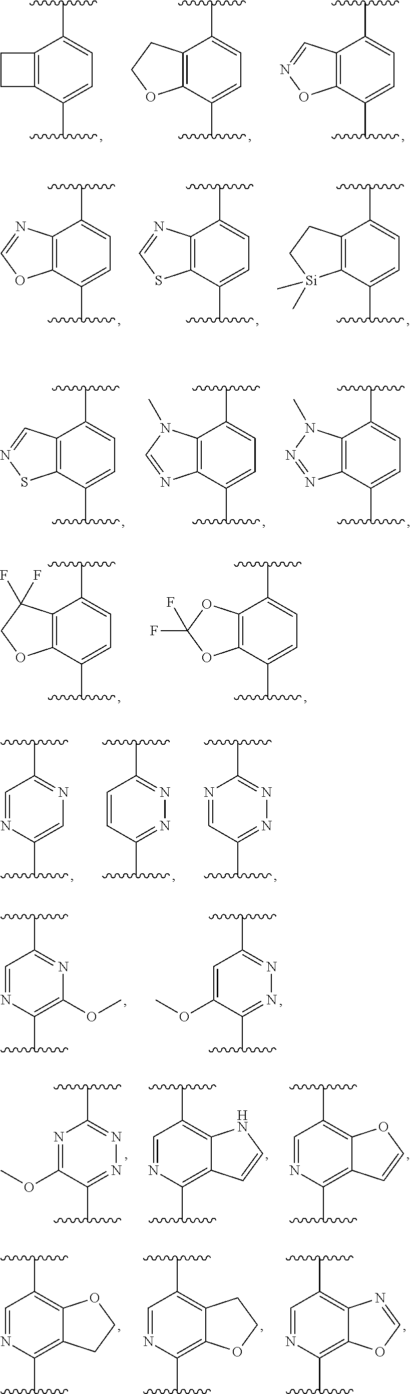

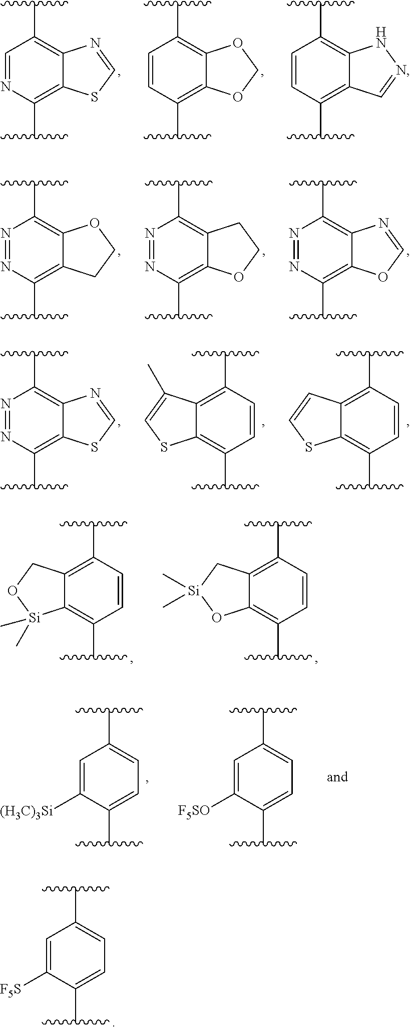

Those skilled in the art will appreciate that the —SF₅, —OSF₅, and —Si(R²⁴)₃ groups are present in the compounds of formula I: (a) due to the presence of at least one R²¹ group that is selected from the group consisting of: —SF₅, —OSF₅, and —Si(R²⁴)₃, or (b) due to the presence of at least one R²² substituent on at least one R²¹ group, wherein said R²² substituent is selected from the group consisting of: —SF₅, —OSF₅, and —Si(R²⁴)₃, and wherein said R²¹ group is other than a —SF₅, —OSF₅, or —Si(R²⁴)₃ group.

Another embodiment of this invention is directed to a compound of formula I wherein:
(a) U, G, V, R¹, R², R⁵, R⁶, R⁷, R⁸, R⁹, R¹⁰, R¹⁴, R¹⁵, R¹⁶, R¹⁷, n, R¹⁸, R¹⁹, R²⁰, R²¹, R²², and R²⁴ are as defined above for formula I, and
(b) with the proviso that there is present at least one (e.g., 1 to 3, or 1-2, or 1) group selected from the group consisting of: —SF₅, —OSF₅, and —Si(R²⁴)₃ (wherein each R²⁴ is independently selected), and when there is more than one group, each group is independently selected.
   Thus, this embodiment is directed to compounds of formula I wherein there is present at least one (e.g., 1 to 3, or 1-2, or 1) group selected from the group consisting of: —SF₅, —OSF₅, and —Si(R²⁴)₃ (wherein each R²⁴ is independently selected), and when there is more than one group, each group is independently selected.

Another embodiment of this invention is directed to a compound of formula I wherein:
(a) U, G, V, R¹, R², R⁵, R⁶, R⁷, R⁸, R⁹, R¹⁰, R¹⁴, R¹⁵, R¹⁶, R¹⁷, n, R¹⁸, R¹⁹, R²⁰, R²¹, R²², and R²⁴ are as defined above for formula I, and
(b) with the proviso that there is present at least one (e.g., 1 to 3, or 1-2, or 1) group selected from the group consisting of: —SF₅ and —OSF₅, and when there is more than one group, each group is independently selected. Thus, this embodiment is directed to compounds of formula I wherein there is present at least one (e.g., 1 to 3, or 1-2, or 1) group selected from the group consisting of: —SF₅ and —OSF₅, and when there is more than one group, each group is independently selected.

Thus, one embodiment of this invention is directed to a compound of the formula I:

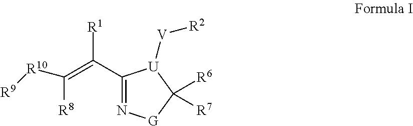

or a pharmaceutically acceptable salt, solvate, or ester thereof, wherein:

U is

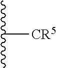

### or N;

G is O or S;

V is selected from the group consisting of a bond, O, —C(O)—, and N(R¹⁴);

R¹ is selected from the group consisting of: H, halo, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 substituents independently selected from the group consisting of the R²¹ groups;

R² is selected from the group consisting of: H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 substituents independently selected from the group consisting of the R²¹ groups; or

R¹ and R² are joined together to form a C5-C8 cycloalkyl or a 5-8 membered heterocyclyl moiety, wherein each of said cycloalkyl or heterocyclyl moiety is optionally substituted with 1-5 substituents independently selected from the group consisting of the R²¹ groups; or

R¹ and R⁸ are taken together to form a bond (i.e., there is a triple bond between the carbon atom to which R¹ was bonded to and the carbon to which R⁸ was bonded to, i.e., the compound of formula I is a compound of formula II:

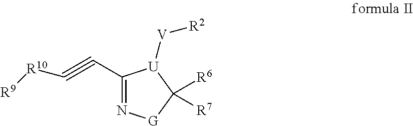

wherein G, U, V, R², R⁶, R⁷, R⁹, and R¹⁰ are as defined for formula I);

R⁵, R⁶ and R⁷ are each independently selected from the group consisting of H, —C(O)R¹⁵, —C(O)OR¹⁵, —C(O)N(R¹⁵)(R¹⁶), —C(═NOR¹⁵)R¹⁶, alkyl-, alkenyl-, alkynyl-, aryl-, arylalkyl-, alkylaryl-, cycloalkyl-, heteroaryl-, heteroarylalkyl-, heterocyclyl- and heterocyclyalkyl-, and wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 substituents independently selected from the group consisting of the R²¹ groups; or

R⁶ and R⁷ are joined together to form a carbocyclic spirocyclic moiety or a heterocyclic spirocyclic moiety wherein each of said carbocyclic spirocyclic moiety and heterocyclic spirocyclic moiety is: (i) optionally substituted with 1-4 substituents independently selected from the group consisting of the the R²¹ groups, or (ii) fused with an aryl, heteroaryl, cycloalkyl or heterocycloalkyl ring, and wherein each of said carbocyclic spirocyclic moiety, heterocyclic spirocyclic moiety, aryl, heteroaryl, cycloalkyl and heterocycloalkyl ring is optionally substituted with 1-4 substituents independently selected from the group consisting of the the R²¹ groups;

R⁸ is selected from the group consisting of H, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, —N₃, —NO₂ and —S(O)₂R²⁴, alkyl-, aryl-, arylalkyl-, alkylaryl-, cycloalkyl-, cycloalkylalkyl-, heteroaryl-, heteroarylalkyl-, heterocyclyl- and heterocyclyalkyl-, and wherein each of said alkyl-, alkenyl-, alkynyl-, aryl-, arylalkyl-, alkylaryl-, cycloalkyl-, heteroaryl-, heteroarylalkyl-, heterocyclyl- and heterocyclyalkyl-moieties is optionally substituted with 1-3 substituents independently selected from the group consisting of the the R²¹ groups;

R¹⁰ is selected from the group consisting of a bond, alkyl-, aryl-, arylalkyl-, arylalkenyl-, alkylaryl-, cycloalkyl-, cycloalkylalkyl-, heteroaryl-, heteroarylalkyl-, heterocyclyl-, heterocyclyalkyl- and the moieties:

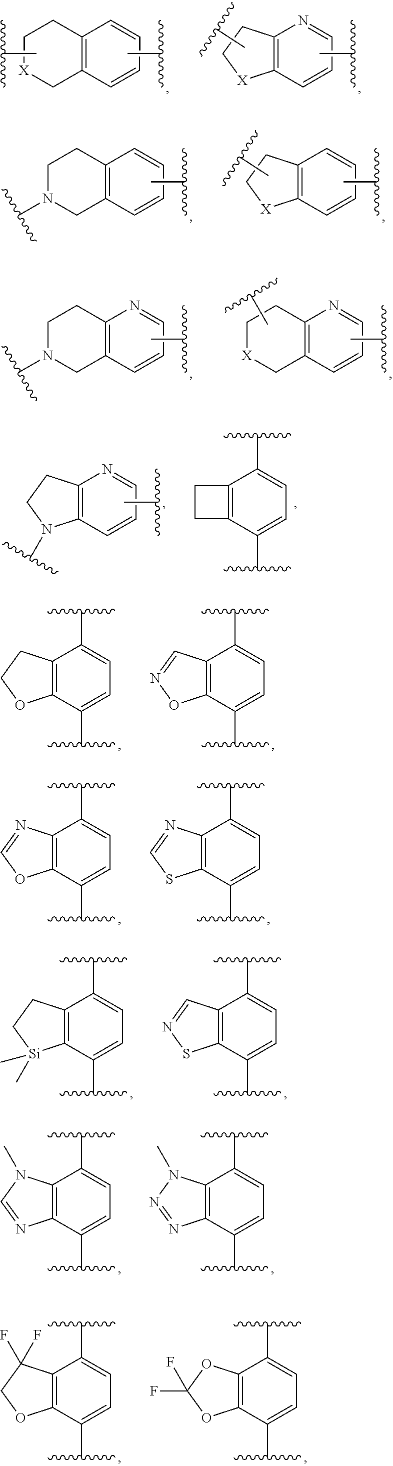

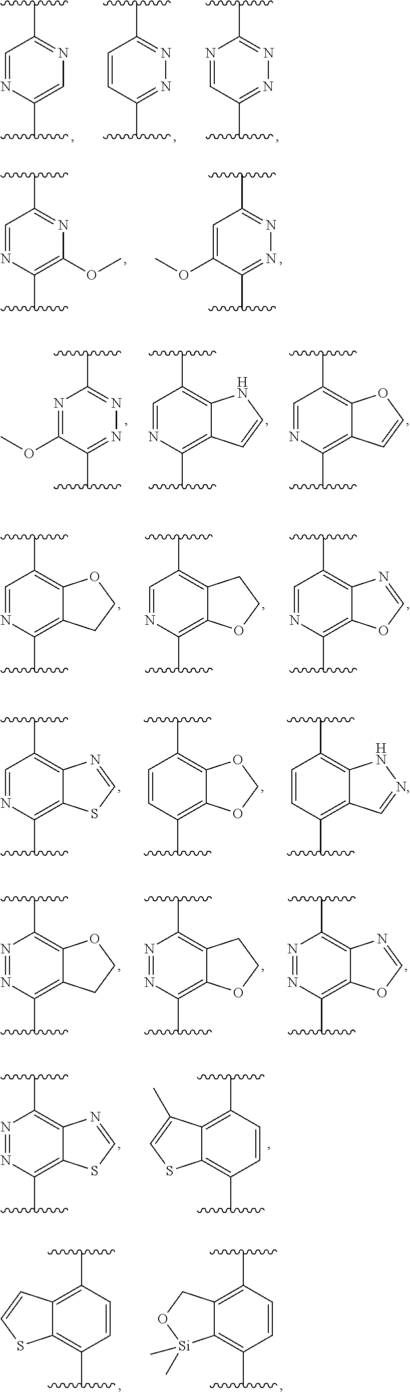

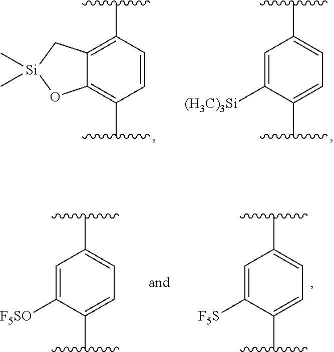

wherein X is selected from the group consisting of: O, N(R¹⁴) or S; and wherein each of said R¹⁰ groups (excluding the bond) is optionally substituted with 1-3 substituents independently selected from the group consisting of the R²¹ groups; or, alternatively, R⁸ and R¹⁰, together with the carbon atom to which they are bound, can form a C₄-C₇ carbocyclic (e.g., cycloalkyl) ring, or a 4-7 membered heterocyclyl ring, or a C₄-C₇ carbocyclenyl (e.g., cycloalkenyl) ring, or a 4-7 membered heterocyclenyl ring; and wherein said carbocyclic ring, heterocyclyl ring, carbocyclenyl ring, or heterocyclenyl ring is optionally substituted with 1-5 independently selected R²¹ substituents;

R⁹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, and wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-3 substituents independently selected from the group consisting of the R²¹ groups;

R¹⁴ is selected from the group consisting of H, alkyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), and wherein each of the alkyl, cycloalkyl, cycloalkylalkyl-, cycloalkenyl, heterocyclyl, heterocyclylalkyl-, aryl, arylalkyl-, heteroaryl, heteroarylalkyl-groups is optionally substituted with 1-5 independently selected R²¹ groups;

R¹⁵, R¹⁶ and R¹⁷ are independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ₙ-alkyl-, (R¹⁸)ₙ-cycloalkyl-, (R¹⁸)ₙ-cycloalkylalkyl-, (R¹⁸)ₙ-heterocyclyl-, (R¹⁸)ₙ-heterocyclylalkyl-, (R¹⁸)ₙ-aryl-, (R¹⁸)ₙ-arylalkyl-, (R¹⁸)ₙ-heteroaryl- and (R¹⁸)ₙ-heteroarylalkyl-;

n is 1 to 5;

Each R¹⁸ is independently selected from the group consisting of: alkyl, alkenyl, alkynyl, aryl, arylalkyl-, arylalkenyl-, arylalkynyl-, —NO₂, halo, heteroaryl, —CF₃, —CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, \ —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl); or

two R¹⁸ moieties on adjacent carbons can be linked together to form:

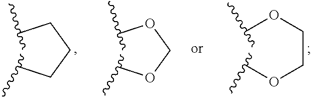

R¹⁹ is selected from the group consisting of: alkyl, cycloalkyl, aryl, arylalkyl and heteroarylalkyl;

R²⁰ is selected from the group consisting of: alkyl, cycloalkyl, aryl, halo substituted aryl, arylalkyl, heteroaryl and heteroarylalkyl;

Each R²¹ is independently selected from the group consisting of: alkyl, alkenyl, alkynyl, cycloalkyl, cycloalkylalkyl-, cycloalkenyl, heterocycloalkyl, heterocycloalkylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SF₅, —OSF₅, —Si(R²⁴)₃ wherein each R²⁴ is independently selected —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, —N₃, —NO₂ and —S(O)₂R²⁴;

wherein each of the alkyl, cycloalkenyl, cycloalkyl, cycloalkylalkyl-, heterocycloalkyl, heterocycloalkylalkyl-, aryl, arylalkyl-, heteroaryl, heteroarylalkyl-, alkenyl and alkynyl R²¹ groups is optionally substituted with 1 to 5 independently selected R²² groups;

Each R²² is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R²⁴)₃ wherein each R²⁴ is independently selected —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, —NO₂, —S(O)R¹⁵ and —S(O)₂R²⁴; and

Each R²⁴ is independently selected from the group consisting of alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ₙ-alkyl-, (R¹⁸)ₙ-cycloalkyl-, (R¹⁸)ₙ-cycloalkylalkyl-, (R¹⁸)ₙ-heterocyclyl-, (R¹⁸)ₙ-heterocyclylalkyl-, (R¹⁸)ₙ-aryl-, (R¹⁸)ₙ-arylalkyl-, (R¹⁸)ₙ-heteroaryl- and (R¹⁸)ₙ-heteroarylalkyl- (wherein R¹⁸ and n are as defined above); and

with the proviso that there is present at least one (e.g., 1 to 3, or 1-2, or 1) group selected from the group consisting of: —SF₅ and —OSF₅, and wherein there is more than one group, each group is independently selected.

Those skilled in the art will appreciate that the —SF₅ and —OSF₅ groups are present in the compounds of formula I: (a) due to the presence of at least one R²¹ group that is selected from the group consisting of: —SF₅ and —OSF₅, or (b) due to the presence of at least one R²² substituent on at least one R²¹ group, wherein said R²² substituent is selected from the group consisting of: —SF₅ and —OSF₅, and wherein said R²¹ group is other than a —SF₅ or —OSF₅ group.

The compounds of this invention are useful for treating central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

Thus, for example, the compounds of this invention can be used to treat the following diseases or conditions: Alzheimers disease, mild cognitive impairment (MCI), Downs Syndrome, Glaucoma (Guo et. al., Proc. Natl. Acad. Sci. USA 104, 13444-13449 (2007)), Cerebral amyloid angiopathy, stroke or dementia (Frangione et al., Amyloid: J. Protein folding Disord. 8, suppl. 1, 36-42 (2001), Microgliosis and brain inflammation (M P Lamber, Proc. Natl. Acad. Sci. USA 95, 6448-53 (1998)), and Olfactory function loss (Getchell, et. al. Neurobiology of Aging, 663-673, 24, 2003).

In one embodiment of this invention one group selected from the group consisting of: —SF₅, —OSF₅, and —Si(R²⁴)₃ (wherein each R²⁴ is independently selected) is present in the compounds of formula I.

In another embodiment of this invention two groups selected from the group consisting of: —SF₅, —OSF₅, and —Si(R²⁴)₃ (wherein each R²⁴ is independently selected) are present in the compounds of formula I.

In another embodiment of this invention three groups selected from the group consisting of: —SF₅, —OSF₅, and —Si(R²⁴)₃ (wherein each R²⁴ is independently selected) are present in the compounds of formula I.

In another embodiment of this invention two groups selected from the group consisting of: —SF₅, —OSF₅, and —Si(R²⁴)₃ (wherein each R²⁴ is independently selected) are present in the compounds of formula I, wherein at least one group is other than —Si(R²⁴)₃.

In another embodiment of this invention three groups selected from the group consisting of: —SF₅, —OSF₅, and —Si(R²⁴)₃ (wherein each R²⁴ is independently selected) are present in the compounds of formula I, wherein at least one group is other than —Si(R²⁴)₃.

In another embodiment of this invention one group selected from the group consisting of: —SF₅, —OSF₅, and —Si(R²⁴)₃ (wherein each R²⁴ is independently selected from the group consisting of alkyl (e.g., methyl and ethyl) and aryl (e.g., phenyl)) is present in the compounds of formula I.

In another embodiment of this invention two groups selected from the group consisting of: —SF₅, —OSF₅, and —Si(R²⁴)₃ (wherein each R²⁴ is independently selected from the group consisting of alkyl (e.g., methyl and ethyl) and aryl (e.g., phenyl)) are present in the compounds of formula I.

In another embodiment of this invention three groups selected from the group consisting of: —SF₅, —OSF₅, and —Si(R²⁴)₃ (wherein each R²⁴ is independently selected from the group consisting of alkyl (e.g., methyl and ethyl) and aryl (e.g., phenyl)) are present in the compounds of formula I.

In another embodiment of this invention two groups selected from the group consisting of: —SF₅, —OSF₅, and —Si(R²⁴)₃ (wherein each R²⁴ is independently selected from the group consisting of alkyl (e.g., methyl and ethyl) and aryl (e.g., phenyl)) are present in the compounds of formula I, wherein at least one group is other than —Si(R²⁴)₃.

In another embodiment of this invention three groups selected from the group consisting of: —SF₅, —OSF₅, and —Si(R²⁴)₃ (wherein each R²⁴ is independently selected from the group consisting of alkyl (e.g., methyl and ethyl) and aryl (e.g., phenyl)) are present in the compounds of formula I, wherein at least one group is other than —Si(R²⁴)₃.

In another embodiment of this invention one group selected from the group consisting of: —SF₅, —OSF₅, and —Si(R²⁴)₃ (wherein each R²⁴ is independently selected from the group consisting of methyl, ethyl and phenyl) is present in the compounds of formula I.

In another embodiment of this invention two groups selected from the group consisting of: —SF₅, —OSF₅, and —Si(R²⁴)₃ (wherein each R²⁴ is independently selected from the group consisting of methyl, ethyl and phenyl) are present in the compounds of formula I.

In another embodiment of this invention three groups selected from the group consisting of: —SF₅, —OSF₅, and —Si(R²⁴)₃ (wherein each R²⁴ is independently selected from the group consisting of methyl, ethyl and phenyl) are present in the compounds of formula I.

In another embodiment of this invention two groups selected from the group consisting of: —SF₅, —OSF₅, and —Si(R²⁴)₃ (wherein each R²⁴ is independently selected from the group consisting of methyl, ethyl and phenyl) are present in the compounds of formula I, wherein at least one group is other than —Si(R²⁴)₃.

In another embodiment of this invention three groups selected from the group consisting of: —SF₅, —OSF₅, and —Si(R²⁴)₃ (wherein each R²⁴ is independently selected from the group consisting of methyl, ethyl and phenyl) are present in the compounds of formula I, wherein at least one group is other than —Si(R²⁴)₃.

In another embodiment of this invention one group selected from the group consisting of: —SF₅, —OSF₅, and —Si(R²⁴)₃ (wherein each R²⁴ is independently selected from the group consisting of methyl and ethyl) is present in the compounds of formula I.

In another embodiment of this invention two groups selected from the group consisting of: —SF₅, —OSF₅, and —Si(R²⁴)₃ (wherein each R²⁴ is independently selected from the group consisting of methyl and ethyl) are present in the compounds of formula I.

In another embodiment of this invention three groups selected from the group consisting of: —SF₅, —OSF₅, and —Si(R²⁴)₃ (wherein each R²⁴ is independently selected from the group consisting of methyl and ethyl) are present in the compounds of formula I.

In another embodiment of this invention two groups selected from the group consisting of: —SF₅, —OSF₅, and —Si(R²⁴)₃ (wherein each R²⁴ is independently selected from the group consisting of methyl and ethyl) are present in the compounds of formula I, wherein at least one group is other than —Si(R²⁴)₃.

In another embodiment of this invention three groups selected from the group consisting of: —SF₅, —OSF₅, and —Si(R²⁴)₃ (wherein each R²⁴ is independently selected from the group consisting of methyl and ethyl) are present in the compounds of formula I, wherein at least one group is other than —Si(R²⁴)₃.

In another embodiment of this invention one group selected from the group consisting of: —SF₅, —OSF₅, and —Si(R²⁴)₃ is present in the compounds of formula I, and said —Si(R²⁴)₃ group is selected from the group consisting of: —Si(CH₃)₃, —Si(CH₃)₂phenyl, and —Si(CH₂CH₃)₂CH₃.

In another embodiment of this invention two groups selected from the group consisting of: —SF₅, —OSF₅, and —Si(R²⁴)₃ are present in the compounds of formula I, and said —Si(R²⁴)₃ group is selected from the group consisting of: —Si(CH₃)₃, —Si(CH₃)₂phenyl, and —Si(CH₂CH₃)₂CH₃.

In another embodiment of this invention three groups selected from the group consisting of: —SF₅, —OSF₅, and —Si(R²⁴)₃ are present in the compounds of formula I, and said —Si(R²⁴)₃ group is selected from the group consisting of: —Si(CH₃)₃, —Si(CH₃)₂phenyl, and —Si(CH₂CH₃)₂CH₃.

In another embodiment of this invention two groups selected from the group consisting of: —SF₅, —OSF₅, and —Si(R²⁴)₃ are present in the compounds of formula I, wherein at least one group is other than —Si(R²⁴)₃, and said —Si(R²⁴)₃ group is selected from the group consisting of: —Si(CH₃)₃, —Si(CH₃)₂phenyl, and —Si(CH₂CH₃)₂CH₃.

In another embodiment of this invention three groups selected from the group consisting of: —SF₅, —OSF₅, and —Si(R²⁴)₃ are present in the compounds of formula I, wherein at least one group is other than —Si(R²⁴)₃, and said —Si(R²⁴)₃ group is selected from the group consisting of: —Si(CH₃)₃, —Si(CH₃)₂phenyl, and —Si(CH₂CH₃)₂CH₃.

In another embodiment of this invention one group selected from the group consisting of: —SF₅, —OSF₅, and —Si(R²⁴)₃ is present in the compounds of formula I, and said —Si(R²⁴)₃ group is selected from the group consisting of: —Si(CH₃)₃ and —Si(CH₂CH₃)₂CH₃.

In another embodiment of this invention two groups selected from the group consisting of: —SF₅, —OSF₅, and —Si(R²⁴)₃ are present in the compounds of formula I, and said —Si(R²⁴)₃ group is selected from the group consisting of: —Si(CH₃)₃ and —Si(CH₂CH₃)₂CH₃.

In another embodiment of this invention three groups selected from the group consisting of: —SF₅, —OSF₅, and —Si(R²⁴)₃ are present in the compounds of formula I, and said —Si(R²⁴)₃ group is selected from the group consisting of: —Si(CH₃)₃ and —Si(CH₂CH₃)₂CH₃.

In another embodiment of this invention two groups selected from the group consisting of: —SF₅, —OSF₅, and —Si(R²⁴)₃ are present in the compounds of formula I, wherein at least one group is other than —Si(R²⁴)₃, and said —Si(R²⁴)₃ group is selected from the group consisting of: —Si(CH₃)₃ and —Si(CH₂CH₃)₂CH₃.

In another embodiment of this invention three groups selected from the group consisting of: —SF₅, —OSF₅, and —Si(R²⁴)₃ are present in the compounds of formula I, wherein at least one group is other than —Si(R²⁴)₃, and said —Si(R²⁴)₃ group is selected from the group consisting of: —Si(CH₃)₃, —Si(CH₃)₂phenyl, and —Si(CH₂CH₃)₂CH₃.

In another embodiment of this invention one group selected from the group consisting of: —SF₅, —OSF₅, and —Si(CH₃)₃.

In another embodiment of this invention two groups selected from the group consisting of: —SF₅, —OSF₅, and —Si(CH₃)₃ are present in the compounds of formula I.

In another embodiment of this invention three groups selected from the group consisting of: —SF₅, —OSF₅, and —Si(CH₃)₃ are present in the compounds of formula I.

In another embodiment of this invention two groups selected from the group consisting of: —SF₅, —OSF₅, and —Si(CH₃)₃ are present in the compounds of formula I, wherein at least one group is other than —Si(CH₃)₃.

In another embodiment of this invention three groups selected from the group consisting of: —SF₅, —OSF₅, and —Si(R²⁴)₃ are present in the compounds of formula I, wherein at least one group is other than —Si(CH₃)₃.

In another embodiment of this invention one group selected from the group consisting of: —SF₅ and —OSF₅ is present in the compounds of formula I.

In another embodiment of this invention two groups selected from the group consisting of: —SF₅ and —OSF₅ are present in the compounds of formula I.

In another embodiment of this invention three groups selected from the group consisting of: —SF₅ and —OSF₅ are present in the compounds of formula I.

In another embodiment of this invention one —SF₅ group is present in the compounds of formula I.

In another embodiment of this invention two —SF₅ groups are present in the compounds of formula I.

In another embodiment of this invention three —SF₅ groups are present in the compounds of formula I.

In another embodiment of this invention one —OSF₅ group is present in the compounds of formula I.

In another embodiment of this invention two —OSF₅ groups are present in the compounds of formula I.

In another embodiment of this invention three —OSF₅ groups are present in the compounds of formula I.

In another embodiment of this invention one group selected from the group consisting of: —SF₅ and —OSF₅ is present in the compounds of formula I, no —Si(R²⁴)₃ groups are present, and R¹⁰ is any of the groups defined in formula I.

In another embodiment of this invention two groups selected from the group consisting of: —SF₅ and —OSF₅ are present in the compounds of formula I, no —Si(R²⁴)₃ groups are present, and R¹⁰ is any of the groups defined in formula I.

In another embodiment of this invention three groups selected from the group consisting of: —SF₅ and —OSF₅ are present in the compounds of formula I, no —Si(R²⁴)₃ groups are present, and R¹⁰ is any of the groups defined in formula I.

In another embodiment of this invention one —SF₅ group is present in the compounds of formula I, no —Si(R²⁴)₃ groups are present, and R¹⁰ is any of the groups defined in formula I.

In another embodiment of this invention two —SF₅ groups are present in the compounds of formula I, no —Si(R²⁴)₃ groups are present, and R¹⁰ is any of the groups defined in formula I.

In another embodiment of this invention three —SF₅ groups are present in the compounds of formula I, no —Si(R²⁴)₃ groups are present, and R¹⁰ is any of the groups defined in formula I.

In another embodiment of this invention one —OSF₅ group is present in the compounds of formula I, no —Si(R²⁴)₃ groups are present, and R¹⁰ is any of the groups defined in formula I.

In another embodiment of this invention two —OSF₅ groups are present in the compounds of formula I, no —Si(R²⁴)₃ groups are present, and R¹⁰ is any of the groups defined in formula I.

In another embodiment of this invention three —OSF₅ groups are present in the compounds of formula I, no —Si(R²⁴)₃ groups are present, and R¹⁰ is any of the groups defined in formula I.

In another embodiment of this invention one —Si(R²⁴)₃ (wherein each R²⁴ is independently selected) group is present in the compounds of formula I.

In another embodiment of this invention two —Si(R²⁴)₃ (wherein each R²⁴ is independently selected) groups are present in the compounds of formula I.

In another embodiment of this invention three —Si(R²⁴)₃ (wherein each R²⁴ is independently selected) groups are present in the compounds of formula I.

In another embodiment of this invention one —Si(R²⁴)₃ (wherein each R²⁴ is independently selected from the group consisting of alkyl (e.g., methyl and ethyl) and aryl (e.g., phenyl)) is present in the compounds of formula I.

In another embodiment of this invention two —Si(R²⁴)₃ (wherein each R²⁴ is independently selected from the group consisting of alkyl (e.g., methyl and ethyl) and aryl (e.g., phenyl)) is present in the compounds of formula I.

In another embodiment of this invention three —Si(R²⁴)₃ (wherein each R²⁴ is independently selected from the group consisting of alkyl (e.g., methyl and ethyl) and aryl (e.g., phenyl)) is present in the compounds of formula I.

In another embodiment of this invention one —Si(R²⁴)₃ (wherein each R²⁴ is independently selected from the group consisting of methyl, ethyl and phenyl) is present in the compounds of formula I.

In another embodiment of this invention two —Si(R²⁴)₃ (wherein each R²⁴ is independently selected from the group consisting of methyl, ethyl and phenyl) is present in the compounds of formula I.

In another embodiment of this invention three —Si(R²⁴)₃ (wherein each R²⁴ is independently selected from the group consisting of methyl, ethyl and phenyl) is present in the compounds of formula I.

In another embodiment of this invention one —Si(R²⁴)₃ (wherein each R²⁴ is independently selected from the group consisting of methyl and ethyl) is present in the compounds of formula I.

In another embodiment of this invention two —Si(R²⁴)₃ (wherein each R²⁴ is independently selected from the group consisting of methyl and ethyl) is present in the compounds of formula I.

In another embodiment of this invention three —Si(R²⁴)₃ (wherein each R²⁴ is independently selected from the group consisting of methyl and ethyl) is present in the compounds of formula I.

In another embodiment of this invention one —Si(R²⁴)₃ group is present in the compounds of formula I, and said —Si(R²⁴)₃ group is selected from the group consisting of: —Si(CH₃)₃, —Si(CH₃)₂phenyl, and —Si(CH₂CH₃)₂CH₃.

In another embodiment of this invention two —Si(R²⁴)₃ groups are present in the compounds of formula I, and said —Si(R²⁴)₃ groups are independently selected from the group consisting of: —Si(CH₃)₃, —Si(CH₃)₂phenyl, and —Si(CH₂CH₃)₂CH₃.

In another embodiment of this invention three —Si(R²⁴)₃ groups are present in the compounds of formula I, and said —Si(R²⁴)₃ groups are independently selected from the group consisting of: —Si(CH₃)₃, —Si(CH₃)₂phenyl, and —Si(CH₂CH₃)₂CH₃.

In another embodiment of this invention one —Si(R²⁴)₃ group is present in the compounds of formula I, and said —Si(R²⁴)₃ group is selected from the group consisting of: —Si(CH₃)₃ and —Si(CH₂CH₃)₂CH₃.

In another embodiment of this invention two —Si(R²⁴)₃ groups are present in the compounds of formula I, and said —Si(R²⁴)₃ groups are independently selected from the group consisting of: —Si(CH₃)₃ and —Si(CH₂CH₃)₂CH₃.

In another embodiment of this invention three —Si(R²⁴)₃ groups are present in the compounds of formula I, and said —Si(R²⁴)₃ groups are independently selected from the group consisting of: —Si(CH₃)₃ and —Si(CH₂CH₃)₂CH₃.

In another embodiment of this invention one —Si(R²⁴)₃ group is present in the compounds of formula I, and said —Si(R²⁴)₃ group is —Si(CH₃)₃.

In another embodiment of this invention two —Si(R²⁴)₃ groups are present in the compounds of formula I, and said —Si(R²⁴)₃ groups are —Si(CH₃)₃.

In another embodiment of this invention three —Si(R²⁴)₃ groups are present in the compounds of formula I, and said —Si(R²⁴)₃ groups are —Si(CH₃)₃.

In another embodiment of this invention one group selected from the group consisting of: —SF₅, —OSF₅, —Si(CH₃)₃, —Si(CH₃)₂phenyl, and —Si(CH₂CH₃)₂CH₃) is present in the compounds of formula I.

In another embodiment of this invention one group selected from the group consisting of: —SF₅, —OSF₅, —Si(CH₃)₃, and —Si(CH₂CH₃)₂CH₃) is present in the compounds of formula I.

In another embodiment of this invention one group selected from the group consisting of: —SF₅, —OSF₅, and —Si(CH₃)₃, is present in the compounds of formula I.

In another embodiment of this invention one —SF₅ group is present in the compounds of formula I, and one or two additional groups selected from the group consisting of: —SF₅, —OSF₅, and —Si(R²⁴)₃ (wherein each R²⁴ is independently selected) are also present in the compounds of formula I.

In another embodiment of this invention one —SF₅ group is present in the compounds of formula I, and one or two additional groups selected from the group consisting of: —OSF₅, and —Si(R²⁴)₃ (wherein each R²⁴ is independently selected) are also present in the compounds of formula I.

In another embodiment of this invention one —OSF₅ group is present in the compounds of formula I, and one or two additional groups selected from the group consisting of: —SF₅, —OSF₅, and —Si(R²⁴)₃ (wherein each R²⁴ is independently selected) are also present in the compounds of formula I.

In another embodiment of this invention one —OSF₅ group is present in the compounds of formula I, and one or two additional groups selected from the group consisting of: —SF₅ and —Si(R²⁴)₃ (wherein each R²⁴ is independently selected) are also present in the compounds of formula I.

In another embodiment of this invention one —SF₅ group is present in the compounds of formula I, and one or two additional groups selected from the group consisting of: —SF₅ and —OSF₅ are also present in the compounds of formula I.

In another embodiment of this invention one —OSF₅ group is present in the compounds of formula I, and one or two additional groups selected from the group consisting of: —SF₅ and —OSF₅ are also present in the compounds of formula I.

In another embodiment of this invention one —Si(R²⁴)₃ (wherein each R²⁴ is independently selected) group is present in the compounds of formula I, and one or two groups selected from the group consisting of: —SF₅, —OSF₅, and —Si(R²⁴)₃ (wherein each R²⁴ is independently selected) are also present in the compounds of formula I.

In another embodiment of this invention one —Si(R²⁴)₃ (wherein each R²⁴ is independently selected) group is present in the compounds of formula I, and one or two groups selected from the group consisting of: —SF₅ and —OSF₅ are also present in the compounds of formula I.

In another embodiment of this invention at least one group selected from the group consisting of: —SF₅, —OSF₅, and —Si(R²⁴)₃ (wherein each R²⁴ is independently selected from the group consisting of alkyl (e.g., methyl and ethyl) and aryl (e.g., phenyl)) is present in the compounds of formula I.

In another embodiment of this invention at least one group selected from the group consisting of: —SF₅, —OSF₅, and —Si(R²⁴)₃ (wherein each R¹⁵ is independently selected from the group consisting of alkyl (e.g., methyl and ethyl) and phenyl) is present in the compounds of formula I.

In another embodiment of this invention at least one group selected from the group consisting of: —SF₅, —OSF₅, and —Si(R¹⁵)₃ (wherein each R¹⁵ is independently selected from the group consisting of methyl, ethyl and phenyl) is present in the compounds of formula I.

In another embodiment of this invention at least one group selected from the group consisting of: —SF₅, —OSF₅, —Si(CH₃)₃, —Si(CH₃)₂phenyl, and —Si(CH₂CH₃)₂CH₃) is present in the compounds of formula I.

In another embodiment of this invention at least one group selected from the group consisting of: —SF₅, —OSF₅, and —Si(CH₃)₃ is present in the compounds of formula I.

In another embodiment of this invention one group selected from the group consisting of: —SF₅, —OSF₅, and —Si(R¹⁵)₃ (wherein each R¹⁵ is independently selected) is present in the compounds of formula I.

In another embodiment of this invention one group selected from the group consisting of: —SF₅, —OSF₅, and —Si(R¹⁵)₃ (wherein each R¹⁵ is independently selected from the group consisting of alkyl (e.g., methyl and ethyl) and aryl (e.g., phenyl)) is present in the compounds of formula I.

In another embodiment of this invention one group selected from the group consisting of: —SF₅, —OSF₅, and —Si(R¹⁵)₃ (wherein each R¹⁵ is independently selected from the group consisting of alkyl (e.g., methyl and ethyl) and phenyl) is present in the compounds of formula I.

In another embodiment of this invention one group selected from the group consisting of: —SF₅, —OSF₅, and —Si(R¹⁵)₃ (wherein each R¹⁵ is independently selected from the group consisting of methyl, ethyl and phenyl) is present in the compounds of formula I.

In another embodiment of this invention one group selected from the group consisting of: —SF₅, —OSF₅, —Si(CH₃)₃, —Si(CH₃)₂phenyl, and —Si(CH₂CH₃)₂CH₃) is present in the compounds of formula I.

In another embodiment of this invention one group selected from the group consisting of: —SF₅, —OSF₅, —Si(CH₃)₃, and —Si(CH₂CH₃)₂CH₃) is present in the compounds of formula I.

In another embodiment of this invention one group selected from the group consisting of: —SF₅, —OSF₅, and —Si(CH₃)₃, is present in the compounds of formula I.

In another embodiment of this invention two groups selected from the group consisting of: —SF₅, —OSF₅, and —Si(R¹⁵)₃ (wherein each R¹⁵ is independently selected) are present in the compounds of formula I.

In another embodiment of this invention two groups independently selected from the group consisting of: —SF₅, —OSF₅, and —Si(R¹⁵)₃ (wherein each R¹⁵ is independently selected from the group consisting of alkyl (e.g., methyl and ethyl) and aryl (e.g., phenyl)) are present in the compounds of formula I.

In another embodiment of this invention two groups independently selected from the group consisting of: —SF₅, —OSF₅, and —Si(R¹⁵)₃ (wherein each R¹⁵ is independently selected from the group consisting of alkyl (e.g., methyl and ethyl) and phenyl) are present in the compounds of formula I.

In another embodiment of this invention two groups selected from the group consisting of: —SF₅, —OSF₅, and —Si(R¹⁵)₃ (wherein each R¹⁵ is independently selected from the group consisting of methyl, ethyl and phenyl) are present in the compounds of formula I.

In another embodiment of this invention two groups independently selected from the group consisting of: —SF₅, —OSF₅, —Si(CH₃)₃, —Si(CH₃)₂phenyl, and —Si(CH₂CH₃)₂CH₃) is present in the compounds of formula I.

In another embodiment of this invention two groups independently selected from the group consisting of: —SF₅, —OSF₅, —Si(CH₃)₃, and —Si(CH₂CH₃)₂CH₃) are present in the compounds of formula I.

In another embodiment of this invention two groups independently selected from the group consisting of: —SF₅, —OSF₅, and —Si(CH₃)₃ are present in the compounds of formula I.

In another embodiment of this invention three groups selected from the group consisting of: —SF₅, —OSF₅, and —Si(R¹⁵)₃ (wherein each R¹⁵ is independently selected) are present in the compounds of formula I.

In another embodiment of this invention three groups independently selected from the group consisting of: —SF₅, —OSF₅, and —Si(R¹⁵)₃ (wherein each R¹⁵ is independently selected from the group consisting of alkyl (e.g., methyl and ethyl) and aryl (e.g., phenyl)) are present in the compounds of formula I.

In another embodiment of this invention three groups independently selected from the group consisting of: —SF₅, —OSF₅, and —Si(R¹⁵)₃ (wherein each R¹⁵ is independently selected from the group consisting of alkyl (e.g., methyl and ethyl) and phenyl) are present in the compounds of formula I.

In another embodiment of this invention three groups selected from the group consisting of: —SF₅, —OSF₅, and —Si(R¹⁵)₃ (wherein each R¹⁵ is independently selected from the group consisting of methyl, ethyl and phenyl) are present in the compounds of formula I.

In another embodiment of this invention three groups independently selected from the group consisting of: —SF₅, —OSF₅, —Si(CH₃)₃, —Si(CH₃)₂phenyl, and —Si(CH₂CH₃)₂CH₃) is present in the compounds of formula I.

In another embodiment of this invention three groups independently selected from the group consisting of: —SF₅, —OSF₅, —Si(CH₃)₃, and —Si(CH₂CH₃)₂CH₃) are present in the compounds of formula I.

In another embodiment of this invention three groups independently selected from the group consisting of: —SF₅, —OSF₅, and —Si(CH₃)₃ are present in the compounds of formula I.

In another embodiment of this invention at least one group selected from the group consisting of: —SF₅, —OSF₅, and —Si(R¹⁵)₃ (wherein each R¹⁵ is the same or different alkyl group) is present in the compounds of formula I.

In another embodiment of this invention at least one group selected from the group consisting of: —SF₅, —OSF₅, and —Si(R¹⁵)₃ (wherein each R¹⁵ is independently selected from the group consisting of methyl and ethyl) is present in the compounds of formula I.

In another embodiment of this invention one —SF₅ group is present in the compounds of formula I, and one or two groups selected from the group consisting of: —SF₅ and —OSF₅ are also present in the compounds of formula I.

In another embodiment of this invention one —OSF₅ group is present in the compounds of formula I, and one or two groups selected from the group consisting of: —SF₅ and —OSF₅ are also present in the compounds of formula I.

In another embodiment of this invention R¹⁰ in formula I is selected from the group consisting of:

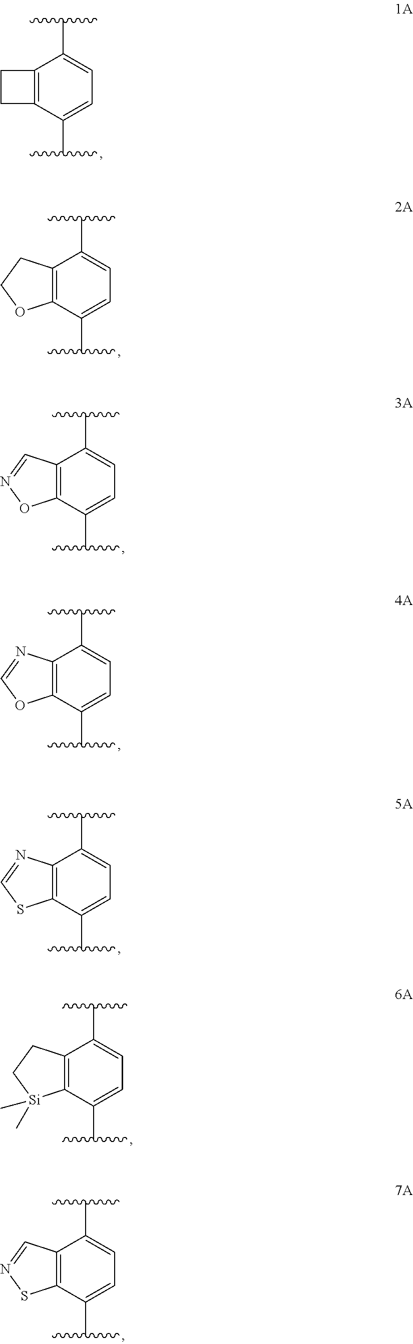

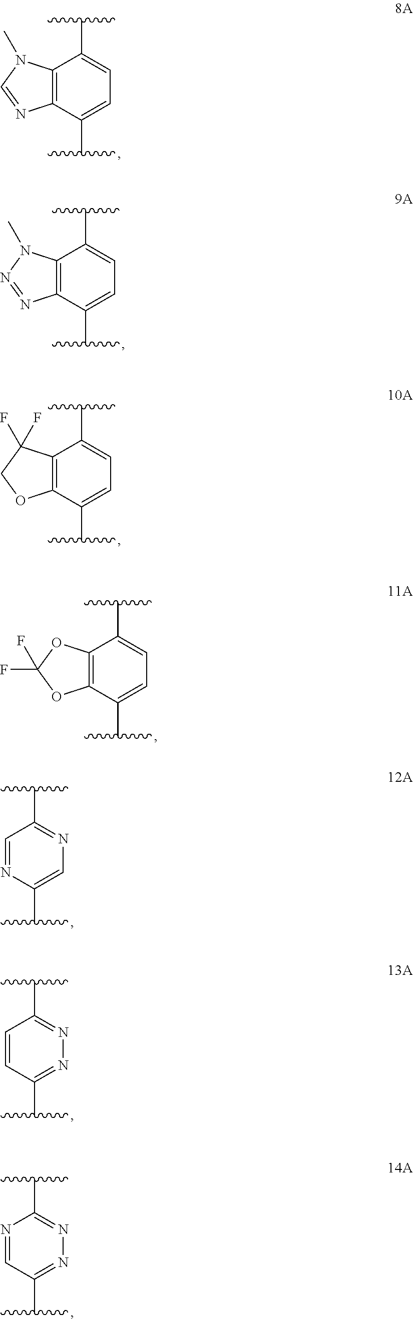

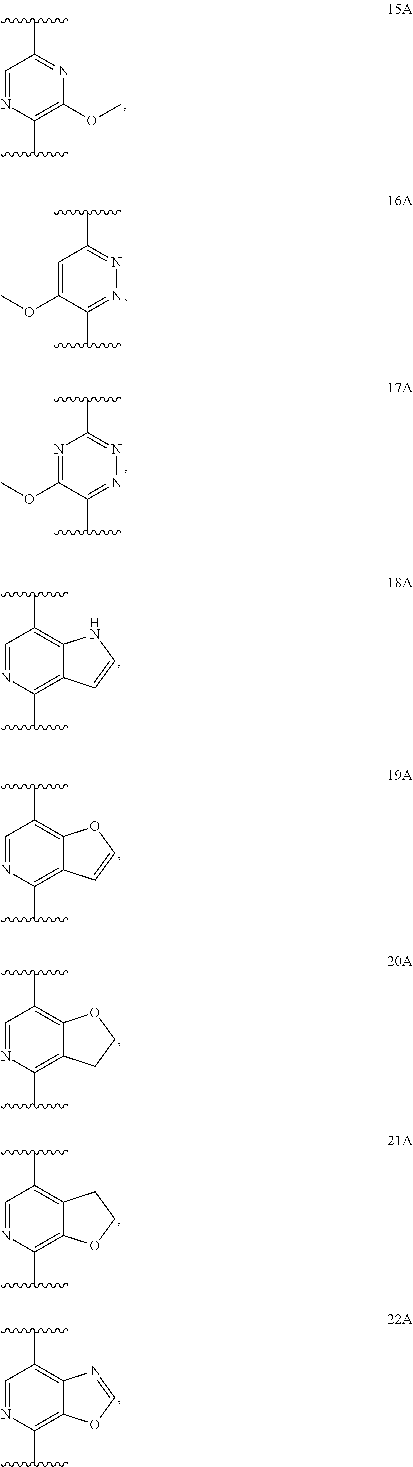

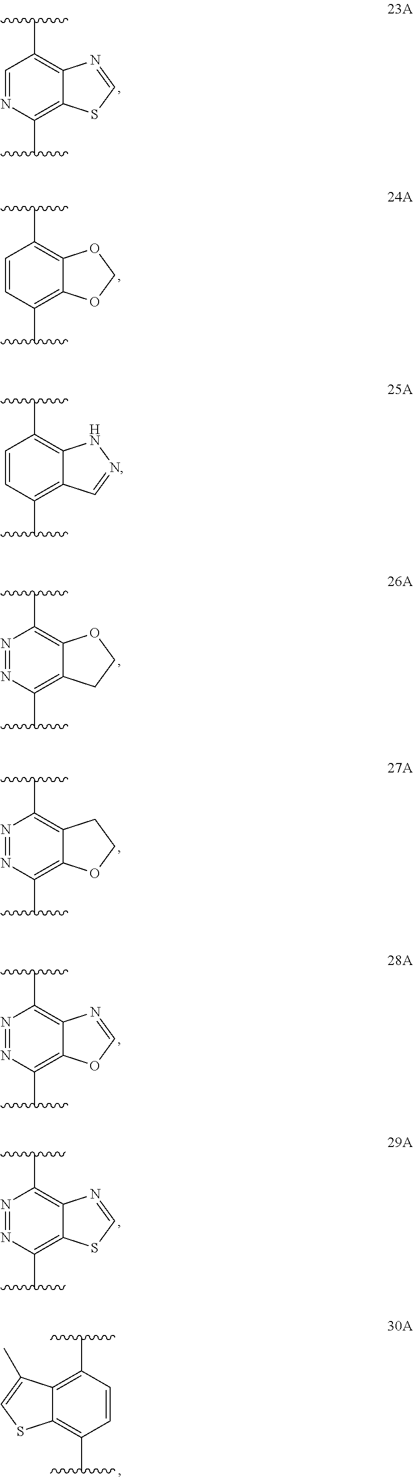

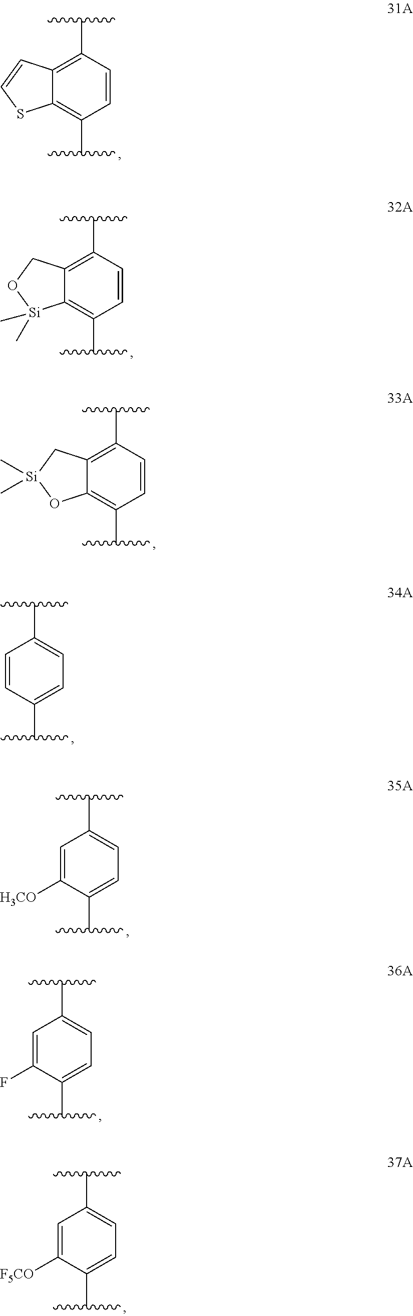

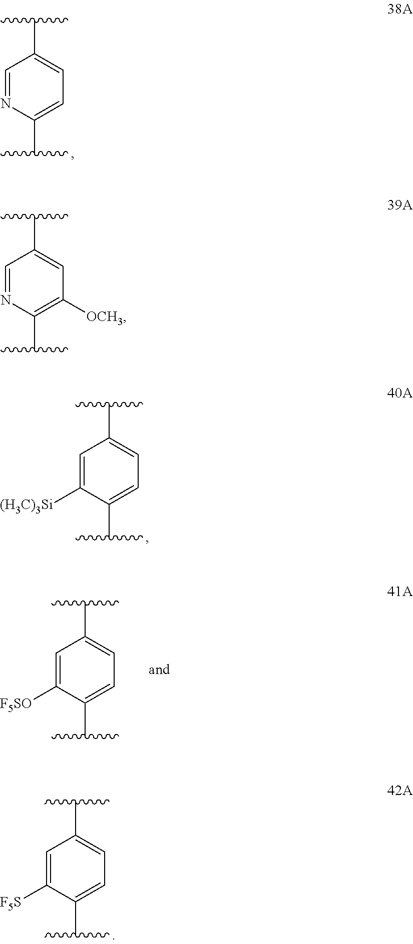

In another embodiment of this invention R¹⁰ is group 1A. In another embodiment of this invention R¹⁰ is group 2A. In another embodiment of this invention R¹⁰ is group 3A. In another embodiment of this invention R¹⁰ is group 4A. In another embodiment of this invention R¹⁰ is group 5A. In another embodiment of this invention R¹⁰ is group 6A. In another embodiment of this invention R¹⁰ is group 7A. In another embodiment of this invention R¹⁰ is group 8A. In another embodiment of this invention R¹⁰ is group 9A. In another embodiment of this invention R¹⁰ is group 10A. In another embodiment of this invention R¹⁰ is group 11A. In another embodiment of this invention R¹⁰ is group 12A. In another embodiment of this invention R¹⁰ is group 13A. In another embodiment of this invention R¹⁰ is group 14A. In another embodiment of this invention R¹⁰ is group 15A. In another embodiment of this invention R¹⁰ is group 16A. In another embodiment of this invention R¹⁰ is group 17A. In another embodiment of this invention R¹⁰ is group 18A. In another embodiment of this invention R¹⁰ is group 19A. In another embodiment of this invention R¹⁰ is group 20A. In another embodiment of this invention R¹⁰ is group 21A. In another embodiment of this invention R¹⁰ is group 22A. In another embodiment of this invention R¹⁰ is group 23A. In another embodiment of this invention R¹⁰ is group 24A. In another embodiment of this invention R¹⁰ is group 25A. In another embodiment of this invention R¹⁰ is group 26A. In another embodiment of this invention R¹⁰ is group 27A. In another embodiment of this invention R¹⁰ is group 28A. In another embodiment of this invention R¹⁰ is group 29A. In another embodiment of this invention R¹⁰ is group 30A. In another embodiment of this invention R¹⁰ is group 31A. In another embodiment of this invention R¹⁰ is group 32A. In another embodiment of this invention R¹⁰ is group 33A. In another embodiment of this invention R¹⁰ is group 34A. In another embodiment of this invention R¹⁰ is group 35A. In another embodiment of this invention R¹⁰ is group 36A. In another embodiment of this invention R¹⁰ is group 37A. In another embodiment of this invention R¹⁰ is group 38A. In another embodiment of this invention R¹⁰ is group 39A. In another embodiment of this invention R¹⁰ is group 40A. In another embodiment of this invention R¹⁰ is group 41A. In another embodiment of this invention R¹⁰ is group 42A.

In another embodiment of this invention at least one group selected from the group consisting of: —SF₅, —OSF₅, and —Si(R²⁴)₃ (wherein each R²⁴ is independently selected) is present in the compounds of formula I, and R¹⁰ is selected from the group consisting of 1A to 42A.

In another embodiment of this invention one group selected from the group consisting of: —SF₅, —OSF₅, and —Si(R²⁴)₃ (wherein each R²⁴ is independently selected) is present in the compounds of formula I, and R¹⁰ is selected from the group consisting of 1A to 42A.

In another embodiment of this invention two groups selected from the group consisting of: —SF₅, —OSF₅, and —Si(R²⁴)₃ (wherein each R²⁴ is independently selected) are present in the compounds of formula I, and R¹⁰ is selected from the group consisting of 1A to 42A.

In another embodiment of this invention three groups selected from the group consisting of: —SF₅, —OSF₅, and —Si(R²⁴)₃ (wherein each R¹⁵ is independently selected) are present in the compounds of formula I, and R¹⁰ is selected from the group consisting of 1A to 42A.

In another embodiment of this invention at least one group selected from the group consisting of: —SF₅ and —OSF₅ is present in the compounds of formula I, and R¹⁰ is selected from the group consisting of 1A to 42A.

In another embodiment of this invention one group selected from the group consisting of: —SF₅ and —OSF₅ is present in the compounds of formula I, and R¹⁰ is selected from the group consisting of 1A to 42A.

In another embodiment of this invention two groups selected from the group consisting of: —SF₅ and —OSF₅ are present in the compounds of formula I, and R¹⁰ is selected from the group consisting of 1A to 42A.

In another embodiment of this invention three groups selected from the group consisting of: —SF₅ and —OSF₅ are present in the compounds of formula I, and R¹⁰ is selected from the group consisting of 1A to 42A.

In another embodiment of this invention one —SF₅ group is present in the compounds of formula I, and R¹⁰ is selected from the group consisting of 1A to 42A.

In another embodiment of this invention two —SF₅ groups are present in the compounds of formula I, and R¹⁰ is selected from the group consisting of 1A to 42A.

In another embodiment of this invention three —SF₅ groups are present in the compounds of formula I, and R¹⁰ is selected from the group consisting of 1A to 42A.

In another embodiment of this invention one —OSF₅ group is present in the compounds of formula I, and R¹⁰ is selected from the group consisting of 1A to 42A.

In another embodiment of this invention two —OSF₅ groups are present in the compounds of formula I, and R¹⁰ is selected from the group consisting of 1A to 42A.

In another embodiment of this invention three —OSF₅ groups are present in the compounds of formula I, and R¹⁰ is selected from the group consisting of 1A to 42A.

In another embodiment of this invention one —Si(R²⁴)₃ (wherein each R²⁴ is independently selected) group is present in the compounds of formula I, and R¹⁰ is selected from the group consisting of 1A to 42A.

In another embodiment of this invention two —Si(R²⁴)₃ (wherein each R²⁴ is independently selected) groups are present in the compounds of formula I, and R¹⁰ is selected from the group consisting of 1A to 42A.

In another embodiment of this invention three —Si(R²⁴)₃ (wherein each R²⁴ is independently selected) groups are present in the compounds of formula I, and R¹⁰ is selected from the group consisting of 1A to 42A.

In another embodiment of this invention one —Si(R²⁴)₃ (wherein each R²⁴ is independently selected from the group consisting of alkyl (e.g., methyl and ethyl) and aryl (e.g., phenyl)) is present in the compounds of formula I, and R¹⁰ is selected from the group consisting of 1A to 42A.

In another embodiment of this invention two —Si(R²⁴)₃ (wherein each R²⁴ is independently selected from the group consisting of alkyl (e.g., methyl and ethyl) and aryl (e.g., phenyl)) is present in the compounds of formula I, and R¹⁰ is selected from the group consisting of 1A to 42A.

In another embodiment of this invention three —Si(R²⁴)₃ (wherein each R²⁴ is independently selected from the group consisting of alkyl (e.g., methyl and ethyl) and aryl (e.g., phenyl)) is present in the compounds of formula I, and R¹⁰ is selected from the group consisting of 1A to 42A.

In another embodiment of this invention one —Si(R²⁴)₃ (wherein each R²⁴ is independently selected from the group consisting of methyl, ethyl and phenyl) is present in the compounds of formula I, and R¹⁰ is selected from the group consisting of 1A to 42A.

In another embodiment of this invention two —Si(R²⁴)₃ (wherein each R²⁴ is independently selected from the group consisting of methyl, ethyl and phenyl) is present in the compounds of formula I, and R¹⁰ is selected from the group consisting of 1A to 42A.

In another embodiment of this invention three —Si(R²⁴)₃ (wherein each R²⁴ is independently selected from the group consisting of methyl, ethyl and phenyl) is present in the compounds of formula I, and R¹⁰ is selected from the group consisting of 1A to 42A.

In another embodiment of this invention one —Si(R²⁴)₃ (wherein each R²⁴ is independently selected from the group consisting of methyl and ethyl) is present in the compounds of formula I, and R¹⁰ is selected from the group consisting of 1A to 42A.

In another embodiment of this invention two —Si(R²⁴)₃ (wherein each R²⁴ is independently selected from the group consisting of methyl and ethyl) is present in the compounds of formula I, and R¹⁰ is selected from the group consisting of 1A to 42A.

In another embodiment of this invention three —Si(R²⁴)₃ (wherein each R²⁴ is independently selected from the group consisting of methyl and ethyl) is present in the compounds of formula I, and R¹⁰ is selected from the group consisting of 1A to 42A.

In another embodiment of this invention one —Si(R²⁴)₃ group is present in the compounds of formula I, and said —Si(R²⁴)₃ group is selected from the group consisting of: —Si(CH₃)₃, —Si(CH₃)₂phenyl, and —Si(CH₂CH₃)₂CH₃ and R¹⁰ is selected from the group consisting of 1A to 42A.

In another embodiment of this invention two —Si(R²⁴)₃ groups are present in the compounds of formula I, and said —Si(R²⁴)₃ groups are independently selected from the group consisting of: —Si(CH₃)₃, —Si(CH₃)₂phenyl, and —Si(CH₂CH₃)₂CH₃ and R¹⁰ is selected from the group consisting of 1A to 42A.

In another embodiment of this invention three —Si(R²⁴)₃ groups are present in the compounds of formula I, and said —Si(R²⁴)₃ groups are independently selected from the group consisting of: —Si(CH₃)₃, —Si(CH₃)₂phenyl, and —Si(CH₂CH₃)₂CH₃ and R¹⁰ is selected from the group consisting of 1A to 42A.

In another embodiment of this invention one —Si(R²⁴)₃ group is present in the compounds of formula I, and said —Si(R²⁴)₃ group is selected from the group consisting of: —Si(CH₃)₃ and —Si(CH₂CH₃)₂CH₃ and R¹⁰ is selected from the group consisting of 1A to 42A.

In another embodiment of this invention two —Si(R²⁴)₃ groups are present in the compounds of formula I, and said —Si(R²⁴)₃ groups are independently selected from the group consisting of: —Si(CH₃)₃ and —Si(CH₂CH₃)₂CH₃ and R¹⁰ is selected from the group consisting of 1A to 42A.

In another embodiment of this invention three —Si(R²⁴)₃ groups are present in the compounds of formula I, and said —Si(R²⁴)₃ groups are independently selected from the group consisting of: —Si(CH₃)₃ and —Si(CH₂CH₃)₂CH₃ and R¹⁰ is selected from the group consisting of 1A to 42A.

In another embodiment of this invention one —Si(R²⁴)₃ group is present in the compounds of formula I, and said —Si(R²⁴)₃ group is —Si(CH₃)₃ and R¹⁰ is selected from the group consisting of 1A to 42A.

In another embodiment of this invention two —Si(R²⁴)₃ groups are present in the compounds of formula I, and said —Si(R²⁴)₃ groups are —Si(CH₃)₃ and R¹⁰ is selected from the group consisting of 1A to 42A.

In another embodiment of this invention three —Si(R²⁴)₃ groups are present in the compounds of formula I, and said —Si(R²⁴)₃ groups are —Si(CH₃)₃ and R¹⁰ is selected from the group consisting of 1A to 42A.

In another embodiment of this invention one group selected from the group consisting of: —SF₅, —OSF₅, —Si(CH₃)₃, —Si(CH₃)₂phenyl, and —Si(CH₂CH₃)₂CH₃) is present in the compounds of formula I, and R¹⁰ is selected from the group consisting of 1A to 42A.

In another embodiment of this invention one group selected from the group consisting of: —SF₅, —OSF₅, —Si(CH₃)₃, and —Si(CH₂CH₃)₂CH₃) is present in the compounds of formula I, and R¹⁰ is selected from the group consisting of 1A to 42A.

In another embodiment of this invention one group selected from the group consisting of: —SF₅, —OSF₅, and —Si(CH₃)₃, is present in the compounds of formula I, and R¹⁰ is selected from the group consisting of 1A to 42A.

In another embodiment of this invention one —SF₅ group is present in the compounds of formula I, and one or two additional groups selected from the group consisting of: —SF₅, —OSF₅, and —Si(R²⁴)₃ (wherein each R²⁴ is independently selected) are also present in the compounds of formula I, and R¹⁰ is selected from the group consisting of 1A to 42A.

In another embodiment of this invention one —SF₅ group is present in the compounds of formula I, and one or two additional groups selected from the group consisting of: —OSF₅, and —Si(R²⁴)₃ (wherein each R²⁴ is independently selected) are also present in the compounds formula I, and R¹⁰ is selected from the group consisting of 1A to 42A.

In another embodiment of this invention one —SF₅ group is present in the compounds of formula I, and one or two additional groups selected from the group consisting of: —SF₅ and —OSF₅ are also present in the compounds of formula I, and R¹⁰ is selected from the group consisting of 1A to 42A.

In another embodiment of this invention one —OSF₅ group is present in the compounds of formula I, and one or two additional groups selected from the group consisting of: —SF₅, —OSF₅, and —Si(R²⁴)₃ (wherein each R²⁴ is independently selected) are also present in the compounds of formula I, and R¹⁰ is selected from the group consisting of 1A to 42A.

In another embodiment of this invention one —OSF₅ group is present in the compounds of formula I, and one or two additional groups selected from the group consisting of: —SF₅ and —Si(R²⁴)₃ (wherein each R²⁴ is independently selected) are also present in the compounds of formula I, and R¹⁰ is selected from the group consisting of 1A to 42A.

In another embodiment of this invention one —OSF₅ group is present in the compounds of formula I, and one or two additional groups selected from the group consisting of: —SF₅ and —OSF₅ are also present in the compounds of formula I, and R¹⁰ is selected from the group consisting of 1A to 42A.

In another embodiment of this invention one —Si(R²⁴)₃ (wherein each R²⁴ is independently selected) group is present in the compounds of formula I, and one or two additional groups selected from the group consisting of: —SF₅, —OSF₅, and —Si(R²⁴)₃ (wherein each R²⁴ is independently selected) are also present in the compounds of formula I, and R¹⁰ is selected from the group consisting of 1A to 42A.

In another embodiment of this invention one —Si(R²⁴)₃ (wherein each R¹⁵ is independently selected) group is present in the compounds of formula I, and one or two additional groups selected from the group consisting of: —SF₅ and —OSF₅ are also present in the compounds of formula I, and R¹⁰ is selected from the group consisting of 1A to 42A.

In another embodiment of this invention at least one group selected from the group consisting of: —SF₅, —OSF₅, and —Si(R²⁴)₃ (wherein each R²⁴ is independently selected from the group consisting of alkyl (e.g., methyl and ethyl) and aryl (e.g., phenyl)) is present in the compounds of formula I, and R¹⁰ is selected from the group consisting of 1A to 42A.

In another embodiment of this invention at least one group selected from the group consisting of: —SF₅, —OSF₅, and —Si(R²⁴)₃ (wherein each R²⁴ is independently selected from the group consisting of alkyl (e.g., methyl and ethyl) and phenyl) is present in the compounds of formula I, and R¹⁰ is selected from the group consisting of 1A to 42A.

In another embodiment of this invention at least one group selected from the group consisting of: —SF₅, —OSF₅, and —Si(R²⁴)₃ (wherein each R²⁴ is independently selected from the group consisting of methyl, ethyl and phenyl) is present in the compounds of formula I, and R¹⁰ is selected from the group consisting of 1A to 42A.

In another embodiment of this invention at least one group selected from the group consisting of: —SF₅, —OSF₅, —Si(CH₃)₃, —Si(CH₃)₂phenyl, and —Si(CH₂CH₃)₂CH₃) is present in the compounds of formula I, and R¹⁰ is selected from the group consisting of 1A to 42A.

In another embodiment of this invention at least one group selected from the group consisting of: —SF₅, —OSF₅, and —Si(CH₃)₃ is present in the compounds of formula I, and R¹⁰ is selected from the group consisting of 1A to 42A.

In another embodiment of this invention one group selected from the group consisting of: —SF₅, —OSF₅, and —Si(R²⁴)₃ (wherein each R²⁴ is independently selected) is present in the compounds of formula I, and R¹⁰ is selected from the group consisting of 1A to 42A.

In another embodiment of this invention one group selected from the group consisting of: —SF₅, —OSF₅, and —Si(R²⁴)₃ (wherein each R²⁴ is independently selected from the group consisting of alkyl (e.g., methyl and ethyl) and aryl (e.g., phenyl)) is present in the compounds of formula I, and R¹⁰ is selected from the group consisting of 1A to 42A.

In another embodiment of this invention one group selected from the group consisting of: —SF₅, —OSF₅, and —Si(R²⁴)₃ (wherein each R²⁴ is independently selected from the group consisting of alkyl (e.g., methyl and ethyl) and phenyl) is present in the compounds of formula I, and R¹⁰ is selected from the group consisting of 1A to 42A.

In another embodiment of this invention one group selected from the group consisting of: —SF₅, —OSF₅, and —Si(R²⁴)₃ (wherein each R²⁴ is independently selected from the group consisting of methyl, ethyl and phenyl) is present in the compounds of formula I, and R¹⁰ is selected from the group consisting of 1A to 42A.

In another embodiment of this invention one group selected from the group consisting of: —SF₅, —OSF₅, —Si(CH₃)₃, —Si(CH₃)₂phenyl, and —Si(CH₂CH₃)₂CH₃) is present in the compounds of formula I, and R¹⁰ is selected from the group consisting of 1A to 42A.

In another embodiment of this invention one group selected from the group consisting of: —SF₅, —OSF₅, —Si(CH₃)₃, and —Si(CH₂CH₃)₂CH₃) is present in the compounds of formula I, and R¹⁰ is selected from the group consisting of 1A to 42A.

In another embodiment of this invention one group selected from the group consisting of: —SF₅, —OSF₅, and —Si(CH₃)₃, is present in the compounds of formula I, and R¹⁰ is selected from the group consisting of 1A to 42A.

In another embodiment of this invention two groups selected from the group consisting of: —SF₅, —OSF₅, and —Si(R²⁴)₃ (wherein each R²⁴ is independently selected) are present in the compounds of formula I, and R¹⁰ is selected from the group consisting of 1A to 42A.

In another embodiment of this invention two groups independently selected from the group consisting of: —SF₅, —OSF₅, and —Si(R²⁴)₃ (wherein each R²⁴ is independently selected from the group consisting of alkyl (e.g., methyl and ethyl) and aryl (e.g., phenyl)) are present in the compounds of formula I, and R¹⁰ is selected from the group consisting of 1A to 42A.

In another embodiment of this invention two groups independently selected from the group consisting of: —SF₅, —OSF₅, and —Si(R²⁴)₃ (wherein each R²⁴ is independently selected from the group consisting of alkyl (e.g., methyl and ethyl) and phenyl) are present in the compounds of formula I, and R¹⁰ is selected from the group consisting of 1A to 42A.

In another embodiment of this invention two groups selected from the group consisting of: —SF₅, —OSF₅, and —Si(R²⁴)₃ (wherein each R²⁴ is independently selected from the group consisting of methyl, ethyl and phenyl) are present in the compounds of formula I, and R¹⁰ is selected from the group consisting of 1A to 42A.

In another embodiment of this invention two groups independently selected from the group consisting of: —SF₅, —OSF₅, —Si(CH₃)₃, —Si(CH₃)₂phenyl, and —Si(CH₂CH₃)₂CH₃) is present in the compounds of formula I, and R¹⁰ is selected from the group consisting of 1A to 42A.

In another embodiment of this invention two groups independently selected from the group consisting of: —SF₅, —OSF₅, —Si(CH₃)₃, and —Si(CH₂CH₃)₂CH₃) are present in the compounds of formula I, and R¹⁰ is selected from the group consisting of 1A to 42A.

In another embodiment of this invention two groups independently selected from the group consisting of: —SF₅, —OSF₅, and —Si(CH₃)₃ are present in the compounds of formula I, and R¹⁰ is selected from the group consisting of 1A to 42A.

In another embodiment of this invention three groups selected from the group consisting of: —SF₅, —OSF₅, and —Si(R²⁴)₃ (wherein each R²⁴ is independently selected) are present in the compounds of formula I, and R¹⁰ is selected from the group consisting of 1A to 42A.

In another embodiment of this invention three groups independently selected from the group consisting of: —SF₅, —OSF₅, and —Si(R²⁴)₃ (wherein each R²⁴ is independently selected from the group consisting of alkyl (e.g., methyl and ethyl) and aryl (e.g., phenyl)) are present in the compounds of formula I, and R¹⁰ is selected from the group consisting of 1A to 42A.

In another embodiment of this invention three groups independently selected from the group consisting of: —SF₅, —OSF₅, and —Si(R²⁴)₃ (wherein each R²⁴ is independently selected from the group consisting of alkyl (e.g., methyl and ethyl) and phenyl) are present in the compounds of formula I, and R¹⁰ is selected from the group consisting of 1A to 42A.

In another embodiment of this invention three groups selected from the group consisting of: —SF₅, —OSF₅, and —Si(R²⁴)₃ (wherein each R²⁴ is independently selected from the group consisting of methyl, ethyl and phenyl) are present in the compounds of formula I, and R¹⁰ is selected from the group consisting of 1A to 42A.

In another embodiment of this invention three groups independently selected from the group consisting of: —SF₅, —OSF₅, —Si(CH₃)₃, —Si(CH₃)₂phenyl, and —Si(CH₂CH₃)₂CH₃) is present in the compounds of formula I, and R¹⁰ is selected from the group consisting of 1A to 42A.

In another embodiment of this invention three groups independently selected from the group consisting of: —SF₅, —OSF₅, —Si(CH₃)₃, and —Si(CH₂CH₃)₂CH₃) are present in the compounds of formula 1, and R¹⁰ is selected from the group consisting of 1A to 42A.

In another embodiment of this invention three groups independently selected from the group consisting of: —SF₅, —OSF₅, and —Si(CH₃)₃ are present in the compounds of formula I, and R¹⁰ is selected from the group consisting of 1A to 42A.

In another embodiment of this invention at least one group selected from the group consisting of: —SF₅, —OSF₅, and —Si(R²⁴)₃ (wherein each R²⁴ is the same or different alkyl group) is present in the compounds of formula 1, and R¹⁰ is selected from the group consisting of 1A to 42A.

In another embodiment of this invention at least one group selected from the group consisting of: —SF₅, —OSF₅, and —Si(R²⁴)₃ (wherein each R²⁴ is independently selected from the group consisting of methyl and ethyl) is present in the compounds of formula I, and R¹⁰ is selected from the group consisting of 1A to 42A.

Other embodiments of this invention are directed to any one of the embodiments above directed to the groups —SF₅, —OSF₅, or —Si(R²⁴)₃ wherein R¹⁰ is 35A.

Another embodiment to this invention is directed to any one of the embodiments above directed to the compounds of formula I wherein:
R¹ is optionally substituted with 1-5 substituents independently selected from the group consisting of halo, —CN, —NH₂, —NH(alkyl), —N(alkyl)₂, hydroxy and alkoxy;
R² is optionally substituted with 1-5 substituents independently selected from the group consisting of halo, —CN, —NH₂, —NH(alkyl), —N(alkyl)₂, hydroxy and alkoxy;
or, when R¹ and R² are joined together to form a C5-C8 cycloalkyl or a 5-8 membered heterocyclyl moiety, each of said cycloalkyl or heterocyclyl moiety is optionally substituted with 1-5 substituents independently selected from the group consisting of halo, —CN, —NH₂, —NH(alkyl), —N(alkyl)₂, hydroxy and alkoxy groups;
each of said alkyl, aryl, heteroaryl, cycloalkyl, heterocyclyl, arylalkyl-, alkylaryl-, cycloalkylalkyl-, heteroarylalkyl- and heterocyclyalkyl- R⁵, R⁶ and R⁷ groups are optionally substituted with 1-5 substituents independently selected from the group consisting of: halo, —CN, —NH₂, —NH(alkyl), —N(alkyl)₂, hydroxy, alkoxy, -aryl and -heteroaryl groups;
or when R⁶ and R⁷ are joined together to form a carbocyclic spirocyclic moiety or a heterocyclic spirocyclic moiety, each of said carbocyclic spirocyclic moiety and heterocyclic spirocyclic moiety is: (i) optionally substituted with 1-4 substituents independently selected from the group consisting of halo, alkyl, —CN, —NH₂, —NH(alkyl), —N(alkyl)₂, hydroxy, alkoxy, aryl and heteroaryl groups, or is (ii) optionally fused with an aryl, heteroaryl, cycloalkyl or heterocycloalkyl ring, wherein each of said carbocyclic spirocyclic moiety, heterocyclic spirocyclic moiety, aryl, heteroaryl, cycloalkyl and heterocycloalkyl ring is optionally substituted with 1-4 substituents independently selected from the group consisting of halo, alkyl, —CN, —NH₂, —NH(alkyl), —N(alkyl)₂, hydroxy, alkoxy, aryl and heteroaryl groups;
each of said alkyl, cycloalkyl, aryl and heteroaryl R⁸ group is optionally substituted with 1-3 substituents independently selected from the group consisting of halo, alkyl, —CN, —NH₂, —NH(alkyl), —N(alkyl)₂, hydroxy and alkoxy groups;
each R¹⁰ group (excluding the bond) is optionally substituted with 1-3 substituents independently selected from the group consisting of halo, alkyl, —CN, —NH₂, —NH(alkyl), —N(alkyl)₂, hydroxy and alkoxy groups; and

each of said alkyl, aryl, heteroaryl, heterocyclyl, arylalkyl-, alkylaryl-, heteroarylalkyl- and heterocyclyalkyl R⁹ group is optionally substituted with 1-3 substituents independently selected from the group consisting of halo, alkyl, —CN, —NH₂, —NH(alkyl), —N(alkyl)₂, hydroxy and alkoxy groups.

In another embodiment the compounds of formula I are compounds of formula II:

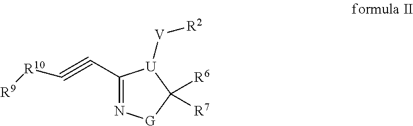

wherein G, U, V, R², R⁶, R⁷, R⁹, and R¹⁰ are as defined for formula I. In one embodiment of the compounds of formula II, there are 1 to 3 (in one example there is one, in another example there are 2, and in another example there are three) groups selected from the group consisting of —SF₅, —OSF₅ and —Si(R²⁴)₃ present on either R⁶ or R⁷ (and in one example on R⁶, and in another example R⁷, and in another example distributed between R⁶ and R⁷ when there is more than one of said groups). In one embodiment of the compounds of formula III, there are 1 to 3 (in one example there is one, in another example there are 2, and in another example there are three) groups selected from the group consisting of —SF₅ and —OSF₅ present on either R⁶ or R⁷ (and in one example on R⁶, and in another example R⁷, and in another example distributed between R⁶ and R⁷ when there is more than one of said groups).

In another embodiment of this invention, R¹ and R² are joined together to form a 5 to 8 membered cycloalkyl ring, and said ring is substituted with a group selected from the group consisting of —SF₅, —OSF₅ and —Si(R²⁴)₃. In another embodiment said ring is substituted with a group selected from the group consisting of —SF₅ and —OSF₅. In another embodiment said ring is substituted with a —SF₅ group. In another embodiment said ring is substituted with an —OSF₅ group. In another embodiment said ring is substituted with a —Si(R²⁴)₃ group. Examples of the —Si(R²⁴)₃ group in the embodiments above include groups wherein each R²⁴ is the same or different alkyl group (e.g., methyl and ethyl). Thus, —Si(CH₃)₃ and —Si(CH₂CH₃)₂CH₃) are examples of the —Si(R²⁴)₃ group in the above embodiments. And in one example the —Si(R²⁴)₃ group is —Si(CH₃)₃.

In another embodiment of this invention, R¹ and R² are joined together to form a 5 to 8 membered heterocyclyl ring, and said ring is substituted with a group selected from the group consisting of —SF₅, —OSF₅ and —Si(R²⁴)₃. In another embodiment said ring is substituted with a group selected from the group consisting of —SF₅ and —OSF₅. In another embodiment said ring is substituted with a —SF₅ group. In another embodiment said ring is substituted with an —OSF₅ group. In another embodiment said ring is substituted with a —Si(R²⁴)₃ group. Examples of the —Si(R²⁴)₃ group in the embodiments above include groups wherein each R²⁴ is the same or different alkyl group (e.g., methyl and ethyl). Thus, —Si(CH₃)₃ and —Si(CH₂CH₃)₂CH₃) are examples of the —Si(R²⁴)₃ group in the above embodiments. And in one example the —Si(R²⁴)₃ group is —Si(CH₃)₃.

In another embodiment of this invention, R⁶ and R⁷ are joined together to form a carbocyclic spirocyclic moiety, and said spirocyclic moiety is substituted with a group selected from the group consisting of —SF₅, —OSF₅ and —Si(R²⁴)₃. In another embodiment said spirocyclic moiety is substituted with a group selected from the group consisting of —SF₅ and —OSF₅. In another embodiment said spirocyclic moiety is substituted with a —SF₅ group. In another embodiment said spirocyclic moiety is substituted with an —OSF₅ group. In another embodiment said spirocyclic moiety is substituted with a —Si(R²⁴)₃ group. Examples of the —Si(R²⁴)₃) group in the embodiments above include groups wherein each R²⁴ is the same or different alkyl group (e.g., methyl and ethyl). Thus, —Si(CH₃)₃ and —Si(CH₂CH₃)₂CH₃) are examples of the —Si(R²⁴)₃ group in the above embodiments. And in one example the —Si(R²⁴)₃ group is —Si(CH₃)₃.

In another embodiment of this invention, R⁶ and R⁷ are joined together to form a heterocyclic spirocyclic moiety, and said spirocyclic moiety is substituted with a group selected from the group consisting of —SF₅, —OSF₅ and —Si(R²⁴)₃. In another embodiment said spirocyclic moiety is substituted with a group selected from the group consisting of —SF₅ and —OSF₅. In another embodiment said spirocyclic moiety is substituted with a —SF₅ group. In another embodiment said spirocyclic moiety is substituted with an —OSF₅ group. In another embodiment said spirocyclic moiety is substituted with a —Si(R²⁴)₃ group. Examples of the —Si(R²⁴)₃ group in the embodiments above include groups wherein each R²⁴ is the same or different alkyl group (e.g., methyl and ethyl). Thus, —Si(CH₃)₃ and —Si(CH₂CH₃)₂CH₃) are examples of the —Si(R²⁴)₃ group in the above embodiments. And in one example the —Si(R²⁴)₃ group is —Si(CH₃)₃.

In another embodiment the compounds of formula I are compounds of formula III:

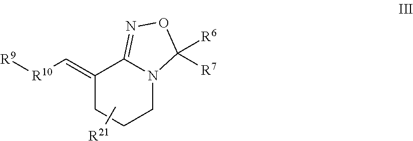

wherein R⁶, R⁷, R⁹, R¹⁰ and R²¹ are as defined in formula I. In one embodiment of the compounds of formula III, there are 1 to 3 (in one example there is one, in another example there are 2, and in another example there are three) groups selected from the group consisting of —SF₅, —OSF₅ and —Si(R²⁴)₃ present on either R⁶ or R⁷ (and in one example on R⁶, and in another example R⁷, and in another example distributed between R⁶ and R⁷ when there is more than one of said groups). In one embodiment of the compounds of formula III, there are 1 to 3 (in one example there is one, in another example there are 2, and in another example there are three) groups selected from the group consisting of —SF₅ and —OSF₅ present on either R⁶ or R⁷ (and in one example on R⁶, and in another example R⁷, and in another example distributed between R⁶ and R⁷ when there is more than one of said groups). In another embodiment of this invention R²¹ in formula III is selected from the group consisting of —SF₅, —OSF₅ and —Si(R²⁴)₃. In another embodiment of this invention R²¹ in formula III is selected from the group consisting of —SF₅ and —OSF₅. In another embodiment of this invention R²¹ in formula III is —SF₅. In another embodiment of this invention R²¹ in formula III is —OSF₅. In another embodiment of this invention R²¹ in formula III is —Si(R²⁴)₃. Examples of the —Si(R²⁴)₃ group in the embodiments above include groups wherein each R²⁴ is the same or different alkyl group (e.g., methyl and ethyl). Thus, —Si(CH₃)₃ and —Si(CH₂CH₃)₂CH₃) are examples of the —Si(R²⁴)₃ group in the above embodiments. And in one example the —Si(R²⁴)₃ group is —Si(CH₃)₃.

In another embodiment the compounds of formula I are compounds of formula IV:

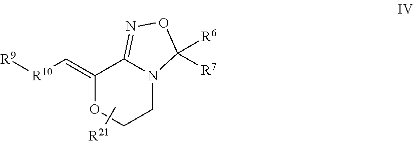

wherein R⁶, R⁷, R⁹, R¹⁰ and R²¹ are as defined in formula I. In one embodiment of the compounds of formula IV, there are 1 to 3 (in one example there is one, in another example there are 2, and in another example there are three) groups selected from the group consisting of —SF₅, —OSF₅ and —Si(R²⁴)₃ present on either R⁶ or R⁷ (and in one example on R⁶, and in another example R⁷, and in another example distributed between R⁶ and R⁷ when there is more than one of said groups). In one embodiment of the compounds of formula IV, there are 1 to 3 (in one example there is one, in another example there are 2, and in another example there are three) groups selected from the group consisting of —SF₅ and —OSF₅ present on either R⁶ or R⁷ (and in one example on R⁶, and in another example R⁷, and in another example distributed between R⁶ and R⁷ when there is more than one of said groups). In another embodiment of this invention R²¹ in formula IV is selected from the group consisting of —SF₅, —OSF₅ and —Si(R²⁴)₃. In another embodiment of this invention R²¹ in formula IV is selected from the group consisting of —SF₅ and —OSF₅. In another embodiment of this invention R²¹ in formula IV is —SF₅. In another embodiment of this invention R²¹ in formula IV is —OSF₅. In another embodiment of this invention R²¹ in formula IV is —Si(R²⁴)₃. Examples of the —Si(R²⁴)₃ group in the embodiments above include groups wherein each R²⁴ is the same or different alkyl group (e.g., methyl and ethyl). Thus, —Si(CH₃)₃ and —Si(CH₂CH₃)₂CH₃) are examples of the —Si(R²⁴)₃ group in the above embodiments. And in one example the —Si(R²⁴)₃ group is —Si(CH₃)₃.

In another embodiment the compounds of formula I are compounds of formula V:

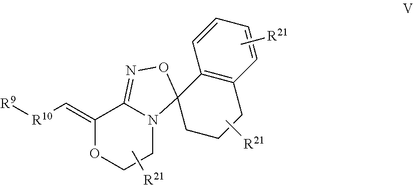

wherein R⁶, R⁷, R⁹, R¹⁰ and R²¹ are as defined in formula I. In one embodiment of the compounds of formula V, there are 1 to 3 groups (in one example there is one, in another example there are two, and in anther example there are three) independently selected from the group consisting of —SF₅, —OSF₅ and —Si(R²⁴)₃ present on the Spiro fused phenylcyclohexyl-ring (i.e., the spiro tetrahydro-naphthalene ring), and said groups can be one of or both of the R²¹ groups shown in formula V, or said group can be in addition to the R²¹ groups shown in formula V when said R²¹ groups are other than a —SF₅, —OSF₅ and —Si(R²⁴)₃ group. In another embodiment of the compounds of formula V, there are 1 to 3 groups (in one example there is one, in another example there are two, and in anther example there are three) independently selected from the group consisting of —SF₅ and —OSF₅ present on the spiro fused phenylcyclohexyl-ring (i.e., the spiro tetrahydronaphthalene ring), and said groups can be one of or both of the R²¹ groups shown in formula V, or said group can be in addition to the R²¹ groups shown in formula V when said R²¹ groups are other than —SF₅ and —OSF₅ groups. In another embodiment of this invention R²¹ in the formula V Moiety A:

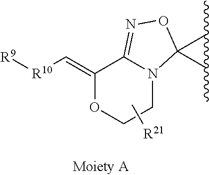

is selected from the group consisting of —SF₅, —OSF₅ and —Si(R²⁴)₃. In another embodiment of this invention R²¹ in the formula V Moiety A is selected from the group consisting of —SF₅ and —OSF₅. In another embodiment of this invention R²¹ in the formula V Moiety A is —SF₅. In another embodiment of this invention R²¹ in formula V Moiety A is —OSF₅. In another embodiment of this invention R²¹ in the formula V Moiety A is —Si(R²⁴)₃. Examples of the —Si(R²⁴)₃ group in the embodiments above include groups wherein each R²⁴ is the same or different alkyl group (e.g., methyl and ethyl). Thus, —Si(CH₃)₃ and —Si(CH₂CH₃)₂CH₃) are examples of the —Si(R²⁴)₃ group in the above embodiments. And in one example the —Si(R²⁴)₃ group is —Si(CH₃)₃.

Examples of formula V include:

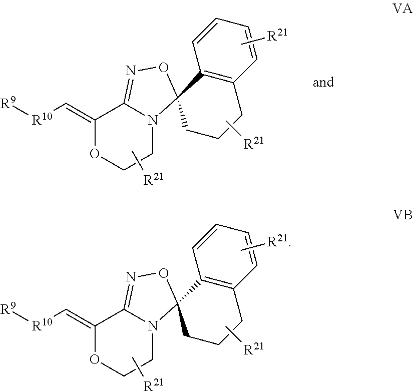

In another embodiment the compounds of formula I are compounds of formula VI:

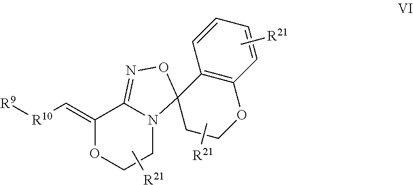

wherein R⁶, R⁷, R⁹, R¹⁰ and R²¹ are as defined in formula I. In one embodiment of the compounds of formula VI, there are 1 to 3 groups (in one example there is one, in another example there are two, and in another example there are three) independently selected from the group consisting of —SF₅, —OSF₅ and —Si(R²⁴)₃ present on the spiro fused phenyltetrahydropyranyl-ring (i.e., the spiro chroman ring), and said groups can be one of or both of the R²¹ groups shown in formula VI, or said group can be in addition to the R²¹ groups shown in formula VI when said R²¹ groups are other than a —SF₅, —OSF₅ and —Si(R²⁴)₃ group. In another embodiment of the compounds of formula VI, there are 1 to 3 groups (in one example there is one, in another example there are two, and in anther example there are three) independently selected from the group consisting of —SF₅ and —OSF₅ present on the spiro fused phenyltetrahydropyranyl-ring (i.e., the spiro chroman ring), and said groups can be one of or both of the R²¹ groups shown in formula VI, or said group can be in addition to the R²¹ groups shown in formula VI when said R²¹ groups are other than —SF₅ and —OSF₅ groups. In another embodiment of this invention R²¹ in the formula VI Moiety A:

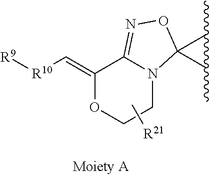

is selected from the group consisting of —SF₅, —OSF₅ and —Si(R²⁴)₃. In another embodiment of this invention R²¹ in the formula VI Moiety A is selected from the group consisting of —SF₅ and —OSF₅. In another embodiment of this invention R²¹ in the formula VI Moiety A is —SF₅. In another embodiment of this invention R²¹ in formula VI Moiety A is —OSF₅. In another embodiment of this invention R²¹ in the formula VI Moiety A is —Si(R²⁴)₃. Examples of the —Si(R²⁴)₃ group in the embodiments above include groups wherein each R²⁴ is the same or different alkyl group (e.g., methyl and ethyl). Thus, —Si(CH₃)₃ and —Si(CH₂CH₃)₂CH₃) are examples of the —Si(R²⁴)₃ group in the above embodiments. And in one example the —Si(R²⁴)₃ group is —Si(CH₃)₃.

In another embodiment the compounds of formula I are compounds of formula VII:

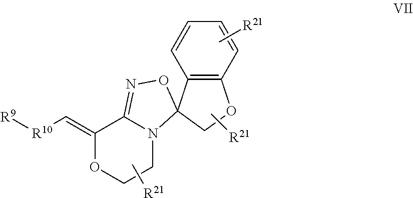

wherein R⁶, R⁷, R⁹, R¹⁰ and R²¹ are as defined in formula I. In one embodiment of the compounds of formula VII, there are 1 to 3 groups (in one example there is one, in another example there are two, and in anther example there are three) independently selected from the group consisting of —SF₅, —OSF₅ and —Si(R²⁴)₃ present on the spiro fused phenyltetrahydrofuranyl-ring (i.e., the spiro dihydrobenzofuran ring), and said groups can be one of or both of the R²¹ groups shown in formula VII, or said group can be in addition to the R²¹ groups shown in formula VII when said R²¹ groups are other than a —SF₅, —OSF₅ and —Si(R²⁴)₃ group. In another embodiment of the compounds of formula VII, there are 1 to 3 groups (in one example there is one, in another example there are two, and in anther example there are three) independently selected from the group consisting of —SF₅ and —OSF₅ present on the spiro fused phenyltetrahydrofuranyl-ring (i.e., the spiro dihydrobenzofuran ring), and said groups can be one of or both of the R²¹ groups shown in formula VI, or said group can be in addition to the R²¹ groups shown in formula VI when said R²¹ groups are other than —SF₅ and —OSF₅ groups. In another embodiment of this invention R²¹ in the formula VII Moiety A:

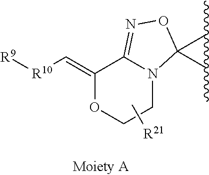

is selected from the group consisting of —SF₅, —OSF₅ and —Si(R²⁴)₃. In another embodiment of this invention R²¹ in the formula VII Moiety A is selected from the group consisting of —SF₅ and —OSF₅. In another embodiment of this invention R²¹ in the formula VII Moiety A is —SF₅. In another embodiment of this invention R²¹ in formula VII Moiety A is —OSF₅. In another embodiment of this invention R²¹ in the formula VII Moiety A is —Si(R²⁴)₃. Examples of the —Si(R²⁴)₃ group in the embodiments above include groups wherein each R²⁴ is the same or different alkyl group (e.g., methyl and ethyl). Thus, —Si(CH₃)₃ and —Si(CH₂CH₃)₂CH₃) are examples of the —Si(R²⁴)₃ group in the above embodiments. And in one example the —Si(R²⁴)₃ group is —Si(CH₃)₃.

In another embodiment the compounds of formula I are compounds of formula VIII:

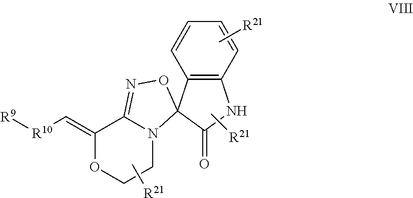

wherein R⁶, R⁷, R⁹, R¹⁰ and R²¹ are as defined in formula I. In one embodiment of the compounds of formula VIII, there are 1 to 3 groups (in one example there is one, in another example there are two, and in anther example there are three) independently selected from the group consisting of —SF₅, —OSF₅ and —Si(R²⁴)₃ present on the spiro fused phenylpyrrolidinyl-ring (i.e., the spiro Indoline ring), and said groups can be one of or both of the R²¹ groups shown in formula VII, or said group can be in addition to the R²¹ groups shown in formula VII when said R²¹ groups are other than a —SF₅, —OSF₅ and —Si(R²⁴)₃ group. In another embodiment of the compounds of formula VII, there are 1 to 3 groups (in one example there is one, in another example there are two, and in anther example there are three) independently selected from the group consisting of —SF₅ and —OSF₅ present on the spiro fused phenyloyrrolidinyl-ring (i.e., the spiro Indoline ring), and said groups can be one of or both of the R²¹ groups shown in formula VI, or said group can be in addition to the R²¹ groups shown in formula VI when said R²¹ groups are other than —SF₅ and —OSF₅ groups. In another embodiment of this invention R²¹ in the formula VIII Moiety A:

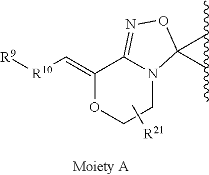

is selected from the group consisting of —SF₅, —OSF₅ and —Si(R²⁴)₃. In another embodiment of this invention R²¹ in the formula VIII Moiety A is selected from the group consisting of —SF₅ and —OSF₅. In another embodiment of this invention R²¹ in the formula VIII Moiety A is —SF₅. In another embodiment of this invention R²¹ in formula VIII Moiety A is —OSF₅. In another embodiment of this invention R²¹ in the formula VIII Moiety A is —Si(R²⁴)₃. Examples of the —Si(R²⁴)₃ group in the embodiments above include groups wherein each R²⁴ is the same or different alkyl group (e.g., methyl and ethyl). Thus, —Si(CH₃)₃ and —Si(CH₂CH₃)₂CH₃) are examples of the —Si(R²⁴)₃ group in the above embodiments. And in one example the —Si(R²⁴)₃ group is —Si(CH₃)₃.

In another embodiment the compounds of formula I are compounds of formula IX:

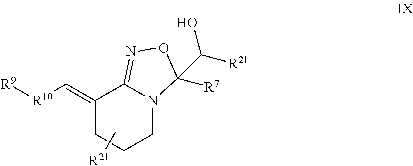

wherein R⁶, R⁷, R⁹, R¹⁰ and R²¹ are as defined in formula I. In one embodiment of the compounds of formula IX, there are 1 to 3 (in one example there is one, in another example there are 2, and in another example there are three) groups selected from the group consisting of —SF₅, —OSF₅ and —Si(R²⁴)₃ present on either the R⁶—CH(OH)R²¹ group or on the R⁷ group (and in one example on R⁶, and in another example R⁷, and in another example distributed between R⁶ and R⁷ when there is more than one of said groups), and when present on the R⁶ group said H (of said CH moiety) on said R⁶ group can be replaced with one of the of —SF₅, —OSF₅ or —Si(R²⁴)₃ groups, and/or the R²¹ group on said R⁶ group can be one of the of —SF₅, —OSF₅ or —Si(R²⁴)₃ groups. In another embodiment of the compounds of formula IX, there are 1 to 3 (in one example there is one, in another example there are 2, and in another example there are three) groups selected from the group consisting of —SF₅ and —OSF₅ present on either the R⁶—CH(OH)R²¹ group or on the R⁷ group (and in one example on R⁶, and in another example R⁷, and in another example distributed between R⁶ and R⁷ when there is more than one of said groups), and when present on the R⁶ group said H (of said CH moiety) on said R⁶ group can be replaced with one of the of —SF₅ or —OSF₅ groups, and/or the R²¹ group on said R⁶ group can be one of the of —SF₅ or —OSF₅ groups. In another embodiment of this invention R²¹ in the formula IX Moiety B:

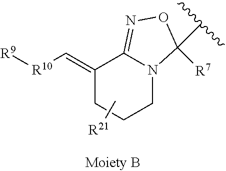

is selected from the group consisting of —SF₅, —OSF₅ and —Si(R²⁴)₃. In another embodiment of this invention R²¹ in the formula IX Moiety B is selected from the group consisting of —SF₅ and —OSF₅. In another embodiment of this invention R²¹ in the formula IX Moiety B is —SF₅. In another embodiment of this invention R²¹ in formula IX Moiety B is —OSF₅. In another embodiment of this invention R²¹ in the formula IX Moiety B is —Si(R²⁴)₃. Examples of the —Si(R²⁴)₃ group in the embodiments above include groups wherein each R²⁴ is the same or different alkyl group (e.g., methyl and ethyl). Thus, —Si(CH₃)₃ and —Si(CH₂CH₃)₂CH₃) are examples of the —Si(R²⁴)₃ group in the above embodiments. And in one example the —Si(R²⁴)₃ group is —Si(CH₃)₃.

In another embodiment the compounds of formula I are compounds of formula X:

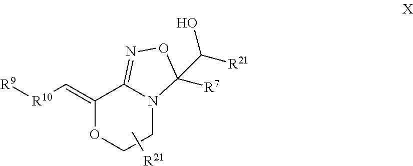

wherein R⁶, R⁷, R⁹, R¹⁰ and R²¹ are as defined in formula I. In one embodiment of the compounds of formula X, there are 1 to 3 (in one example there is one, in another example there are 2, and in another example there are three) groups selected from the group consisting of —SF₅, —OSF₅ and —Si(R²⁴)₃ present on either the R⁶—CH(OH)R²¹ group or on the R⁷ group (and in one example on R⁶, and in another example R⁷, and in another example distributed between R⁶ and R⁷ when there is more than one of said groups), and when present on the R⁶ group said H (of said CH moiety) on said R⁶ group can be replaced with one of the of —SF₅, —OSF₅ or —Si(R²⁴)₃ groups, and/or the R²¹ group on said R⁶ group can be one of the of —SF₅, —OSF₅ or —Si(R²⁴)₃ groups. In another embodiment of the compounds of formula X, there are 1 to 3 (in one example there is one, in another example there are 2, and in another example there are three) groups selected from the group consisting of —SF₅ and —OSF₅ present on either the R⁶—CH(OH)R²¹ group or on the R⁷ group (and in one example on R⁶, and in another example R⁷, and in another example distributed between R⁶ and R⁷ when there is more than one of said groups), and when present on the R⁶ group said H (of said CH moiety) on said R⁶ group can be replaced with one of the of —SF₅ or —OSF₅ groups, and/or the R²¹ group on said R⁶ group can be one of the of —SF₅ or —OSF₅ groups. In another embodiment of this invention R²¹ in the formula X Moiety C:

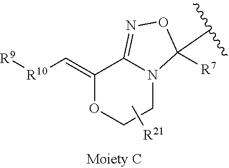

is selected from the group consisting of —SF₅, —OSF₅ and —Si(R²⁴)₃. In another embodiment of this invention R²¹ in the formula formula X Moiety C is selected from the group consisting of —SF₅ and —OSF₅. In another embodiment of this invention R²¹ in the formula formula X Moiety C is —SF₅. In another embodiment of this invention R²¹ in formula formula X Moiety C is —OSF₅. In another embodiment of this invention R²¹ in the formula formula X Moiety C is —Si(R²⁴)₃. Examples of the —Si(R²⁴)₃ group in the embodiments above include groups wherein each R²⁴ is the same or different alkyl group (e.g., methyl and ethyl). Thus, —Si(CH₃)₃ and —Si(CH₂CH₃)₂CH₃) are examples of the —Si(R²⁴)₃ group in the above embodiments. And in one example the —Si(R²⁴)₃ group is —Si(CH₃)₃.

Those skilled in the art will appreciate that for the compounds of the invention:

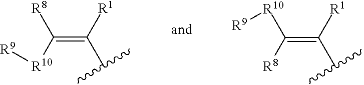

are isomers

Those skilled in the art will appreciate that in the compounds of the invention R⁶ can be:

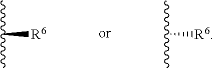

Those skilled in the art will appreciate that in the compounds of the invention R⁷ can be:

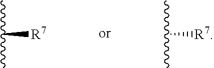

Thus, for example, in embodiments of this invention R⁶ and R⁷ can be:

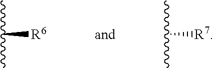

In other embodiments of this invention R⁶ and R⁷ can be:

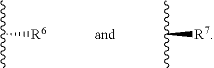

In another embodiment G is O.

In another embodiment G is S.

In another embodiment V is selected from the group consisting of a bond, O, and N(R¹⁴).

In another embodiment V is a bond.

In another embodiment V is —C(O)—.

In another embodiment V is —N(R¹⁴)—.

In another embodiment R⁸ is selected from the group consisting of H, alkyl-, aryl-, arylalkyl-, alkylaryl-, cycloalkyl-, cycloalkylalkyl-, heteroaryl-, heteroarylalkyl-, heterocyclyl- and heterocyclyalkyl-, with each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the R²¹ moieties.

In another embodiment R⁸ is selected from the group consisting of H, alkyl, cycloalkyl, aryl and heteroaryl, with each of said alkyl, cycloalkyl, aryl and heteroaryl being unsubstituted or optionally independently substituted with 1-3 substituents which can be the same or different, each substituent being independently selected from the group consisting of the R²¹ moieties.

In another embodiment R⁸ is selected from the group consisting of H, alkyl, cycloalkyl, aryl and heteroaryl, with each of said alkyl, cycloalkyl, aryl and heteroaryl being unsubstituted or optionally independently substituted with 1-3 substituents which can be the same or different, each substituent being independently selected from the group consisting of halo, alkyl, —CN, —NH₂, —NH(alkyl), —N(alkyl)₂, hydroxy and alkoxy groups.

In another embodiment, R⁸ and R¹⁰, together with the carbon atom to which they are bound, form a C₄-C₇ carbocyclic (e.g., cycloalkyl) ring, or a 4-7 membered heterocyclyl ring, or a C₄-C₇ carbocyclenyl (e.g., cycloalkenyl) ring, or a 4-7 membered heterocyclenyl ring; and wherein said carbocyclic ring, heterocyclyl ring, carbocyclenyl ring, or heterocyclenyl ring is optionally substituted with 1-5 independently selected R²¹ substituents.

In another embodiment, R⁸ and R¹⁰, together with the carbon atom to which they are bound, form a C₄-C₇ carbocyclic (e.g., cycloalkyl) ring; and wherein said carbocyclic ring is optionally substituted with 1-5 independently selected R²¹ substituents.

In another embodiment, R⁸ and R¹⁰, together with the carbon atom to which they are bound, form a 4-7 membered heterocyclyl ring; and wherein said heterocyclyl ring is optionally substituted with 1-5 independently selected R²¹ substituents.

In another embodiment, R⁸ and R¹⁰, together with the carbon atom to which they are bound, form a C₄-C₇ carbocyclenyl (e.g., cycloalkenyl) ring; and wherein said carbocyclenyl ring is optionally substituted with 1-5 independently selected R²¹ substituents.

In another embodiment, R⁸ and R¹⁰, together with the carbon atom to which they are bound, form a 4-7 membered heterocyclenyl ring; and wherein said heterocyclenyl ring is optionally substituted with 1-5 independently selected R²¹ substituents.

In another embodiment, U is C(R⁵).

In another embodiment, U is N.

In another embodiment R¹ is H.

In another embodiment R¹ is halo (e.g., Br).

In another embodiment, R² is H.

In another embodiment, R² is alkyl.

In another embodiment, R² is methyl.

In another embodiment, R² is alkoxyalkyl-.

In another embodiment, R² is 3-methoxypropyl-.

In another embodiment, U is N and R² is 3-methoxypropyl-.

In another embodiment, R² is arylalkyl-.

In another embodiment R² is selected from the group consisting of: (a) alkyl, (b) alkyl substituted with an —OR¹⁵ group (e.g., —Oalkyl, such as, for example, —OCH₃) or a halo group (e.g., Cl), (c) arylalkyl-substituted with an —OR¹⁵ group (e.g., —Oalkyl, such as, for example, —OCH₃) or a halo group (e.g. F), (d) cycloalkyl (e.g., cyclopropyl), (e) heterocycloalkyl (e.g.,

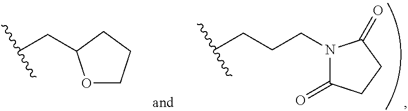

and
(e) cycloalkylalkyl-substituted with a halo substituted alkyl (e.g. wherein the halo substituted alkyl is —CH₂Cl).

In another embodiment R² is selected from the group consisting of: H, —(CH₂)₃OCH₃, —CH₃, —CH₂CH₃, —(CH₂)₂OCH₃, —(CH₂)₂CH(CH₃)₂, —CH₂CH(CH₃)CH₂CH₃, —CH₂CH₂CH₃,

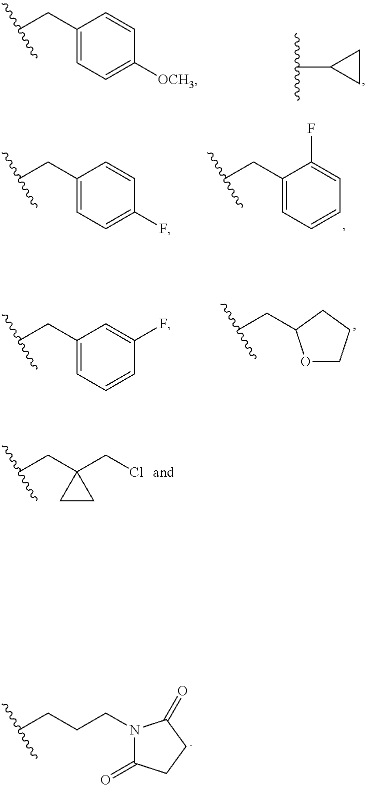

In another embodiment R² is selected from the group consisting of: H, —(CH₂)₃OCH₃, —CH₃, —CH₂CH₃, —(CH₂)₂OCH₃, —(CH₂)₂CH(CH₃)₂, —CH₂CH(CH₃)CH₂CH₃, —CH₂CH₂CH₃,

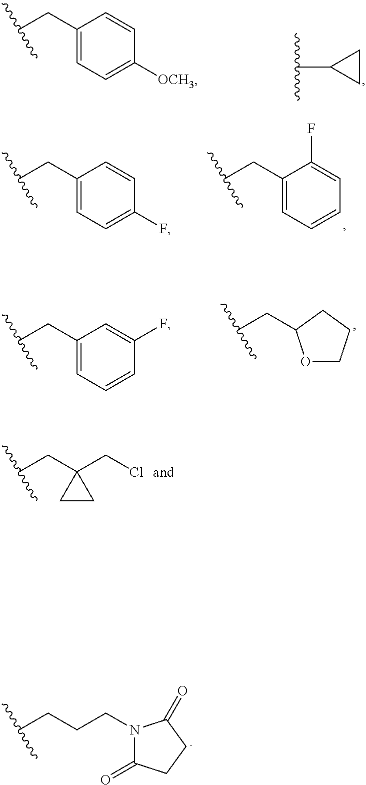

In another embodiment, R² is phenylmethyl-.

In another embodiment, R² is (4-alkoxy)phenylmethyl-.

In another embodiment, R² is (4-methoxy)phenylmethyl-.

In another embodiment, R¹ is H.

In another embodiment, R¹ is alkyl.

In another embodiment, R¹ is methyl.

In another embodiment, R¹ and R² are joined together to form a cyclopentyl ring, which is unsubstituted.

In another embodiment, R¹ and R² are joined together to form a cyclopentyl ring, which is substituted with 1-3 substituents which can be the same or different, each being independently selected from the group consisting of halo, alkyl, —CN, —NH₂, —NH(alkyl), —N(alkyl)₂, hydroxy and alkoxy groups.

In another embodiment, R¹ and R² are joined together to form a cyclohexyl ring, which is unsubstituted.

In another embodiment, R¹ and R² are joined together to form a cyclohexyl ring, which is substituted with 1-3 substituents which can be the same or different, each being independently selected from the group consisting of halo, alkyl, —CN, —NH₂, —NH(alkyl), —N(alkyl)₂, hydroxy and alkoxy groups.

In another embodiment, U is N, and R¹ and R² are joined together to form a piperidinyl ring including the N of U as the nitrogen of said piperidinyl ring, which is unsubstituted.

In another embodiment, U is N, and R¹ and R² are joined together to form a piperidinyl ring including the N of U as the nitrogen of said piperidinyl ring, wherein said piperidinyl ring is substituted with 1-3 substituents which can be the same or different, each being independently selected from the group consisting of halo, alkyl, —CN, —NH₂, —NH(alkyl), —N(alkyl)₂, hydroxy and alkoxy groups.

In another embodiment, U is N, and R¹ and R² are joined together to form a pyrrolidinyl ring including the N of U as the nitrogen of said pyrrolidinyl ring, which is unsubstituted.

In another embodiment, U is N, and R¹ and R² are joined together to form a pyrrolidinyl ring including the N of U as the nitrogen of said pyrrolidinyl ring, wherein said pyrrolidinyl ring is substituted with 1-3 substituents which can be the same or different, each being independently selected from the group consisting of halo, alkyl, —CN, —NH₂, —NH(alkyl), —N(alkyl)₂, hydroxy and alkoxy groups.

In another embodiment, U is N, and R¹ and R² are joined together to form a piperazinyl ring including the N of U as a nitrogen of said piperazinyl ring, which is unsubstituted.

In another embodiment, U is N, and R¹ and R² are joined together to form a piperazinyl ring including the N of U as a nitrogen of said piperazinyl ring, wherein said piperazinyl ring is substituted with 1-3 substituents which can be the same or different, each being independently selected from the group consisting of halo, alkyl, —CN, —NH₂, —NH(alkyl), —N(alkyl)₂, hydroxy and alkoxy groups.

In another embodiment R⁶ is selected from the group consisting of: H, alkyl (e.g. methyl), cycloalkyl (e.g. cyclopropyl), —C(O)OR¹⁵ (e.g. —C(O)OR¹⁵ wherein R¹⁵ is H or alkyl), alkyl substituted with 1-3 halos (e.g. alkyl substituted with 1-3 F), —C(O)R¹⁵ (e.g —C(O)R¹⁵ wherein R¹⁵ is alkyl), and alkyl substituted with —OR¹⁵ (e.g. alkyl substituted with —OR¹⁵ wherein R¹⁵ is H or alkyl, such as, for example, wherein R⁶ is —CH₂OH).

In another embodiment, R⁶ is H.

In another embodiment, R⁶ is alkyl.

In another embodiment, R⁶ is methyl.

In another embodiment R⁶ is alkyl substituted with 1-5 independently selected R²¹ moieties.

In another embodiment R⁶ is alkyl substituted with —OH.

In another embodiment R⁶ is —CH₂OH.

In another embodiment R⁶ is alkyl substituted with 1-5 independently selected R²¹ moieties, and 1 to 3 R²¹ moieties (in one example one, and in another example 2, and in another example 3) are selected from the group consisting of: —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃).

In another embodiment R⁶ is alkyl substituted with 1-5 independently selected R²¹ moieties, wherein 1 to 3 of the R²¹ moieties (in one example one, and in another example 2, and in another example 3) are selected from the group consisting of: —SF₅ and —OSF₅.

In another embodiment R⁶ is alkyl substituted with one R²¹ moiety.

In another embodiment R⁶ is alkyl substituted with one R²¹ moiety, and said R²¹ moiety is selected from the group consisting of: —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃).

In another embodiment R⁶ is alkyl substituted with one R²¹ moiety, and said R²¹ moiety is selected from the group consisting of: —SF₅ and —OSF₅.

In another embodiment R⁶ is alkyl substituted with one R²¹ moiety, and said R²¹ moiety is —OR¹⁵.

In another embodiment R⁶ is alkyl substituted with one R²¹ moiety, said R²¹ moiety is —OR¹⁵, and said R¹⁵ is selected from the group consisting of: H and alkyl.

In another embodiment R⁶ is alkyl substituted with one R²¹ moiety, said R²¹ moiety is —OR¹⁵, and said R¹⁵ is H.

In another embodiment R⁶ is alkyl substituted with one R²¹ moiety, said R²¹ moiety is —OR¹⁵, and said R¹⁵ is alkyl (e.g. methyl).

In another embodiment R⁶ is methyl substituted with 1-3 independently selected R²¹ moieties.

In another embodiment R⁶ is methyl substituted with one R²¹ moiety.

In another embodiment R⁶ is methyl substituted with one R²¹ moiety, and said R²¹ moiety is selected from the group consisting of: —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃).

In another embodiment R⁶ is methyl substituted with one R²¹ moiety, and said R²¹ moiety is selected from the group consisting of: —SF₅ and —OSF₅.

In another embodiment R⁶ is methyl substituted with one R²¹ moiety, and said R²¹ moiety is —OR¹⁵.

In another embodiment R⁶ is methyl substituted with one R²¹ moiety, said R²¹ moiety is —OR¹⁵, and said R¹⁵ is selected from the group consisting of: H and alkyl.

In another embodiment R⁶ is methyl substituted with one R²¹ moiety, said R²¹ moiety is —OR¹⁵, and said R¹⁵ is H.

In another embodiment R⁶ is methyl substituted with one R²¹ moiety, said R²¹ moiety is —OR¹⁵, and said R¹⁵ is alkyl (e.g. methyl).

In another embodiment R⁷ is selected from the group consisting of: (a) aryl substituted with 1-3 R²¹ moieties (e.g. phenyl substituted 1-3 halos, such as, 1-3 F), (b) aryl (e.g. phenyl) substituted with —OR¹⁵ wherein R¹⁵ is (i) an alkyl substituted with 1-3 halos (e.g. F), or (ii) alkyl, (c) aryl (e.g., phenyl), (d) aryl (e.g. phenyl) substituted with alkyl wherein said alkyl is substituted with 1-3 halos (e.g., F), (e) aryl substituted with aryl (e.g. -phenyl-phenyl), (f) alkyl, (g) heteroaryl (e.g. thienyl or pyridyl), (h) arylalkyl-, and (i) cycloalkyl).

In another embodiment R⁷ is aryl (e.g., phenyl) substituted with 1 to 3 independently selected R²¹ moieties wherein at least one R²¹ moiety is selected from the group consisting of —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃).

In another embodiment R⁷ is aryl (e.g., phenyl) substituted with 1 to 3 independently selected R²¹ moieties wherein at least one R²¹ moiety is selected from the group consisting of —SF₅ and —OSF₅.

In another embodiment R⁷ is aryl (e.g., phenyl) substituted with 1 to 3 R²¹ moieties independently selected from the group consisting of: halo (e.g., F), —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃), and wherein at least one R²¹ moiety is selected from the group consisting of —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃).

In another embodiment R⁷ is aryl (e.g., phenyl) substituted with 1 to 3 R²¹ moieties independently selected from the group consisting of: halo (e.g., F), —SF₅ and —OSF₅, and wherein at least one R²¹ moiety is selected from the group consisting of —SF₅ and —OSF₅.

In another embodiment, R⁷ is aryl.

In another embodiment, R⁷ is an unsubstituted phenyl.

In another embodiment, R⁷ is a phenyl which is substituted with 1-4 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment, R⁷ is a phenyl which is substituted with 1-4 substituents independently selected from the group consisting of halo, alkyl, —CN, —NH₂, —NH(alkyl), —N(alkyl)₂, hydroxy, alkoxy, aryl, heteroaryl, —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃), and wherein at least one R²¹ moiety is selected from the group consisting of —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃).

In another embodiment, R⁷ is a phenyl which is substituted with 1-4 substituents independently selected from the group consisting of halo, alkyl, —CN, —NH₂, —NH(alkyl), —N(alkyl)₂, hydroxy, alkoxy, aryl, heteroaryl —SF₅, and —OSF₅, and wherein at least one R²¹ moiety is selected from the group consisting of —SF₅ and —OSF₅.

In another embodiment, R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment, R⁷ is phenyl which is substituted with 1-3 substituents independently selected from the group consisting of halo, alkyl, —CN, —NH₂, —NH(alkyl), —N(alkyl)₂, hydroxy, alkoxy, aryl, heteroaryl, —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃), and wherein at least one R²¹ moiety is selected from the group consisting of —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃).

In another embodiment, R⁷ is phenyl which is substituted with 1-3 substituents independently selected from the group consisting of halo, alkyl, —CN, —NH₂, —NH(alkyl), —N(alkyl)₂, hydroxy, alkoxy, aryl, heteroaryl —SF₅, and —OSF₅, and wherein at least one R²¹ moiety is selected from the group consisting of —SF₅ and —OSF₅.

In another embodiment, R⁷ is phenyl substituted with 1-3 independently selected halos.

In another embodiment, R⁷ is phenyl substituted with 1-3 R²¹ groups independently selected from the group consisting of halos, —SF₅ and —OSF₅, wherein at least one R²¹ group is —SF₅ or —OSF₅.

In another embodiment, R⁷ is phenyl substituted with 1-3 R²¹ groups independently selected from the group consisting of F, Br, —SF₅ and —OSF₅.

In another embodiment, R⁷ is phenyl substituted with 1-3 R²¹ groups independently selected from the group consisting of —SF₅ and —OSF₅.

In another embodiment, R⁷ is phenyl substituted with 1-3 F.

In another embodiment, R⁷ is phenyl substituted with 1-3 R²¹ groups independently selected from the group consisting of F, —SF₅ and —OSF₅, wherein at least one R²¹ group is —SF₅ or —OSF₅.

In another embodiment, R⁷ is phenyl substituted with one —SF₅ group.

In another embodiment, R⁷ is phenyl substituted with two —SF₅ groups.

In another embodiment, R⁷ is phenyl substituted with three —SF₅ groups.

In another embodiment, R⁷ is phenyl substituted with one —OSF₅ group.

In another embodiment, R⁷ is phenyl substituted with two —OSF₅ groups.

In another embodiment, R⁷ is phenyl substituted with three —OSF₅ groups.

In another embodiment, R⁷ is phenyl substituted with 1 F.

In another embodiment, R⁷ is phenyl substituted with 1 F, and also substituted with 1 to 2 groups independently selected from the group consisting of —SF₅ and —OSF₅.

In another embodiment R⁷ is p-Cl-phenyl.

In another embodiment R⁷ is p-Cl-phenyl substituted with 1 to 2 groups independently selected from the group consisting of —SF₅ and —OSF₅.

In another embodiment, R⁷ is unsubstituted naphthyl.

In another embodiment, R⁷ is naphthyl substituted with 1-3 R²¹ groups independently selected from the group consisting of —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃).

In another embodiment, R⁷ is naphthyl substituted with 1-3 R²¹ groups independently selected from the group consisting of —SF₅ and —OSF₅.

In another embodiment, R⁷ is naphthyl substituted with 1-3 R²¹ groups independently selected from the group consisting of halo, —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃), and wherein at least one R²¹ group is selected from the group consisting of —SF₅, —OSF₅ and —Si(R²⁴)₃.

In another embodiment, R⁷ is naphthyl substituted with 1-3 R²¹ groups independently selected from the group consisting of halo, —SF₅ and —OSF₅, wherein at least one R²¹ group is selected from the group consisting of —SF₅ and —OSF₅.

In another embodiment, R⁷ is naphthyl substituted with 1-3 R²¹ groups independently selected from the group consisting of F, Br, —SF₅ and —OSF₅, wherein at least one R²¹ group is selected from the group consisting of —SF₅ and —OSF₅.

In another embodiment, R⁷ is naphthyl substituted with 1-3 R²¹ groups independently selected from the group consisting of F, —SF₅ and —OSF₅, wherein at least one R²¹ group is selected from the group consisting of —SF₅ and —OSF₅.

In another embodiment, R⁷ is naphthyl which is substituted with 1-4 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment, R⁷ is naphthyl substituted with 1-4 R²¹ substituents independently selected from the group consisting of halo, alkyl, —CN, —NH₂, —NH(alkyl), —N(alkyl)₂, hydroxy, alkoxy, aryl, heteroaryl, —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃), and wherein at least one R²¹ group is selected from the group consisting of —SF₅, —OSF₅ and —Si(R²⁴)₃.

In another embodiment, R⁷ is naphthyl substituted with 1-4 R²¹ substituents independently selected from the group consisting of halo, alkyl, —CN, —NH₂, —NH(alkyl), —N(alkyl)₂, hydroxy, alkoxy, aryl, heteroaryl, —SF₅ and —OSF₅, wherein at least one group is —SF₅ or —OSF₅.

In another embodiment, R⁷ is unsubstituted biphenyl.

In another embodiment, R⁷ is biphenyl substituted with 1-3 R²¹ groups independently selected from the group consisting of —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃).

In another embodiment, R⁷ is biphenyl substituted with 1-3 R²¹ groups independently selected from the group consisting of —SF₅ and —OSF₅.

In another embodiment, R⁷ is biphenyl substituted with 1-3 R²¹ groups independently selected from the group consisting of halo, —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃), and wherein at least one R²¹ group is selected from the group consisting of —SF₅, —OSF₅ and —Si(R²⁴)₃.

In another embodiment, R⁷ is biphenyl substituted with 1-3 R²¹ groups independently selected from the group consisting of halo, —SF₅ and —OSF₅, wherein at least one R²¹ group is selected from the group consisting of —SF₅ and —OSF₅.

In another embodiment, R⁷ is biphenyl substituted with 1-3 R²¹ groups independently selected from the group consisting of F, Br, —SF₅ and —OSF₅, wherein at least one R²¹ group is selected from the group consisting of —SF₅ and —OSF₅.

In another embodiment, R⁷ is biphenyl substituted with 1-3 R²¹ groups independently selected from the group consisting of F, —SF₅ and —OSF₅, wherein at least one R²¹ group is selected from the group consisting of —SF₅ and —OSF₅.

In another embodiment, R⁷ is biphenyl which is substituted with 1-4 substituents which can be the same or different, each substituent being independently selected from the group consisting of halo, alkyl, —CN, —NH₂, —NH(alkyl), —N(alkyl)₂, hydroxy and alkoxy groups.

In another embodiment, R⁷ is biphenyl which is substituted with 1-4 R²¹ substituents independently selected from the group consisting of halo, alkyl, —CN, —NH₂, —NH(alkyl), —N(alkyl)₂, hydroxyl, alkoxy, —SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃), and wherein at least one R²¹ group is selected from the group consisting of —SF₅, —OSF₅ and —Si(R²⁴)₃.

In another embodiment, R⁷ is biphenyl which is substituted with 1-4 R²¹ substituents independently selected from the group consisting of halo, alkyl, —CN, —NH₂, —NH(alkyl), —N(alkyl)₂, hydroxyl, alkoxy , —SF₅ and —OSF₅, wherein at least one R²¹ group is selected from the group consisting of —SF₅ and —OSF₅.

In another embodiment, R⁷ is R⁷ is 3-(1,1′-biphenyl)-yl.

In another embodiment, R⁷ is 3-(1,1-biphenyl)-yl substituted with 1-3 R²¹ groups independently selected from the group consisting of —SF₅ and —OSF₅.

In another embodiment, R⁷ is 3-(1,1′-biphenyl)-yl substituted with 1-3 R²¹ groups independently selected from the group consisting of halo, —SF₅ and —OSF₅, wherein at least one R²¹ group is selected from the group consisting of —SF₅ and —OSF₅.

In another embodiment, R⁷ is 3-(1,1′-biphenyl)-yl substituted with 1-3 R²¹ groups independently selected from the group consisting of F, Br, —SF₅ and —OSF₅, wherein at least one R²¹ group is selected from the group consisting of —SF₅ and —OSF₅.

In another embodiment, R⁷ is 3-(1,1′-biphenyl)-yl substituted with 1-3 R²¹ groups independently selected from the group consisting of F, —SF₅ and —OSF₅, wherein at least one R²¹ group is selected from the group consisting of —SF₅ and —OSF₅.

In another embodiment, R⁷ is R⁷ is 4-(1,1′-biphenyl)-yl.

In another embodiment, R⁷ is 4-(1,1′-biphenyl)-yl substituted with 1-3 R²¹ groups independently selected from the group consisting of —SF₅ and —OSF₅.

In another embodiment, R⁷ is 4-(1,1′-biphenyl)-yl substituted with 1-3 R²¹ groups independently selected from the group consisting of halo, —SF₅ and —OSF₅, wherein at least one R²¹ group is selected from the group consisting of —SF₅ and —OSF₅.

In another embodiment, R⁷ is 4-(1,1′-biphenyl)-yl substituted with 1-3 R²¹ groups independently selected from the group consisting of F, Br, —SF₅ and —OSF₅, wherein at least one R²¹ group is selected from the group consisting of —SF₅ and —OSF₅.

In another embodiment, R⁷ is 4-(1,1′-biphenyl)-yl substituted with 1-3 R²¹ groups independently selected from the group consisting of F, —SF₅ and —OSF₅, wherein at least one R²¹ group is selected from the group consisting of —SF₅ and —OSF₅.

In another embodiment, R⁶ is H and R⁷ is a biphenyl which can be unsubstituted or optionally independently substituted with 1-4 substituents which can be the same or different, each substituent being independently selected from the group consisting of halo, alkyl, —CN, —NH₂, —NH(alkyl), —N(alkyl)₂, hydroxy and alkoxy groups.

In another embodiment, R⁶ is H and R⁷ is a biphenyl optionally substituted with 1-4 R²¹ substituents independently selected from the group consisting of halo, alkyl, —CN, —NH₂, —NH(alkyl), —N(alkyl)₂, hydroxyl, alkoxy, —SF₅ and —OSF₅, wherein at least one R²¹ group is selected from the group consisting of —SF₅ and —OSF₅.

In another embodiment, R⁶ is methyl, and R⁷ is a biphenyl which can be unsubstituted or optionally independently substituted with 1-4 substituents which can be the same or different, each substituent being independently selected from the group consisting of halo, alkyl, —CN, —NH₂, —NH(alkyl), —N(alkyl)₂, hydroxy and alkoxy groups.

In another embodiment, R⁶ is methyl, and R⁷ is a biphenyl optionally substituted with 1-4 R²¹ substituents independently selected from the group consisting of halo, alkyl, —CN, —NH₂, —NH(alkyl), —N(alkyl)₂, hydroxyl, alkoxy, —SF₅ and —OSF₅, wherein at least one R²¹ group is selected from the group consisting of —SF₅ and —OSF₅.

In another embodiment, R⁶ is H, and R⁷ is a phenyl which can be unsubstituted or optionally independently substituted with 1-4 substituents which can be the same or different, each substituent being independently selected from the group consisting of halo, alkyl, —CN, —NH₂, —NH(alkyl), —N(alkyl)₂, hydroxy and alkoxy groups.

In another embodiment, R⁶ is H, and R⁷ is a phenyl optionally substituted with 1-4 R²¹ substituents independently selected from the group consisting of halo, alkyl, —CN, —NH₂, —NH(alkyl), —N(alkyl)₂, hydroxyl, alkoxy, —SF₅ and —OSF₅, wherein at least one R²¹ group is selected from the group consisting of —SF₅ and —OSF₅.

In another embodiment, R⁶ and R⁷ are joined to form a spirocyclic unit shown below:

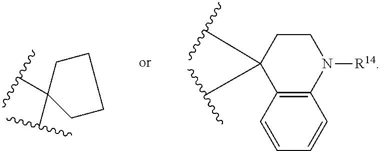

In another embodiment, R⁶ and R⁷ are joined to form the spirocyclic unit

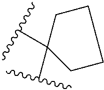

which is substituted with 1 to 3 (e.g. 1 to 3, or 1 to 2, or 1) R²¹ groups independently selected form the group consisting of: —SF₅ and —OSF₅.

In another embodiment, R⁶ and R⁷ are joined to form the spirocyclic unit

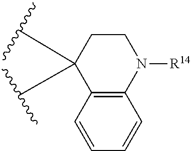

which is substituted with 1 to 3 (e.g. 1 to 3, or 1 to 2, or 1) R²¹ groups independently selected form the group consisting of: —SF₅ and —OSF₅.

In another embodiment, R⁶ and R⁷ are joined to form a spirocyclic unit shown below:

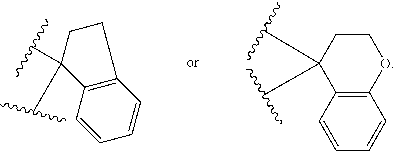

In another embodiment, R⁶ and R⁷ are joined to form the spirocyclic unit

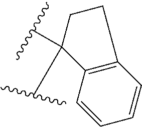

which is substituted with 1 to 3 (e.g. 1 to 3, or 1 to 2, or 1) R²¹ groups independently selected form the group consisting of: —SF₅ and —OSF₅.

In another embodiment, R⁶ and R⁷ are joined to form the spirocyclic unit

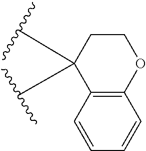

which is substituted with 1 to 3 (e.g. 1 to 3, or 1 to 2, or 1) R²¹ groups independently selected form the group consisting of: —SF₅ and —OSF₅.

In another embodiment R⁶ is alkyl, and R⁷ is phenyl substituted with 1-3 R²¹ substituents independently selected from the group consisting of halo, alkyl, —CN, —NH₂, —NH(alkyl), —N(alkyl)₂, hydroxy, alkoxy, aryl, heteroaryl, halo, alkyl, —CN, —NH₂, —NH(alkyl), —N(alkyl)₂, hydroxyl, alkoxy, SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃), and wherein at least one R²¹ group is selected from the group consisting of —SF₅, —OSF₅ and —Si(R²⁴)₃.

In another embodiment R⁶ is alkyl, and R⁷ is phenyl substituted with 1-3 R²¹ substituents independently selected from the group consisting of halo, alkyl, —CN, —NH₂, —NH(alkyl), —N(alkyl)₂, hydroxy, alkoxy, aryl, heteroaryl, halo, alkyl, —CN, —NH₂, —NH(alkyl), —N(alkyl)₂, hydroxyl, alkoxy, —SF₅ and —OSF₅, wherein at least one R²¹ group on said phenyl is selected from the group consisting of —SF₅ and —OSF₅.

In another embodiment R⁶ is alkyl substituted with 1-5 independently selected R²¹ moieties, and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment R⁶ is alkyl substituted with 1-5 independently selected R²¹ moieties, and R⁷ is phenyl substituted with 1-3 R²¹ substituents independently selected from the group consisting of halo, alkyl, —CN, —NH₂, —NH(alkyl), —N(alkyl)₂, hydroxy, alkoxy, aryl, heteroaryl, halo, alkyl, —CN, —NH₂, —NH(alkyl), —N(alkyl)₂, hydroxyl, alkoxy, SF₅, —OSF₅ and —Si(R²⁴)₃ (and in one example each R²⁴ is the same or different alkyl, and in another example the —Si(R²⁴)₃ group is —Si(CH₃)₃ or —Si(CH₂CH₃)₂CH_{3,} and in another example the —Si(R²⁴)₃ group is —Si(CH₃)₃), and wherein at least one R²¹ group is selected from the group consisting of —SF₅, —OSF₅ and —Si(R²⁴)₃.

In another embodiment R⁶ is alkyl substituted with 1-5 independently selected R²¹ moieties, and R⁷ is phenyl substituted with 1-3 R²¹ substituents independently selected from the group consisting of halo, alkyl, —CN, —NH₂, —NH(alkyl), —N(alkyl)₂, hydroxy, alkoxy, aryl, heteroaryl, halo, alkyl, —CN, —NH₂, —NH(alkyl), —N(alkyl)₂, hydroxyl, alkoxy, —SF₅ and —OSF₅, wherein at least one R²¹ group on said phenyl is selected from the group consisting of —SF₅ and —OSF₅.

In another embodiment R⁶ is alkyl substituted with one R²¹ moiety, and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment R⁶ is alkyl substituted with one R²¹ moiety, and R⁷ is phenyl substituted with 1-3 R²¹ substituents independently selected from the group consisting of halo, alkyl, —CN, —NH₂, —NH(alkyl), —N(alkyl)₂, hydroxy, alkoxy, aryl, heteroaryl, SF₅ and —OSF₅, wherein at least one R²¹ group on said phenyl is selected from the group consisting of —SF₅ and —OSF₅.

In another embodiment R⁶ is alkyl substituted with one R²¹ moiety, and R⁷ is phenyl substituted with 1-3 independently selected halos.

In another embodiment R⁶ is alkyl substituted with one R²¹ moiety, and R⁷ is phenyl substituted with 1-3 R²¹ groups independently selected from the group consisting of halos, SF₅ and —OSF₅, wherein at least one R²¹ group on said phenyl is selected from the group consisting of —SF₅ and —OSF₅.

In another embodiment R⁶ is alkyl substituted with one R²¹ moiety, and R⁷ is phenyl substituted with 1-3 F.

In another embodiment R⁶ is alkyl substituted with one R²¹ moiety, and R⁷ is phenyl substituted with 1-3 R²¹ groups independently selected from the group consisting of F, SF₅ and —OSF₅, wherein at least one R²¹ group on said phenyl is selected from the group consisting of —SF₅ and —OSF₅.

In another embodiment R⁶ is alkyl substituted with one R²¹ moiety, and R⁷ is phenyl substituted with one F.

In another embodiment R⁶ is alkyl substituted with one R²¹ moiety, and R⁷ is phenyl substituted with one F, and one or two groups independently selected from the group consisting of SF₅ and —OSF₅, wherein at least one —SF₅ or —OSF₅ is present.

In another embodiment R⁶ is alkyl substituted with one R²¹ moiety, and R⁷ is phenyl substituted with one F, and one or two groups independently selected from the group consisting of SF₅ and —OSF₅, wherein at least one —SF₅ or —OSF₅ is present.

In another embodiment R⁶ is alkyl substituted with one R²¹ moiety, and R⁷ is phenyl substituted with 1 to 3 groups independently selected from the group consisting of SF₅ and —OSF₅.

In another embodiment R⁶ is alkyl substituted with one R²¹ moiety, and R⁷ is phenyl substituted with 1 to 2 groups independently selected from the group consisting of SF₅ and —OSF₅.

In another embodiment R⁶ is alkyl substituted with one R²¹ moiety, and R⁷ is phenyl substituted with 1 to 3 —SF₅ groups (and in one example one —SF₅, and in another example two —SF₅ groups, and in another example three —SF₅ groups).

In another embodiment R⁶ is alkyl substituted with one R²¹ moiety, and R⁷ is phenyl substituted with 1 to 3 —OSF₅ groups (and in one example one —OSF₅, and in another example two —OSF₅ groups, and in another example three —OSF₅ groups).

In another embodiment R⁶ is alkyl substituted with one R²¹ moiety, and said R²¹ moiety is —OR¹⁵, and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment R⁶ is alkyl substituted with one R²¹ moiety, and said R²¹ moiety is —OR¹⁵, and R⁷ is phenyl substituted with 1-3 R²¹ independently selected from the group consisting of halo, alkyl, —CN, —NH₂, —NH(alkyl), —N(alkyl)₂, hydroxy, alkoxy, aryl, heteroaryl, —SF₅ and —OSF₅, wherein at least one R²¹ group on said phenyl is selected from the group consisting of —SF₅ and —OSF₅.

In another embodiment R⁶ is alkyl substituted with one R²¹ moiety, and said R²¹ moiety is —OR¹⁵, and R⁷ is phenyl substituted with 1-3 independently selected halos.

In another embodiment R⁶ is alkyl substituted with one R²¹ moiety, and said R²¹ moiety is —OR¹⁵, and R⁷ is phenyl substituted with 1-3 R²¹ groups independently selected from the group consisting of halos, —SF₅ and —OSF₅, wherein at least one R²¹ group on said phenyl is selected from the group consisting of —SF₅ and —OSF₅.

In another embodiment R⁶ is alkyl substituted with one R²¹ moiety, and said R²¹ moiety is —OR¹⁵, and R⁷ is phenyl substituted with 1-3 independently selected F.

In another embodiment R⁶ is alkyl substituted with one R²¹ moiety, and said R²¹ moiety is —OR¹⁵, and R⁷ is phenyl substituted with 1-3 R²¹ groups independently selected from the group consisting of F, —SF₅ and —OSF₅, wherein at least one R²¹ group on said phenyl is selected from the group consisting of —SF₅ and —OSF₅.

In another embodiment R⁶ is alkyl substituted with one R²¹ moiety, and said R²¹ moiety is —OR¹⁵, and R⁷ is phenyl substituted with one F.

In another embodiment R⁶ is alkyl substituted with one R²¹ moiety, and said R²¹ moiety is —OR¹⁵, and R⁷ is phenyl substituted with one F, and and said phenyl is also subsubstitued with one or two groups independently selected from the group consisting of: —SF₅ and —OSF₅, wherein at least one R²¹ group on said phenyl is selected from the group consisting of —SF₅ and —OSF₅.

In another embodiment R⁶ is alkyl substituted with one R²¹ moiety, said R²¹ moiety is —OR¹⁵, and said R¹⁵ is selected from the group consisting of: H and alkyl, and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment R⁶ is alkyl substituted with one R²¹ moiety, said R²¹ moiety is —OR¹⁵, and said R¹⁵ is selected from the group consisting of: H and alkyl, and R⁷ is phenyl substituted with 1-3 R²¹ substituents independently selected from the group consisting of halo, alkyl, —CN, —NH₂, —NH(alkyl), —N(alkyl)₂, hydroxy, alkoxy, aryl, heteroaryl, —SF₅ and —OSF₅, wherein at least one R²¹ group on said phenyl is selected from the group consisting of —SF₅ and —OSF₅.

In another embodiment R⁶ is alkyl substituted with one R²¹ moiety, said R²¹ moiety is —OR¹⁵, and said R¹⁵ is selected from the group consisting of: H and alkyl, and R⁷ is phenyl substituted with 1-3 independently selected halos.

In another embodiment R⁶ is alkyl substituted with one R²¹ moiety, said R²¹ moiety is —OR¹⁵, and said R¹⁵ is selected from the group consisting of: H and alkyl, and R⁷ is phenyl substituted with 1-3 R²¹ groups independently selected from the group consisting of halos, —SF₅ and —OSF₅, wherein at least one R²¹ group on said phenyl is selected from the group consisting of —SF₅ and —OSF₅.

In another embodiment R⁶ is alkyl substituted with one R²¹ moiety, said R²¹ moiety is —OR¹⁵, and said R¹⁵ is selected from the group consisting of: H and alkyl, and R⁷ is phenyl substituted with 1-3 independently selected F.

In another embodiment R⁶ is alkyl substituted with one R²¹ moiety, said R²¹ moiety is —OR¹⁵, and said R¹⁵ is selected from the group consisting of: H and alkyl, and R⁷ is phenyl substituted with 1-3 R²¹ groups independently selected from the group consisting of F, —SF₅ and —OSF₅, wherein at least one R²¹ group on said phenyl is selected from the group consisting of —SF₅ and —OSF₅.

In another embodiment R⁶ is alkyl substituted with one R²¹ moiety, said R²¹ moiety is —OR¹⁵, and said R¹⁵ is selected from the group consisting of: H and alkyl, and R⁷ is phenyl substituted with one F.

In another embodiment R⁶ is alkyl substituted with one R²¹ moiety, said R²¹ moiety is —OR¹⁵, and said R¹⁵ is selected from the group consisting of: H and alkyl, and R⁷ is phenyl substituted with one F, and also substituted with one or two groups independently selected from the group consisting of —SF₅ and —OSF₅.

In another embodiment R⁶ is alkyl substituted with one R²¹ moiety, said R²¹ moiety is —OR¹⁵, and said R¹⁵ is H, and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment R⁶ is alkyl substituted with one R²¹ moiety, said R²¹ moiety is —OR¹⁵, and said R¹⁵ is H, and R⁷ is phenyl substituted with 1-3 R²¹ groups independently selected from the group consisting of halo, alkyl, —CN, —NH₂, —NH(alkyl), —N(alkyl)₂, hydroxy, alkoxy, aryl, heteroaryl, —SF₅ and —OSF₅, wherein at least one R²¹ group on said phenyl is selected from the group consisting of —SF₅ and —OSF₅.

In another embodiment R⁶ is alkyl substituted with one R²¹ moiety, said R²¹ moiety is —OR¹⁵, and said R¹⁵ is H, and said R¹⁵ is selected from the group consisting of: H and alkyl, and R⁷ is phenyl substituted with 1-3 independently selected halos.

In another embodiment R⁶ is alkyl substituted with one R²¹ moiety, said R²¹ moiety is —OR¹⁵, and said R¹⁵ is H, and said R¹⁵ is selected from the group consisting of: H and alkyl, and R⁷ is phenyl substituted with 1-3 R²¹ groups independently selected from the group consisting of halos, —SF₅ and —OSF₅, wherein at least one R²¹ group on said phenyl is selected from the group consisting of —SF₅ and —OSF₅.

In another embodiment R⁶ is alkyl substituted with one R²¹ moiety, said R²¹ moiety is —OR¹⁵, and said R¹⁵ is H, and said R¹⁵ is selected from the group consisting of: H and alkyl, and R⁷ is phenyl substituted with 1-3 independently selected F.

In another embodiment R⁶ is alkyl substituted with one R²¹ moiety, said R²¹ moiety is —OR¹⁵, and said R¹⁵ is H, and said R¹⁵ is selected from the group consisting of: H and alkyl, and R⁷ is phenyl substituted with 1-2 F, and said phenyl is also substituted with 1 to 2 R²¹ groups independently selected from the group consisting of —SF₅ and —OSF₅, wherein at least one R²¹ group on said phenyl is selected from the group consisting of —SF₅ and —OSF₅, and wherein the total number of substituents on said phenyl is 2 to 3.

In another embodiment R⁶ is alkyl substituted with one R²¹ moiety, said R²¹ moiety is —OR¹⁵, and said R¹⁵ is H, and said R¹⁵ is selected from the group consisting of: H and alkyl, and R⁷ is phenyl substituted with one F.

In another embodiment R⁶ is alkyl substituted with one R²¹ moiety, said R²¹ moiety is —OR¹⁵, and said R¹⁵ is H, and said R¹⁵ is selected from the group consisting of: H and alkyl, and R⁷ is phenyl substituted with one F, and said phenyl is also substituted with one or two groups selected from the group consisting of —SF₅ and —OSF₅, wherein at least one R²¹ group on said phenyl is selected from the group consisting of —SF₅ and —OSF₅.

In another embodiment R⁶ is alkyl substituted with one R²¹ moiety, said R²¹ moiety is —OR¹⁵, and said R¹⁵ is alkyl (e.g. methyl), and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment R⁶ is alkyl substituted with one R²¹ moiety, said R²¹ moiety is —OR¹⁵, and said R¹⁵ is alkyl (e.g. methyl), and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl, heteroaryl, —SF₅ and —OSF₅, wherein at least one R²¹ group on said phenyl is selected from the group consisting of —SF₅ and —OSF₅.

In another embodiment R⁶ is alkyl substituted with one R²¹ moiety, said R²¹ moiety is —OR¹⁵, and said R¹⁵ is alkyl (e.g. methyl), and R⁷ is phenyl substituted with 1-3 independently selected halos.

In another embodiment R⁶ is alkyl substituted with one R²¹ moiety, said R²¹ moiety is —OR¹⁵, and said R¹⁵ is alkyl (e.g. methyl), and R⁷ is phenyl substituted with 1-3 R²¹ groups independently selected from the group consisting of halos, —SF₅ and —OSF₅, wherein at least one R²¹ group on said phenyl is selected from the group consisting of —SF₅ and —OSF₅.

In another embodiment R⁶ is alkyl substituted with one R²¹ moiety, said R²¹ moiety is —OR¹⁵, and said R¹⁵ is alkyl (e.g. methyl), and R⁷ is phenyl substituted with 1-3 independently selected F.

In another embodiment R⁶ is alkyl substituted with one R²¹ moiety, said R²¹ moiety is —OR¹⁵, and said R¹⁵ is alkyl (e.g. methyl), and R⁷ is phenyl substituted with 1-2 F, and said phenyl is also substituted with one or two groups independently selected from the group consisting of —SF₅ and —OSF₅, and wherein the total number of substituents on said phenyl is 2 to 3.

In another embodiment R⁶ is alkyl substituted with one R²¹ moiety, said R²¹ moiety is —OR¹⁵, and said R¹⁵ is alkyl (e.g. methyl), and R⁷ is phenyl substituted with one F.

In another embodiment R⁶ is alkyl substituted with one R²¹ moiety, said R²¹ moiety is —OR¹⁵, and said R¹⁵ is alkyl (e.g. methyl), and R⁷ is phenyl substituted with one F, and said phenyl is also substituted with one or two substituents selected from the group consisting of —SF₅ and —OSF₅.

In another embodiment R⁶ is methyl substituted with 1-3 independently selected R²¹ moieties, and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment R⁶ is methyl substituted with 1-3 independently selected R²¹ moieties, and R⁷ is phenyl substituted with 1-3 R²¹ substituents independently selected from the group consisting of halo, alkyl, —CN, —NH₂, —NH(alkyl), —N(alkyl)₂, hydroxy, alkoxy, aryl, heteroaryl, —SF₅ and —OSF₅, wherein at least one R²¹ group on said phenyl is selected from the group consisting of —SF₅ and —OSF₅.

In another embodiment R⁶ is methyl substituted with one R²¹ moiety, and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment R⁶ is methyl substituted with one R²¹ moiety, and R⁷ is phenyl substituted with 1-3 R²¹ independently selected from the group consisting of halo, alkyl, —CN, —NH₂, —NH(alkyl), —N(alkyl)₂, hydroxy, alkoxy, aryl, heteroaryl, —SF₅ and —OSF₅, wherein at least one R²¹ group on said phenyl is selected from the group consisting of —SF₅ and —OSF₅.

In another embodiment R⁶ is methyl substituted with one R²¹ moiety, and R⁷ is phenyl substituted with 1-3 independently selected halos.

In another embodiment R⁶ is methyl substituted with one R²¹ moiety, and R⁷ is phenyl substituted with 1-2 independently selected halos, and said phenyl is also substituted with one or two groups independently selected from the group consisting of —SF₅ and —OSF₅, and wherein the total number of substituents on said phenyl is 2 or three.

In another embodiment R⁶ is methyl substituted with one R²¹ moiety, and R⁷ is phenyl substituted with 1-3 F.

In another embodiment R⁶ is methyl substituted with one R²¹ moiety, and R⁷ is phenyl substituted with 1-2 F, and said phenyl is also substituted with one or two groups independently selected from the group consisting of —SF₅ and —OSF₅, and wherein the total number of substituents on said phenyl is 2 or three.

In another embodiment R⁶ is methyl substituted with one R²¹ moiety, and R⁷ is phenyl substituted with one F.

In another embodiment R⁶ is methyl substituted with one R²¹ moiety, and R⁷ is phenyl substituted with one F, and said phenyl is also substituted with one or two groups independently selected from the group consisting of —SF₅ and —OSF₅.

In another embodiment R⁶ is methyl substituted with one R²¹ moiety, and said R²¹ moiety is —OR¹⁵, and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment R⁶ is methyl substituted with one R²¹ moiety, and said R²¹ moiety is —OR¹⁵, and R⁷ is phenyl substituted with 1-3 substituents independently selected from the group consisting of halo, alkyl, —CN, —NH₂, —NH(alkyl), —N(alkyl)₂, hydroxy, alkoxy, aryl, heteroaryl, —SF₅ and —OSF₅, wherein at least one R²¹ group on said phenyl is selected from the group consisting of —SF₅ and —OSF₅.

In another embodiment R⁶ is methyl substituted with one R²¹ moiety, and said R²¹ moiety is —OR¹⁵, and R⁷ is phenyl substituted with 1-3 independently selected halos.

In another embodiment R⁶ is methyl substituted with one R²¹ moiety, and said R²¹ moiety is —OR¹⁵, and R⁷ is phenyl substituted with 1-2 independently selected halos, and said phenyl is also substituted with one or two groups independently selected from the group consisting of —SF₅ and —OSF₅, and wherein the total number of substituents on said phenyl is 2 or 3.

In another embodiment R⁶ is methyl substituted with one R²¹ moiety, and said R²¹ moiety is —OR¹⁵, and R⁷ is phenyl substituted with 1-3 independently selected F.

In another embodiment R⁶ is methyl substituted with one R²¹ moiety, and said R²¹ moiety is —OR¹⁵, and R⁷ is phenyl substituted with 1-2 independently selected F, and said phenyl is also substituted with one or two groups independently selected from the group consisting of —SF₅ and —OSF₅, and wherein the total number of substituents on said phenyl is 2 or 3.

In another embodiment R⁶ is methyl substituted with one R²¹ moiety, and said R²¹ moiety is —OR¹⁵, and R⁷ is phenyl substituted with one F.

In another embodiment R⁶ is methyl substituted with one R²¹ moiety, and said R²¹ moiety is —OR¹⁵, and R⁷ is phenyl substituted with one F, and said phenyl is also substituted with one or two groups independently selected from the group consisting of —SF₅ and —OSF₅.

In another embodiment R⁶ is methyl substituted with one R²¹ moiety, said R²¹ moiety is —OR¹⁵, and said R¹⁵ is selected from the group consisting of: H and alkyl, and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment R⁶ is methyl substituted with one R²¹ moiety, said R²¹ moiety is —OR¹⁵, and said R¹⁵ is selected from the group consisting of: H and alkyl, and R⁷ is phenyl substituted with 1-3 substituents independently selected from the group consisting of halo, alkyl, —CN, —NH₂, —NH(alkyl), —N(alkyl)₂, hydroxy, alkoxy, aryl, heteroaryl, —SF₅ and —OSF₅, wherein at least one R²¹ group on said phenyl is selected from the group consisting of —SF₅ and —OSF₅.

In another embodiment R⁶ is methyl substituted with one R²¹ moiety, said R²¹ moiety is —OR¹⁵, and said R¹⁵ is selected from the group consisting of: H and alkyl, and R⁷ is phenyl substituted with 1-3 independently selected halos.

In another embodiment R⁶ is methyl substituted with one R²¹ moiety, said R²¹ moiety is —OR¹⁵, and said R¹⁵ is selected from the group consisting of: H and alkyl, and R⁷ is phenyl substituted with 1-2 independently selected halos, and said phenyl is also substituted with one or two groups independently selected from the group consisting of —SF₅ and —OSF₅, and wherein the total number of substituents on said phenyl is 2 or 3.

In another embodiment R⁶ is methyl substituted with one R²¹ moiety, said R²¹ moiety is —OR¹⁵, and said R¹⁵ is selected from the group consisting of: H and alkyl, and R⁷ is phenyl substituted with 1-3 independently selected F.

In another embodiment R⁶ is methyl substituted with one R²¹ moiety, said R²¹ moiety is —OR¹⁵, and said R¹⁵ is selected from the group consisting of: H and alkyl, and R⁷ is phenyl substituted with 1-2 independently selected F, and said phenyl is also substituted with one or two groups independently selected from the group consisting of —SF₅ and —OSF₅, and wherein the total number of substituents on said phenyl is 2 or 3.

In another embodiment R⁶ is methyl substituted with one R²¹ moiety, said R²¹ moiety is —OR¹⁵, and said R¹⁵ is selected from the group consisting of: H and alkyl, and R⁷ is phenyl substituted with one F.

In another embodiment R⁶ is methyl substituted with one R²¹ moiety, said R²¹ moiety is —OR¹⁵, and said R¹⁵ is selected from the group consisting of: H and alkyl, and R⁷ is phenyl substituted with one F, and said phenyl is also substituted with one or two groups independently selected from the group consisting of —SF₅ and —OSF₅.

In another embodiment R⁶ is methyl substituted with one R²¹ moiety, said R²¹ moiety is —OR¹⁵, and said R¹⁵ is H, and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment R⁶ is methyl substituted with one R²¹ moiety, said R²¹ moiety is —OR¹⁵, and said R¹⁵ is H, and R⁷ is phenyl substituted with 1-3 substituents independently selected from the group consisting of halo, alkyl, —CN, —NH₂, —NH(alkyl), —N(alkyl)₂, hydroxy, alkoxy, aryl, heteroaryl, —SF₅ and —OSF₅, wherein at least one R²¹ group on said phenyl is selected from the group consisting of —SF₅ and —OSF₅.

In another embodiment R⁶ is methyl substituted with one R²¹ moiety, said R²¹ moiety is —OR¹⁵, and said R¹⁵ is H, and said R¹⁵ is selected from the group consisting of: H and alkyl, and R⁷ is phenyl substituted with 1-3 independently selected halos.

In another embodiment R⁶ is methyl substituted with one R²¹ moiety, said R²¹ moiety is —OR¹⁵, and said R¹⁵ is H, and said R¹⁵ is selected from the group consisting of: H and alkyl, and R⁷ is phenyl substituted with 1-2 independently selected halos, and said phenyl is also substituted with one or two groups independently selected from the group consisting of —SF₅ and —OSF₅, and wherein the total number of substituents on said phenyl is 2 or 3.

In another embodiment R⁶ is methyl substituted with one R²¹ moiety, said R²¹ moiety is —OR¹⁵, and said R¹⁵ is H, and said R¹⁵ is selected from the group consisting of: H and alkyl, and R⁷ is phenyl substituted with 1-3 F.

In another embodiment R⁶ is methyl substituted with one R²¹ moiety, said R²¹ moiety is —OR¹⁵, and said R¹⁵ is H, and said R¹⁵ is selected from the group consisting of: H and alkyl, and R⁷ is phenyl substituted with 1-2 F, and said phenyl is also substituted with one or two groups independently selected from the group consisting of —SF₅ and —OSF₅, and wherein the total number of substituents on said phenyl is 2 or 3.

In another embodiment R⁶ is methyl substituted with one R²¹ moiety, said R²¹ moiety is —OR¹⁵, and said R¹⁵ is H, and said R¹⁵ is selected from the group consisting of: H and alkyl, and R⁷ is phenyl substituted with one F.

In another embodiment R⁶ is methyl substituted with one R²¹ moiety, said R²¹ moiety is —OR¹⁵, and said R¹⁵ is H, and said R¹⁵ is selected from the group consisting of: H and alkyl, and R⁷ is phenyl substituted with one F, and said phenyl is also substituted with one or two groups independently selected from the group consisting of —SF₅ and —OSF₅.

In another embodiment R⁶ is methyl substituted with one R²¹ moiety, said R²¹ moiety is —OR¹⁵, and said R¹⁵ is alkyl (e.g. methyl), and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups.

In another embodiment R⁶ is methyl substituted with one R²¹ moiety, said R²¹ moiety is —OR¹⁵, and said R¹⁵ is alkyl (e.g. methyl), and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl, heteroaryl, —SF₅ and —OSF₅, wherein at least one R²¹ group on said phenyl is selected from the group consisting of —SF₅ and —OSF₅.

In another embodiment R⁶ is methyl substituted with one R²¹ moiety, said R²¹ moiety is —OR¹⁵, and said R¹⁵ is alkyl (e.g. methyl), and R⁷ is phenyl substituted with 1-3 independently selected halos.

In another embodiment R⁶ is methyl substituted with one R²¹ moiety, said R²¹ moiety is —OR¹⁵, and said R¹⁵ is alkyl (e.g. methyl), and R⁷ is phenyl substituted with 1-2 independently selected halos, and said phenyl is also substituted with one or two groups independently selected from the group consisting of —SF₅ and —OSF₅, and wherein the total number of substituents on said phenyl is 2 or 3.

In another embodiment R⁶ is methyl substituted with one R²¹ moiety, said R²¹ moiety is —OR¹⁵, and said R¹⁵ is alkyl (e.g. methyl), and R⁷ is phenyl substituted with 1-3 independently selected F.

In another embodiment R⁶ is methyl substituted with one R²¹ moiety, said R²¹ moiety is —OR¹⁵, and said R¹⁵ is alkyl (e.g. methyl), and R⁷ is phenyl substituted with 1-2 independently selected F, and said phenyl is also substituted with one or two groups independently selected from the group consisting of —SF₅ and —OSF₅, and wherein the total number of substituents on said phenyl is 2 or 3.

In another embodiment R⁶ is methyl substituted with one R²¹ moiety, said R²¹ moiety is —OR¹⁵, and said R¹⁵ is alkyl (e.g. methyl), and R⁷ is phenyl substituted with one F.

In another embodiment R⁶ is methyl substituted with one R²¹ moiety, said R²¹ moiety is —OR¹⁵, and said R¹⁵ is alkyl (e.g. methyl), and R⁷ is phenyl substituted with one F, and said phenyl is also substituted with one or two groups independently selected from the group consisting of —SF₅ and —OSF₅.

In another embodiment R⁶ is selected from the group consisting of: H, methyl, cyclopropyl, —C(O)CH₂CH₃, —CHF₂, —CF₃, —C(O)OCH₃, —CH₂OH, —CH₂OCH₃, —CH₂OCH(CH₃)₂, —C(O)OH, —C(CH₃)₃, —C(OH)(CH₃)₂, —C(O)CH₃, —CH(CH₃)OH, —CH₂C(OH)(CH₃)₂, —CH₂CH₂OH and

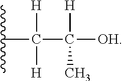

In another embodiment R⁷ is selected from the group consisting of:

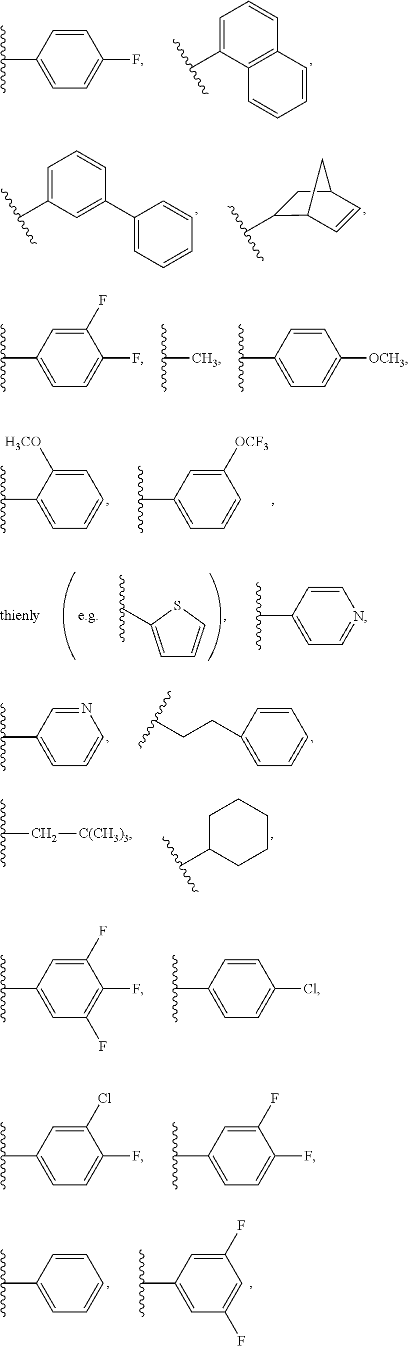

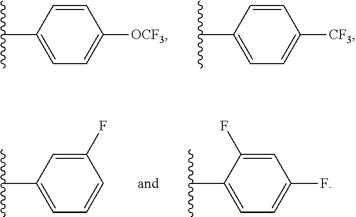

In another embodiment R⁷ is selected from the group consisting of:

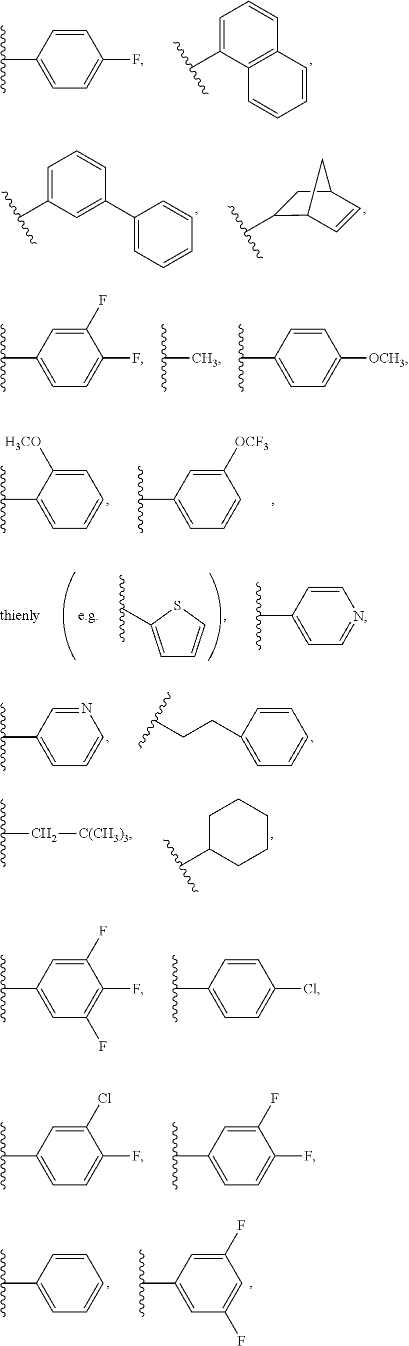

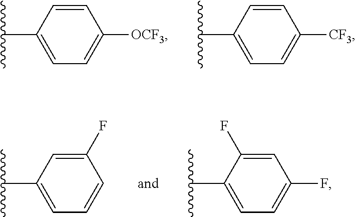

and wherein said R⁷ groups are substituted with 1 to 3 substituents independently selected from the group consisting of: —SF₅, —OSF₅, and —Si(R²⁴)₃ wherein each R²⁴ is the same or different alkyl group.

In another embodiment R⁷ is selected from the group consisting of:

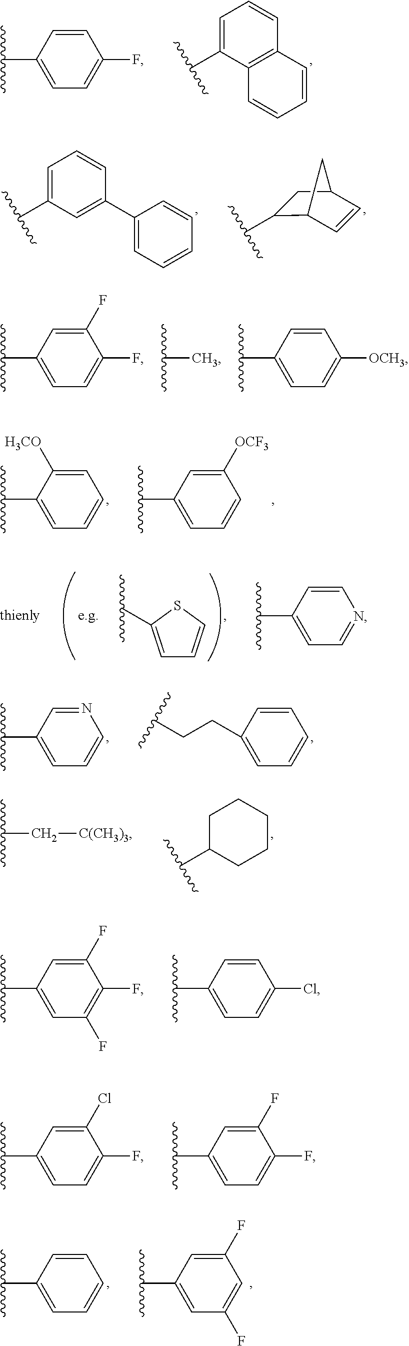

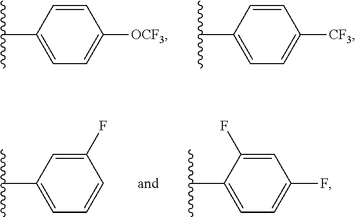

and wherein said R⁷ groups are substituted with 1 to 3 substituents independently selected from the group consisting of: —SF₅ and —OSF₅.

In another embodiment R⁶ and R⁷ taken together with the carbon to which they are bound form a spiro ring selected from the group consisting of:

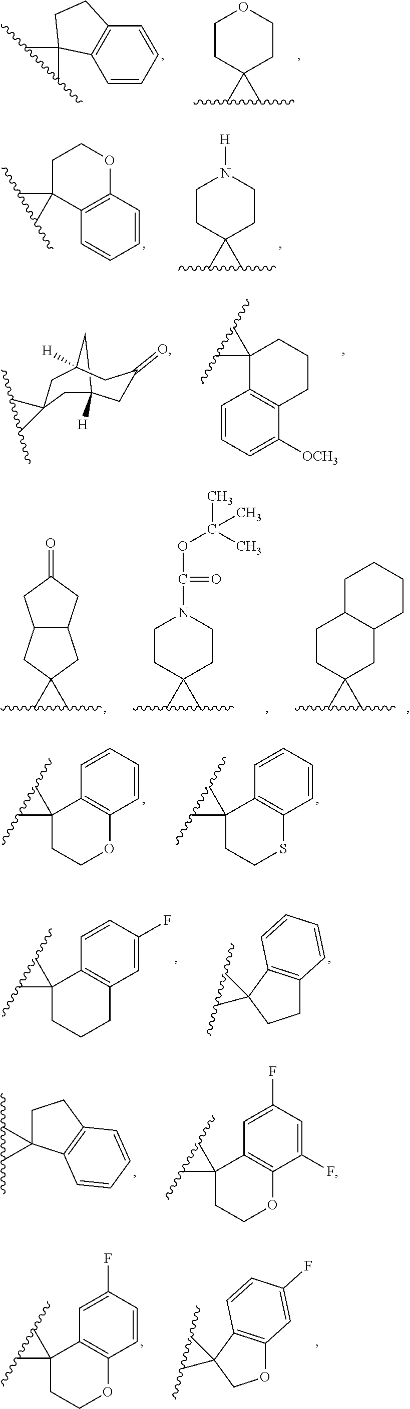

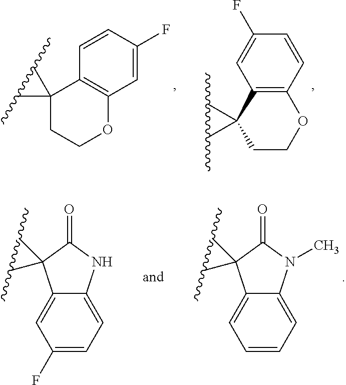

In another embodiment R⁶ and R⁷ taken together with the carbon to which they are bound form a Spiro ring selected from the group consisting of:

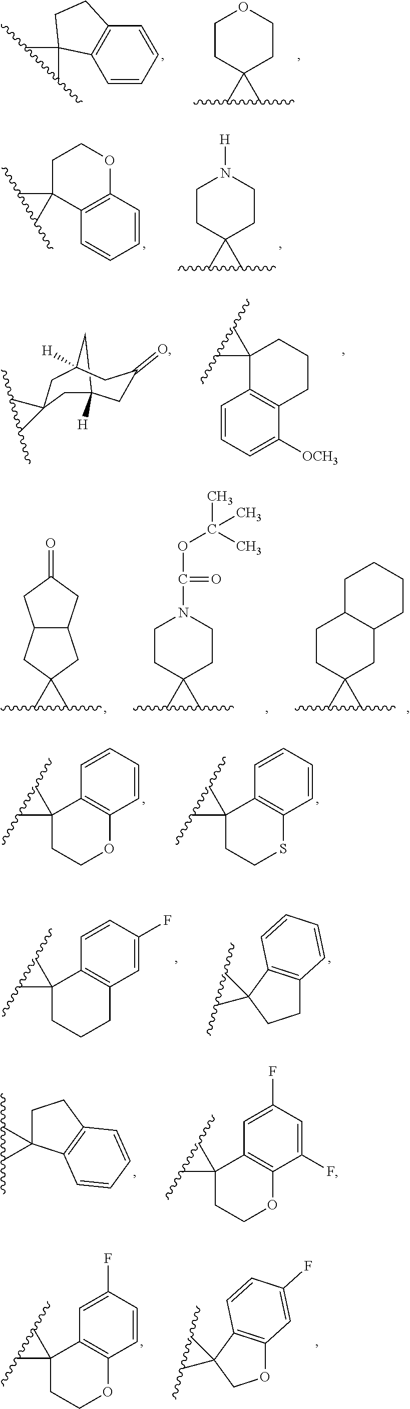

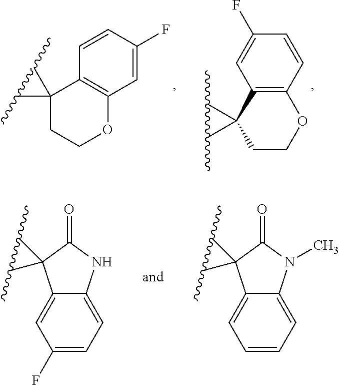

and wherein said spiro rings are substituted with 1 to 3 substituents independently selected from the group consisting of: —SF₅, —OSF₅, and —Si(R²⁴)₃ wherein each R²⁴ is the same or different alkyl group.

In another embodiment R⁶ and R⁷ taken together with the carbon to which they are bound form a Spiro ring selected from the group consisting of:

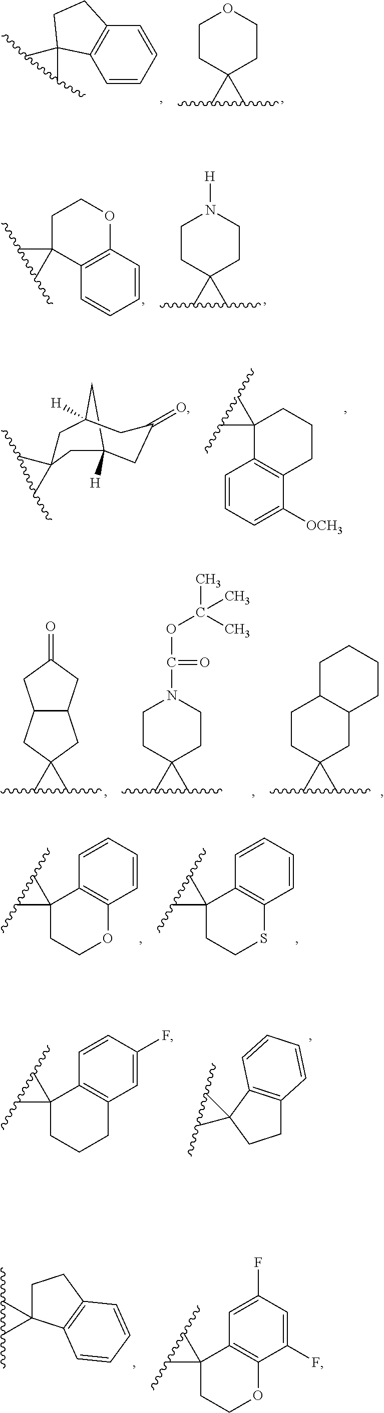

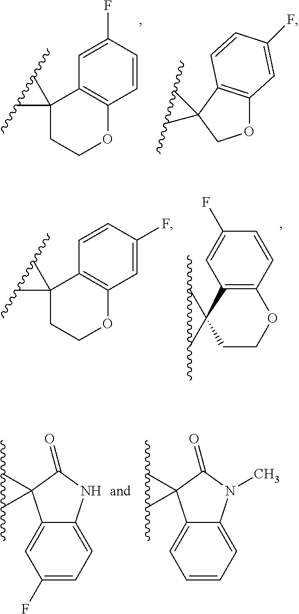

and wherein said Spiro rings are substituted with 1 to 3 substituents independently selected from the group consisting of: —SF₅ and —OSF₅.

In another embodiment R⁶ is selected from the group consisting of: H, methyl, cyclopropyl, —C(O)CH₂CH₃, —CHF₂, —CF₃, —C(O)OCH₃, —CH₂OH, —CH₂OCH₃, —CH₂OCH(CH₃)₂, —C(O)OH, —C(CH₃)₃, —C(OH)(CH₃)₂, —C(O)CH₃, —CH(CH₃)OH, —CH₂C(OH)(CH₃)₂, —CH₂CH₂OH and

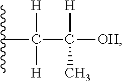

and R⁷ is selected from the group consisting of:

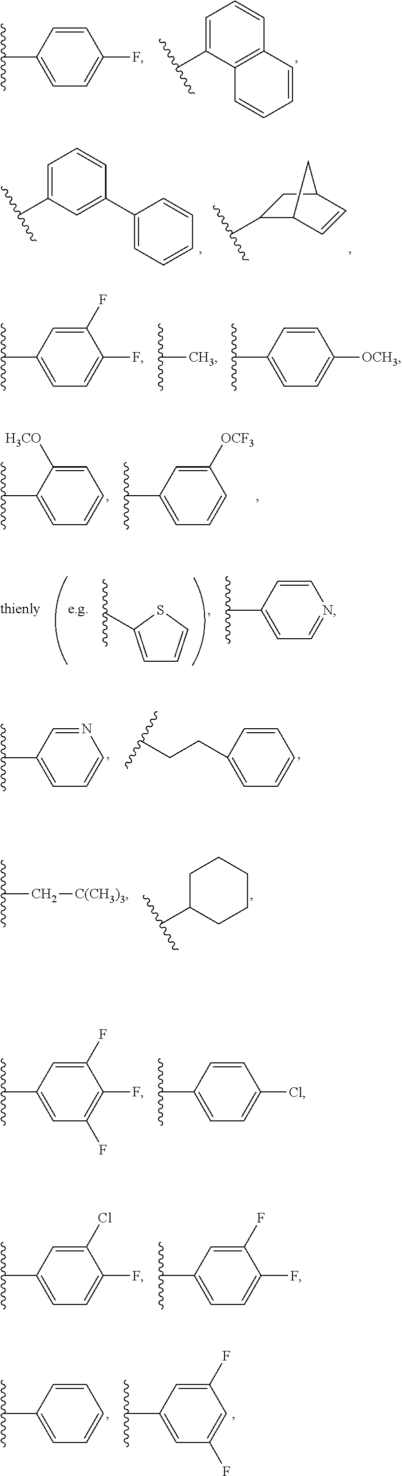

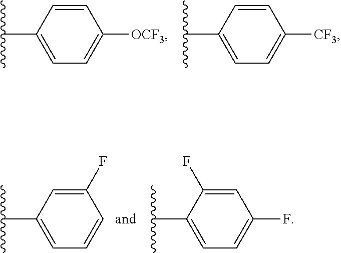

In another embodiment R⁶ is selected from the group consisting of: H, methyl, cyclopropyl, —C(O)CH₂CH₃, —CHF₂, —CF₃, —C(O)OCH₃, —CH₂OH, —CH₂OCH₃, —CH₂OCH(CH₃)₂, —C(O)OH, —C(CH₃)₃, —C(OH)(CH₃)₂, —C(O)CH₃, —CH(CH₃)OH, —CH₂C(OH)(CH₃)₂, —CH₂CH₂OH and

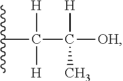

and R⁷ is selected from the group consisting of:

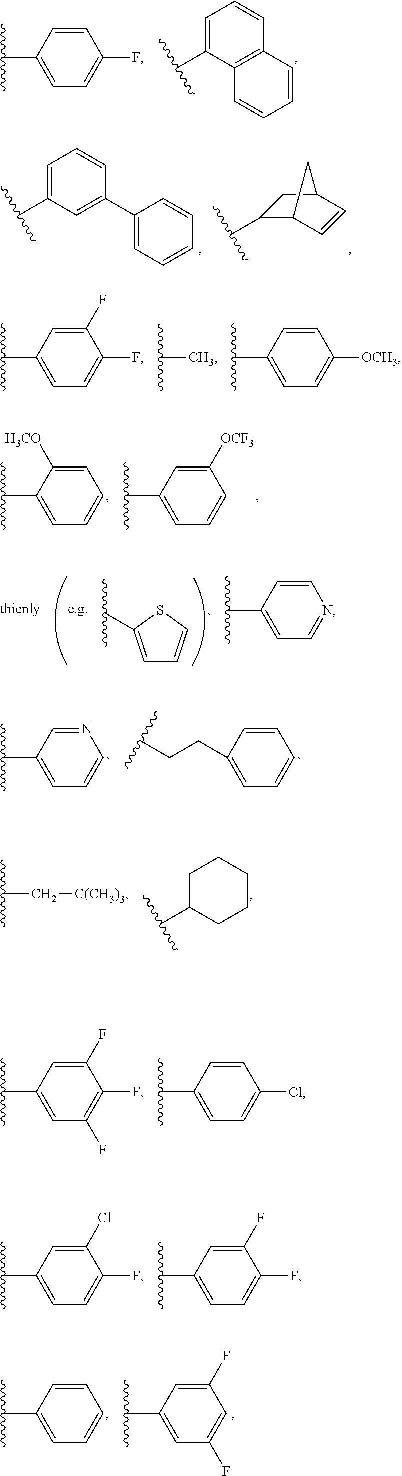

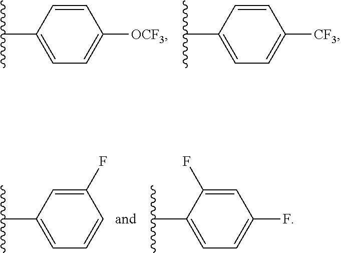

and wherein said R⁷ groups are substituted with 1 to 3 substituents independently selected from the group consisting of: —SF₅, —OSF₅, and —Si(R²⁴)₃ wherein each R²⁴ is the same or different alkyl group.

In another embodiment R⁶ is selected from the group consisting of: H, methyl, cyclopropyl, —C(O)CH₂CH₃, —CHF₂, —CF₃, —C(O)OCH₃, —CH₂OH, —CH₂OCH₃, —CH₂OCH(CH₃)₂, —C(O)OH, —C(CH₃)₃, —C(OH)(CH₃)₂, —C(O)CH₃, —CH(CH₃)OH, —CH₂C(OH)(CH₃)₂, —CH₂CH₂OH and

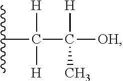

and R⁷ is selected from the group consisting of:

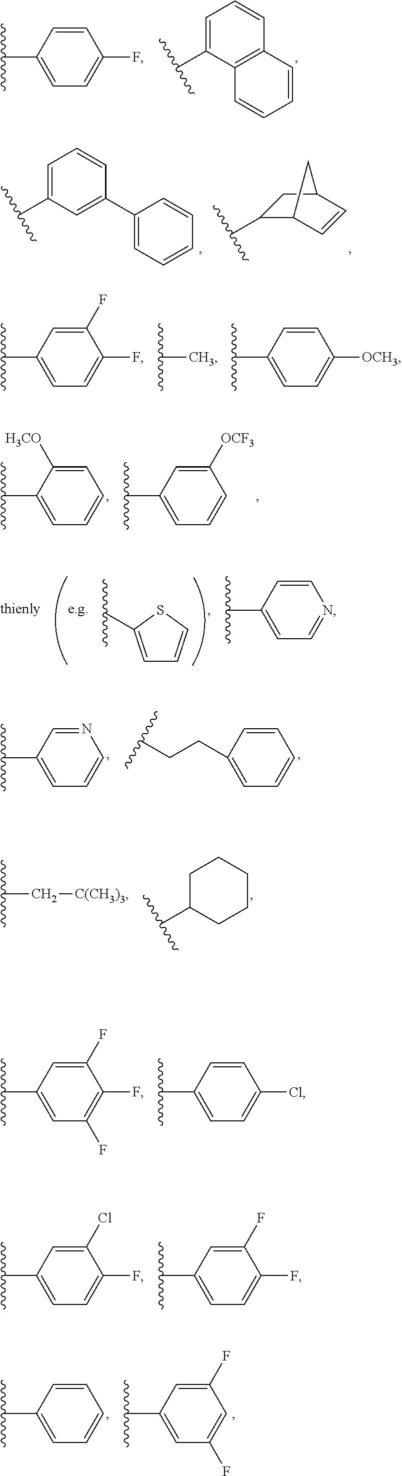

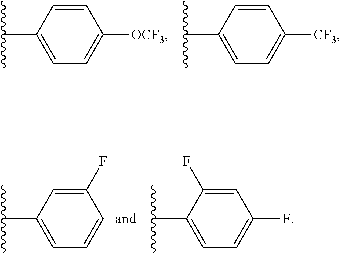

and wherein said R⁷ groups are substituted with 1 to 3 substituents independently selected from the group consisting of: —SF₅ and —OSF₅.

In another embodiment, R⁸ is H.

In another embodiment, R⁸ is alkyl.

In another embodiment, R⁸ is methyl.

In another embodiment, R¹⁰ is aryl.

In another embodiment, R¹⁰ is phenyl.

In another embodiment R¹⁰ is aryl substituted with 1 to 3 independently selected R²¹ moieties.

In another embodiment R¹⁰ is aryl substituted with 1 to 3 R²¹ moieties, wherein each R²¹ moiety is the same or different —OR¹⁵ group.

In another embodiment R¹⁰ is aryl substituted with 1 R²¹ moiety.

In another embodiment R¹⁰ is aryl substituted with one R²¹ moiety, and said R²¹ moiety is —OR¹⁵.

In another embodiment R¹⁰ is aryl substituted with one R²¹ moiety, said R²¹ moiety is —OR¹⁵, and said R¹⁵ is alkyl.

In another embodiment R¹⁰ is aryl substituted with one R²¹ moiety, said R²¹ moiety is —OR¹⁵, said R¹⁵ is alkyl, and said alkyl is methyl (i.e., said R²¹ moiety is —OCH₃).

In another embodiment R¹⁰ is phenyl substituted with 1 to 3 independently selected R²¹ moieties.

In another embodiment R¹⁰ is phenyl substituted with 1 to 3 R²¹ moieties, wherein each R²¹ moiety is the same or different —OR¹⁵ group.

In another embodiment R¹⁰ is phenyl substituted with 1 R²¹ moiety.

In another embodiment R¹⁰ is phenyl substituted with one R²¹ moiety, and said R²¹ moiety is —OR¹⁵.

In another embodiment R¹⁰ is phenyl substituted with one R²¹ moiety, said R²¹ moiety is —OR¹⁵, and said R¹⁵ is alkyl.

In another embodiment R¹⁰ is phenyl substituted with one R²¹ moiety, said R²¹ moiety is —OR¹⁵, said R¹⁵ is alkyl, and said alkyl is methyl (i.e., said R²¹ moiety is —OCH₃).

In another embodiment R¹⁰ is:

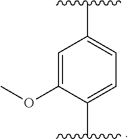

In another embodiment R¹⁰ is:

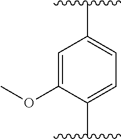

wherein the —R¹⁰—R⁹ moiety is:

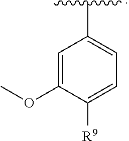

In another embodiment R¹⁰ is aryl substituted with 1 to 3 R²¹ moieties, wherein each R²¹ moiety is the same or different halo.

In another embodiment R¹⁰ is aryl substituted with 1 to 3 R²¹ moieties, wherein each R²¹ moiety is F.

In another embodiment R¹⁰ is aryl substituted with one R²¹ moiety, and said R²¹ moiety is halo.

In another embodiment R¹⁰ is aryl substituted with one R²¹ moiety, said R²¹ moiety is -halo, and said halo is F.

In another embodiment R¹⁰ is phenyl substituted with 1 to 3 R²¹ moieties, wherein each R²¹ moiety is the same or different halo.

In another embodiment R¹⁰ is phenyl substituted with 1 to 3 R²¹ moieties, wherein each R²¹ moiety is F.

In another embodiment R¹⁰ is phenyl substituted with one R²¹ moiety, and said R²¹ moiety is halo.

In another embodiment R¹⁰ is phenyl substituted with one R²¹ moiety, said R²¹ moiety is -halo, and said halo is F.

In another embodiment R¹⁰ is:

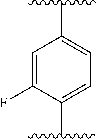

In another embodiment R¹⁰ is:

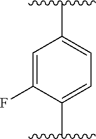

wherein the —R¹⁰—R⁹ moiety is:

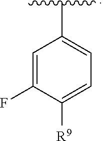

In another embodiment, R¹⁰ is unsubstituted heteroaryl.

In another embodiment R¹⁰ is unsubstituted heteroaryl wherein said heteroaryl is pyridyl.

In another embodiment R¹⁰ is:

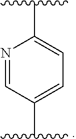

In another embodiment R¹⁰ is:

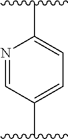

wherein the —R¹⁰—R⁹ moiety is:

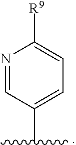

In another embodiment R¹⁰ is selected from the group consisting of:

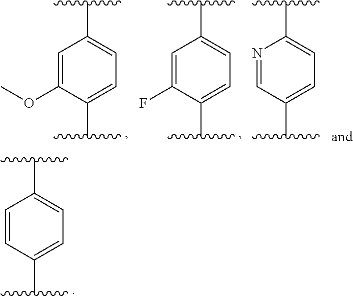

In another embodiment of this invention R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, and wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-3 substituents independently selected from the group consisting of the R²¹ groups.

In another embodiment, R⁹ is unsubstituted heteroaryl.

In another embodiment of this invention R⁹ is selected from the group consisting of heteroaryl and heteroaryl substituted with 1-3 R²¹ groups, and wherein each R²¹ is independently selected.

In another embodiment, R⁹ is heteroaryl which is substituted with 1-3 substituents which can be the same or different, each substituent being independently selected from the group consisting of halo, alkyl, CN, NH₂, NH(alkyl), N(alkyl)₂, hydroxy and alkoxy groups.

In another embodiment of this invention R⁹ is selected from the group consisting of imidazolyl and imidazolyl substituted with 1-3 R²¹ groups, and wherein each R²¹ is independently selected.

In another embodiment of this invention R⁹ is imidazolyl substituted with 1-3 R²¹ groups, and wherein each R²¹ is independently selected.

In another embodiment, R⁹ is imidazolyl substituted with 1-3 substituents independently selected from the group consisting of halo, alkyl, CN, NH₂, NH(alkyl), N(alkyl)₂, hydroxy and alkoxy groups.

In another embodiment of this invention R⁹ is selected from the group consisting of:

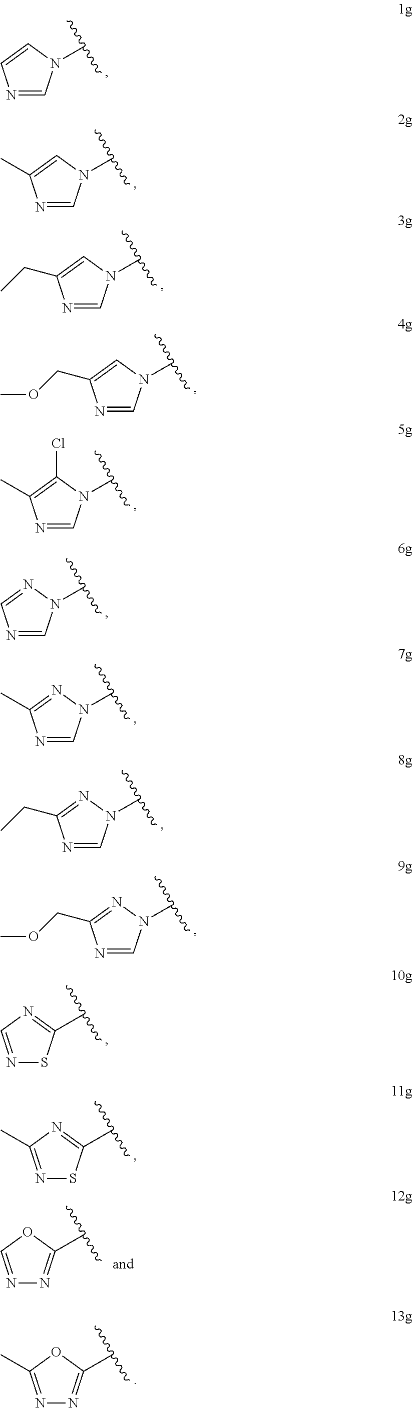

In another embodiment of this invention R⁹ is 1g. In another embodiment of this invention R⁹ is:

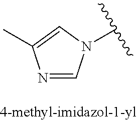

(i.e. 2g). In another embodiment of this invention R⁹ is 3g. In another embodiment of this invention R⁹ is 4g. In another embodiment of this invention R⁹ is 5g. In another embodiment of this invention R⁹ is 6g. In another embodiment of this invention R⁹ is 7g. In another embodiment of this invention R⁹ is 8g. In another embodiment of this invention R⁹ is 9g. In another embodiment of this invention R⁹ is 10g. In another embodiment of this invention R⁹ is 11g. In another embodiment of this invention R⁹ is 12g. In another embodiment of this invention R⁹ is 13g.

In another embodiment, R⁹ is imidazol-1-yl.

In another embodiment, R⁹ is 4-methyl-imidazol-1-yl.

In another embodiment, R⁹ is 5-chloro-4-methyl-imidazol-1-yl.

In another embodiment R⁹ is H.

In another embodiment R¹⁰ is selected from the group consisting of aryl and aryl substituted with one or more R²¹ groups, and R⁹ is selected from the group consisting of heteroaryl and heteroaryl substituted with one or more R²¹ groups, and wherein each R²¹ is independently selected.

In another embodiment R¹⁰ is selected from the group consisting of phenyl and phenyl substituted with 1-3 independently selected R²¹ groups, and R⁹ is selected from the group consisting of imidazolyl and imidazolyl substituted with 1-3 independently selected R²¹ groups.

In another embodiment R¹⁰ is phenyl substituted with 1-3 independently selected R²¹ groups, and R⁹ is selected from the group consisting of imidazolyl and imidazolyl substituted with 1-3 independently selected R²¹ groups.

In another embodiment R¹⁰ is selected from the group consisting of heteroaryl and heteroaryl substituted with 1-3 R²¹ groups, and the R⁹ group is selected from the group consisting of heteroaryl and heteroaryl substituted with 1-3 R²¹ groups, and wherein each R²¹ is independently selected.

In another embodiment R¹⁰ is selected from the group consisting of pyridyl and pyridyl substituted with 1-3 R²¹ groups, and the R⁹ group is selected from the group consisting of imidazolyl and imidazolyl substituted with 1-3 R²¹ groups, and wherein each R²¹ is independently selected.

In another embodiment R¹⁰ is pyridyl, and the R⁹ group is imidazolyl substituted with 1-3 R²¹ groups, and wherein each R²¹ is independently selected.

In another embodiment of this invention R¹⁰ is selected from the group consisting of 1A to 42A, and R⁹ is selected from the group consisting of 1g to 13g.

In another embodiment of this invention R¹⁰ is selected from the group consisting of 1A to 42A, and R⁹ is 2g.

In another embodiment of this invention R¹⁰ is selected from the group consisting of 1A to 42A, and R⁹ is H.

In another embodiment of this invention the R¹⁰—R⁹— moiety is selected from the group consisting of:

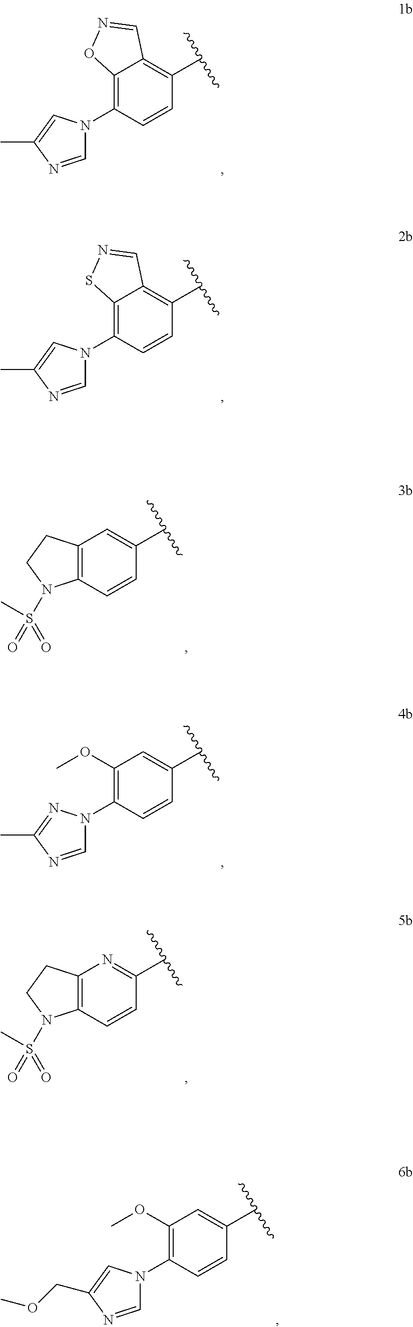

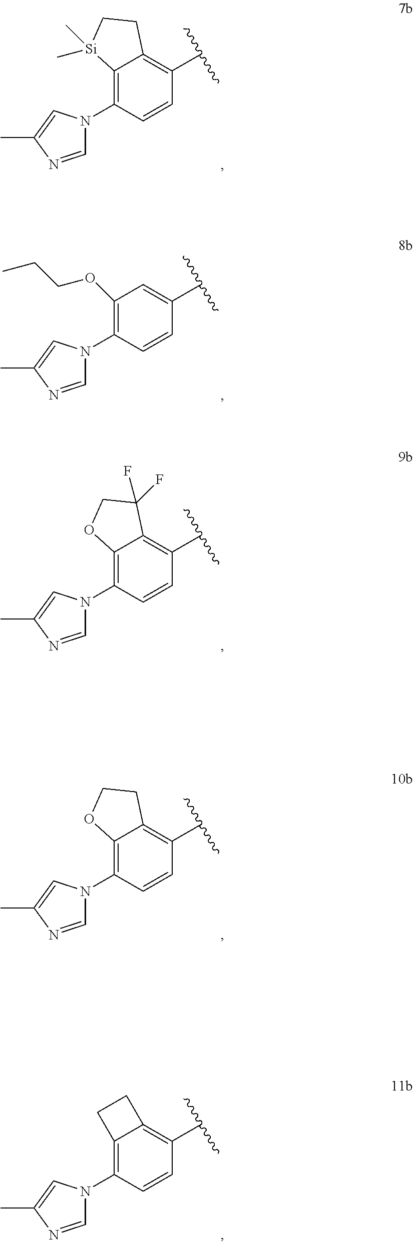

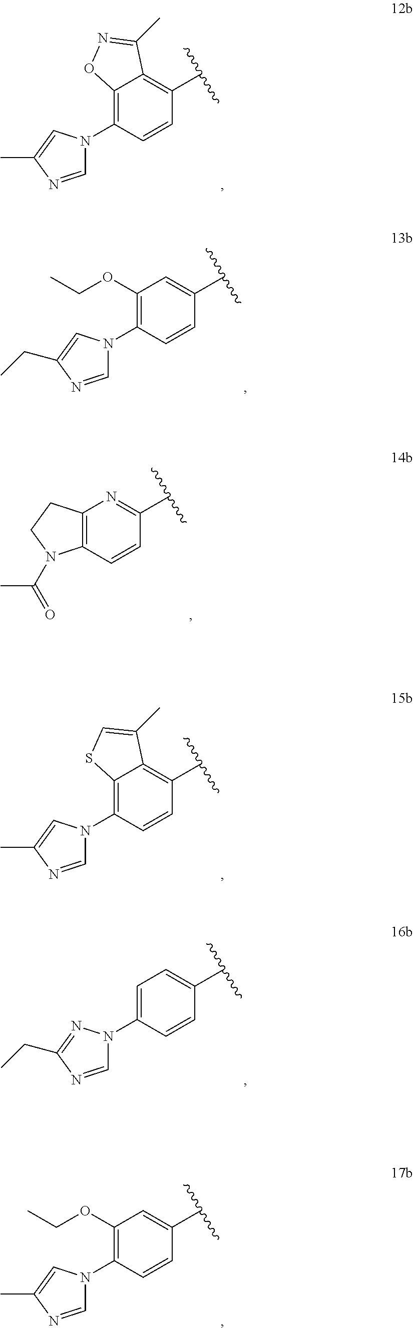

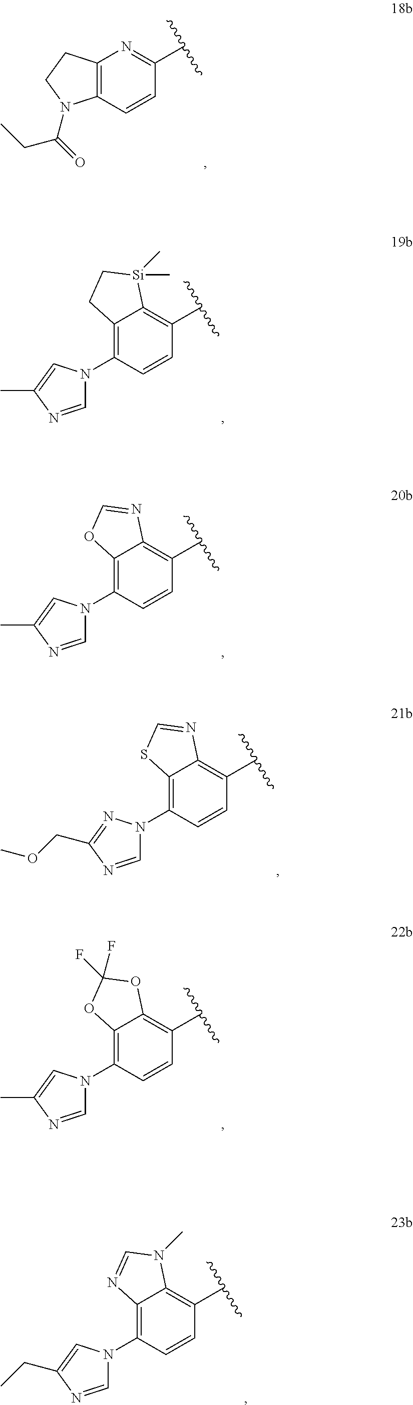

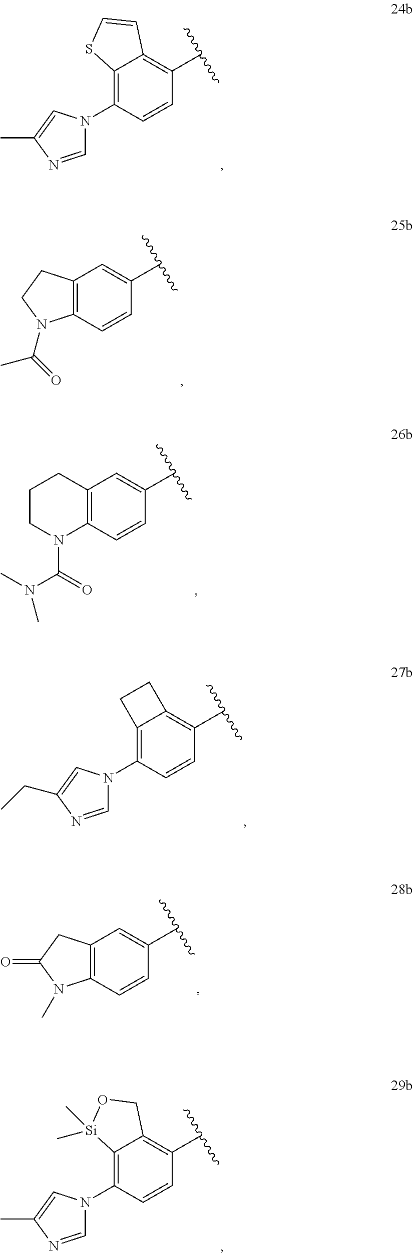

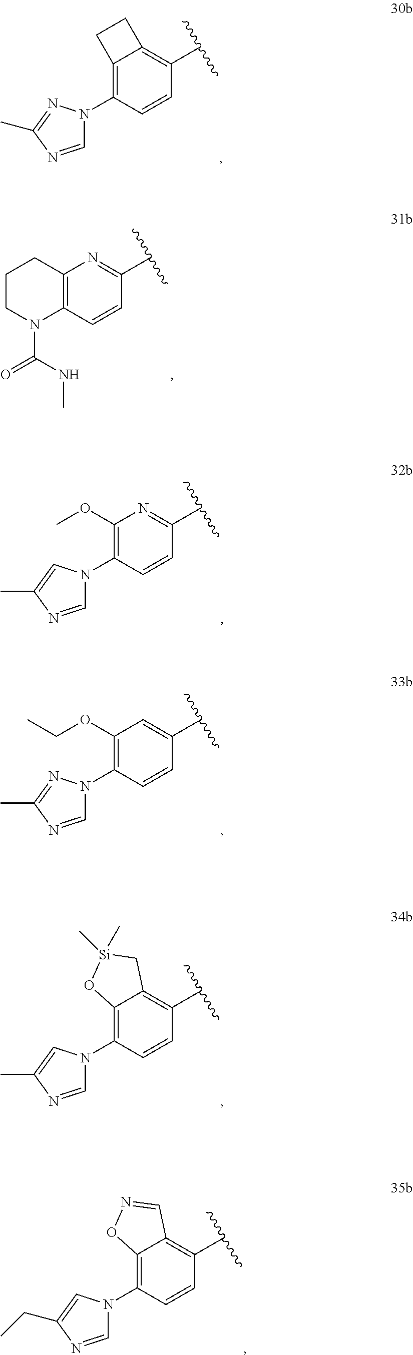

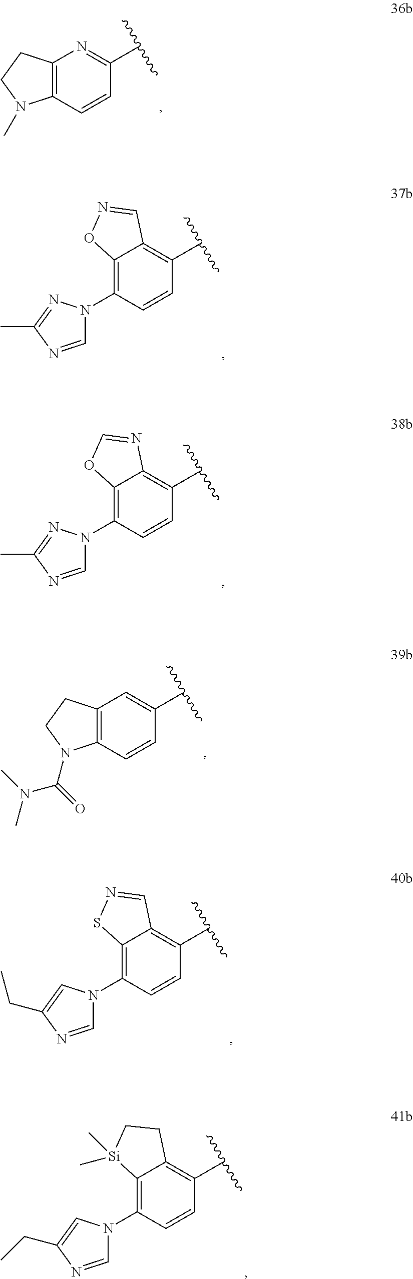

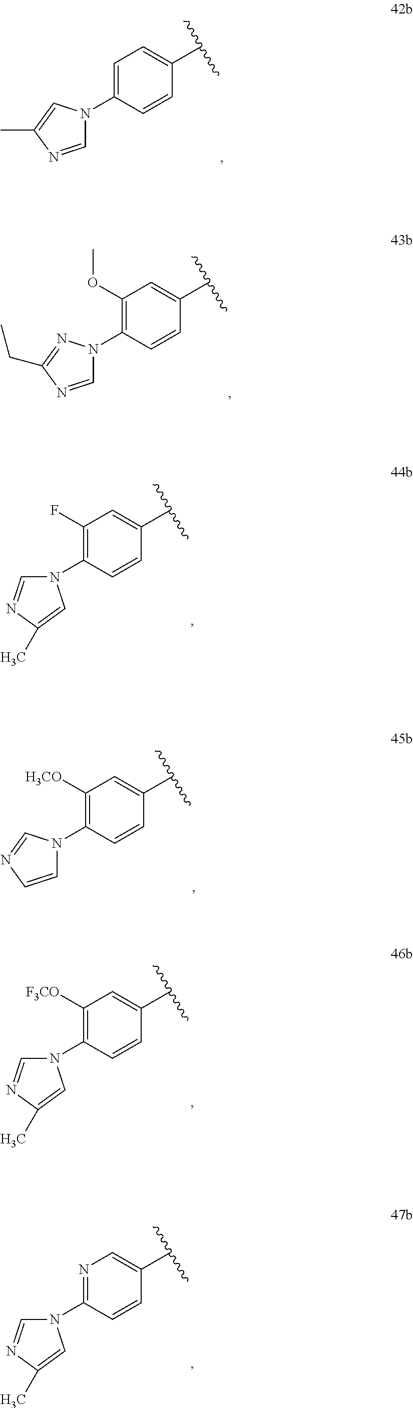

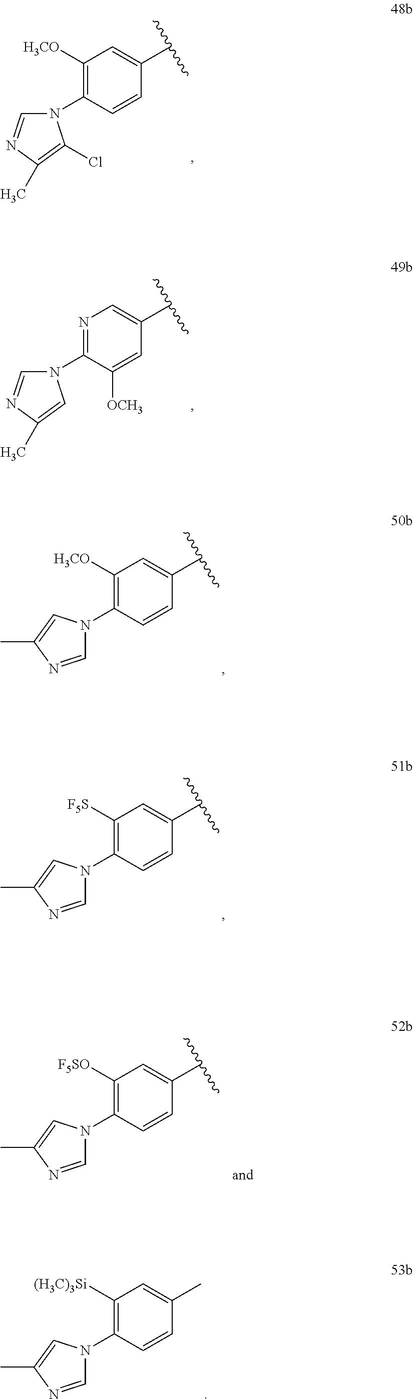

In another embodiment the R¹⁰—R⁹— moiety is 1b. In another embodiment the R¹⁰—R⁹— moiety is 2b. In another embodiment the R¹⁰—R⁹— moiety is 3b. In another embodiment the R¹⁰—R⁹— moiety is 4b. In another embodiment the R¹⁰—R⁹— moiety is 5b. In another embodiment the R¹⁰—R⁹— moiety is 6b. In another embodiment the R¹⁰—R⁹— moiety is 7b. In another embodiment the R¹⁰—R⁹— moiety is 8b. In another embodiment the R¹⁰—R⁹— moiety is 9b. In another embodiment the R¹⁰—R⁹— moiety is 10b. In another embodiment the R¹⁰—R⁹— moiety is 11b. In another embodiment the R¹⁰—R⁹— moiety is 12b. In another embodiment the R¹⁰—R⁹— moiety is 13b. In another embodiment the R¹⁰—R⁹— moiety is 14b. In another embodiment the R¹⁰—R⁹— moiety is 15b. In another embodiment the R¹⁰—R⁹— moiety is 16b. In another embodiment the R¹⁰—R⁹— moiety is 17b. In another embodiment the R¹⁰—R⁹— moiety is 18b. In another embodiment the R¹⁰—R⁹— moiety is 19b. In another embodiment the R¹⁰—R⁹— moiety is 20b. In another embodiment the R¹⁰—R⁹— moiety is 21b. In another embodiment the R¹⁰—R⁹— moiety is 22b. In another embodiment the R¹⁰—R⁹— moiety is 23b. In another embodiment the R¹⁰—R⁹— moiety is 24b. In another embodiment the R¹⁰—R⁹— moiety is 25b. In another embodiment the R¹⁰—R⁹— moiety is 26b. In another embodiment the R¹⁰—R⁹— moiety is 27b. In another embodiment the R¹⁰—R⁹— moiety is 28b. In another embodiment the R¹⁰—R⁹— moiety is 29b. In another embodiment the R¹⁰—R⁹— moiety is 30b. In another embodiment the R¹⁰—R⁹— moiety is 31b. In another embodiment the R¹⁰—R⁹— moiety is 32b. In another embodiment the R¹⁰—R⁹— moiety is 33b. In another embodiment the R¹⁰—R⁹— moiety is 34b. In another embodiment the R¹⁰—R⁹— moiety is 35b. In another embodiment the R¹⁰—R⁹— moiety is 36b. In another embodiment the R¹⁰—R⁹— moiety is 37b. In another embodiment the R¹⁰—R⁹— moiety is 38b. In another embodiment the R¹⁰—R⁹— moiety is 39b. In another embodiment the R¹⁰—R⁹— moiety is 40b. In another embodiment the R¹⁰—R⁹— moiety is 41b. In another embodiment the R¹⁰—R⁹— moiety is 42b. In another embodiment the R¹⁰—R⁹— moiety is 43b. In another embodiment the R¹⁰—R⁹— moiety is 44b. In another embodiment the R¹⁰—R⁹— moiety is 45b. In another embodiment the R¹⁰—R⁹— moiety is 46b. In another embodiment the R¹⁰—R⁹— moiety is 47b. In another embodiment the R¹⁰—R⁹— moiety is 48b. In another embodiment the R¹⁰—R⁹— moiety is 49b. In another embodiment the R¹⁰—R⁹— moiety is 50b. In another embodiment the R¹⁰—R⁹— moiety is 51b. In another embodiment the R¹⁰—R⁹— moiety is 52b. In another embodiment the R¹⁰—R⁹— moiety is 53b.

In another embodiment the R⁹—R¹⁰— moiety is:

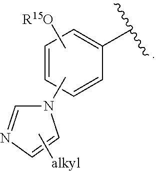

In another embodiment the R⁹—R¹⁰— moiety is:

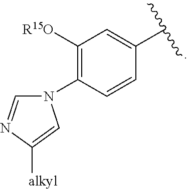

In another embodiment the R⁹—R¹⁰— moiety is:

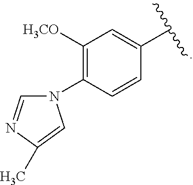

In another embodiment the R⁹—R¹⁰— moiety is:

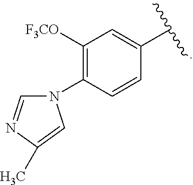

In another embodiment the R⁹—R¹⁰— moiety is:

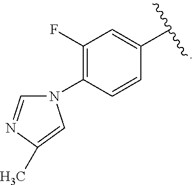

In another embodiment R⁹—R¹⁰— moiety is:

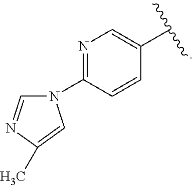

In another embodiment R⁹—R¹⁰— moiety is:

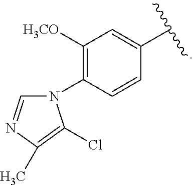

In another embodiment, the present application discloses a compound, or pharmaceutically acceptable salts, solvates, esters or prodrugs of said compound, said compound having the general structure shown in Formula:

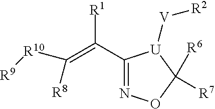

wherein the various moieties are defined above.

In another embodiment, the present application discloses a compound, or pharmaceutically acceptable salts, solvates, esters or prodrugs of said compound, said compound having the general structure shown in Formula:

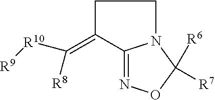

wherein the various moieties are defined above.

In another embodiment, the present application discloses a compound, or pharmaceutically acceptable salts, solvates, esters or prodrugs of said compound, said compound having the general structure shown in Formula:

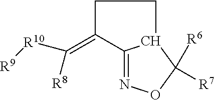

wherein the various moieties are defined above.

In another embodiment, the present application discloses a compound, or pharmaceutically acceptable salts, solvates, esters or prodrugs of said compound, said compound having the general structure shown in Formula:

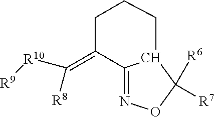

wherein the various moieties are defined above.

In another embodiment, the present application discloses a compound, or pharmaceutically acceptable salts, solvates, esters or prodrugs of said compound, said compound having the general structure shown in Formula:

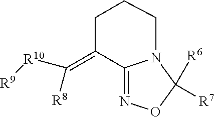

wherein the various moieties are defined above.

In another embodiment, this invention discloses a compound, or pharmaceutically acceptable salts, solvates, esters or prodrugs of said compound, said compound having the general structure shown in the formula:

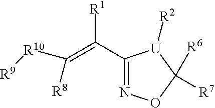

wherein U is N;

R¹ is H;

R² is alkyl;

R⁶ is H;

R⁷ is 3-(1,1′-biphenyl)-yl substituted with 1 to 3 groups independently selected from the group consisting of —SF₅ and —OSF₅;

R⁸ is H;

R¹⁰ is phenyl; and

R⁹ is imidazol-1-yl.

In another embodiment, this invention discloses a compound, or pharmaceutically acceptable salts, solvates, esters or prodrugs of said compound, said compound having the general structure shown in the formula:

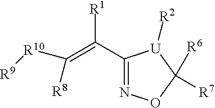

wherein U is N;

R¹ is H;

R² is alkyl;

R⁶ is H;

R⁷ is phenyl substituted with 1 to 3 groups independently selected from the group consisting of —SF₅ and —OSF₅;

R⁸ is H;

R¹⁰ is alkoxy-substituted phenyl; and

R⁹ is 4-methyl-imidazol-1-yl.

In another embodiment, this invention discloses a compound, or pharmaceutically acceptable salts, solvates, esters or prodrugs of said compound, said compound having the general structure shown in the formula:

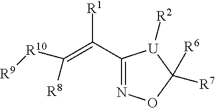

wherein U is N;

R¹ is H;

R² is alkyl;

R⁶ is H;

R⁷ is 4-fluoro-phen-1-yl substituted with 1 to 3 groups independently selected from the group consisting of —SF₅ and —OSF₅;

R⁸ is H;

R¹⁰ is phenyl; and

R⁹ is imidazol-1-yl.

In another embodiment, this invention discloses a compound, or pharmaceutically acceptable salts, solvates, esters or prodrugs of said compound, said compound having the general structure shown in the formula:

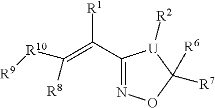

wherein U is N;

R¹ is H;

R² is methyl;

R⁶ is H;

R⁷ is 4-fluoro-phen-1-yl substituted with 1 to 3 groups independently selected from the group consisting of —SF₅ and —OSF₅;

R⁸ is H;

R¹⁰ is phenyl; and

R⁹ is 5-chloro-4-methyl-imidazol-1-yl.

In another embodiment, this invention discloses a compound, or pharmaceutically acceptable salts, solvates, esters or prodrugs of said compound, said compound having the general structure shown in the formula:

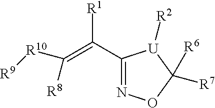

wherein U is N;

R¹ and R² are connected to form a 5-membered ring;

R⁶ is H;

R⁷ is 3-(1,1′-biphenyl)-yl substituted with 1 to 3 groups independently selected from the group consisting of —SF₅ and —OSF₅;

R⁸ is H;

R¹⁰ is phenyl; and

R⁹ is imidazol-1-yl.

In another embodiment, this invention discloses a compound, or pharmaceutically acceptable salts, solvates, esters or prodrugs of said compound, said compound having the general structure shown in the formula:

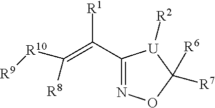

wherein U is N;

R¹ is H;

R² is alkyl;

R⁶ and R⁷ are connected to form a 5-membered spirocyclic ring wherein said spirocyclic ring is fused with a benzo ring, and said ring is substituted with 1 to 3 groups independently selected from the group consisting of —SF₅ and —OSF₅;

R⁸ is H;

R¹⁰ is phenyl; and

R⁹ is imidazol-1-yl.

In another embodiment, this invention discloses a compound, or pharmaceutically acceptable salts, solvates, esters or prodrugs of said compound, said compound having the general structure shown in the formula:

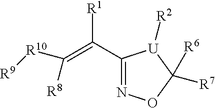

wherein U is N;

R¹ is H;

R² is alkoxyalkyl;

R⁶ is alkyl;

R⁷ is phenyl substituted with 1 to 3 groups independently selected from the group consisting of —SF₅ and —OSF₅;

R⁸ is H;

R¹⁰ is phenyl; and

R⁹ is imidazol-1-yl.

In another embodiment, this invention discloses a compound, or pharmaceutically acceptable salts, solvates, esters or prodrugs of said compound, said compound having the general structure shown in the formula:

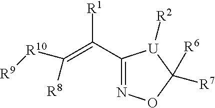

wherein U is N;

R¹ is H;

R² is arylalkyl;

R⁶ and R⁷ are connected to form a 5-membered spirocyclic ring wherein said spirocyclic ring is fused with a benzo ring, and said ring is substituted with 1 to 3 groups independently selected from the group consisting of —SF₅ and —OSF₅;

R⁸ is H;

R¹⁰ is alkoxy-substituted phenyl; and

R⁹ is imidazol-1-yl.

In another embodiment, this invention discloses a compound, or pharmaceutically acceptable salts, solvates, esters or prodrugs of said compound, said compound having the general structure shown in the formula:

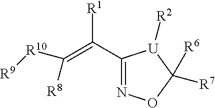

wherein U is N;

R¹ is H;

R² is (alkoxy)aryl-alkyl-;

R⁶ and R⁷ are connected to form a 5-membered spirocyclic ring wherein said spirocyclic ring is fused with a benzo ring, and said ring is substituted with 1 to 3 groups independently selected from the group consisting of —SF₅ and —OSF₅;

R⁸ is H;

R¹⁰ is (alkoxy-substituted)aryl; and

R⁹ is imidazol-1-yl.

In another embodiment, this invention discloses a compound, or pharmaceutically acceptable salts, solvates, esters or prodrugs of said compound, said compound having the general structure shown in the formula:

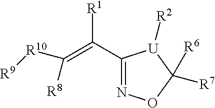

wherein U is N;

R¹ is H;

R² is alkoxy-alkyl-;

R⁶ is alkyl;

R⁷ is phenyl substituted with halo, and also substituted with substituted with 1 to 2 groups independently selected from the group consisting of —SF₅ and —OSF₅, wherein the total number of substituents on said phenyl is 2 or 3;

R⁸ is H;

R¹⁰ is (alkoxy-substituted)aryl; and

R⁹ is imidazol-1-yl.

In another embodiment, this invention discloses a compound, or pharmaceutically acceptable salts, solvates, esters or prodrugs of said compound, said compound having the general structure shown in the formula:

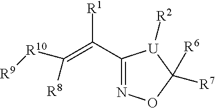

wherein U is N;

R¹ is H;

R² is alkoxy-alkyl-;

R⁶ is alkyl;

R⁷ is phenyl substituted with halo, and also substituted with substituted with 1 to 2 groups independently selected from the group consisting of —SF₅ and —OSF₅, wherein the total number of substituents on said phenyl is 2 or 3;

R⁸ is H;

R¹⁰ is (alkoxy-substituted)aryl; and

R⁹ is 4-alkyl-imidazol-1-yl.

In another embodiment, this invention discloses a compound, or pharmaceutically acceptable salts, solvates, esters or prodrugs of said compound, said compound having the general structure shown in the formula:

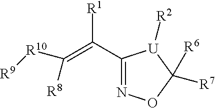

wherein U is N;

R¹ is H;

R² is alkoxy-alkyl-;

R⁶ and R⁷ are connected to form a 5-membered spirocyclic ring, wherein said ring is substituted with 1 to 3 groups independently selected from the group consisting of —SF₅ and —OSF₅;

R⁸ is H;

R¹⁰ is (alkoxy-substituted)aryl; and

R⁹ is 4-alkyl-imidazol-1-yl.

In another embodiment, this invention discloses a compound, or pharmaceutically acceptable salts, solvates, esters or prodrugs of said compound, said compound having the general structure shown in the formula:

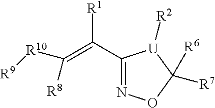

wherein U is N;

R¹ is H;

R² is alkoxy-alkyl-;

R⁶ and R⁷ are connected to form a 5-membered spirocyclic ring wherein said spirocyclic ring is fused with a benzo ring, wherein said ring is substituted with 1 to 3 groups independently selected from the group consisting of —SF₅ and —OSF₅;

R⁸ is H;

R¹⁰ is (alkoxy-substituted)aryl; and

R⁹ is 4-alkyl-imidazol-1-yl.

In another embodiment, this invention discloses a compound, or pharmaceutically acceptable salts, solvates, esters or prodrugs of said compound, said compound having the general structure shown in the formula:

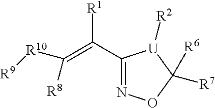

wherein U is N;

R¹ is H;

R² is alkoxy-alkyl-;

R⁶ and R⁷ are connected to form a 5-membered spirocyclic ring wherein said spirocyclic ring is fused with a benzo ring, wherein said ring is substituted with 1 to 3 groups independently selected from the group consisting of —SF₅ and —OSF₅;

R⁸ is H;

R¹⁰ is (alkoxy-substituted)aryl; and

R⁹ is 5-halo-4-alkyl-imidazol-1-yl.

In another embodiment, this invention discloses a compound, or pharmaceutically acceptable salts, solvates, esters or prodrugs of said compound, said compound having the general structure shown in the formula:

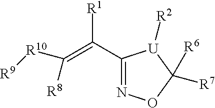

wherein U is N;

R¹ is H;

R² is alkoxy-alkyl-;

R⁶ and R⁷ are connected to form a 5-membered spirocyclic ring, wherein said ring is substituted with 1 to 3 groups independently selected from the group consisting of —SF₅ and —OSF₅;

R⁸ is H;

R¹⁰ is (alkoxy-substituted)aryl; and

R⁹ is 4-alkyl-imidazol-1-yl.

In another embodiment, this invention discloses a compound, or pharmaceutically acceptable salts, solvates, esters or prodrugs of said compound, said compound having the general structure shown in the formula:

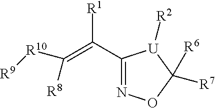

wherein U is N;

R¹ is H;

R² is alkoxy-alkyl-;

R⁶ is alkyl;

R⁷ is phenyl substituted with halo, and also substituted with substituted with 1 to 2 groups independently selected from the group consisting of —SF₅ and —OSF₅, wherein the total number of substituents on said phenyl is 2 or 3;

R⁸ is H;

R¹⁰ is (alkoxy-substituted)aryl; and

R⁹ is 5-halo-4-alkyl-imidazol-1-yl.

Other embodiments of this invention are directed to compounds of formula I selected from the group consisting of formulas II to IV wherein R⁶ and R⁷ do not form a spirocyclic moiety, and R⁶ and R⁷ are described in any of the embodiments above that are directed to R⁶ and R⁷, and R⁹ is selected from the group consisting of 1g to 13g, and R¹⁰ is selected from the group consisting of 1A to 42A.

Other embodiments of this invention are directed to compounds of formula I selected from the group consisting of formulas II to IV wherein R⁶ and R⁷ do not form a spirocyclic moiety, and R⁶ and R⁷ are described in any of the embodiments above that are directed to R⁶ and R⁷, and the R⁹—R¹⁰— moiety is selected from the group consisting of 1b to 53b.

Other embodiments of this invention are directed to compounds of formula I selected from the group consisting of formulas V to VIII wherein R⁹ is selected from the group consisting of 1g to 13g, and R¹⁰ is selected from the group consisting of 1A to 42A.

Other embodiments of this invention are directed to compounds of formula I selected from the group consisting of formulas V to VIII wherein the R⁹—R¹⁰— moiety is selected from the group consisting of 1b to 53b.

Other embodiments of this invention are directed to compounds of formula I selected from the group consisting of formulas IX to X wherein R⁷ is as described in any of the embodiments above that are directed to R⁷, and R⁹ is selected from the group consisting of 1g to 13g, and R¹⁰ is selected from the group consisting of 1A to 42A.

Other embodiments of this invention are directed to compounds of formula I selected from the group consisting of formulas II to IV wherein R⁷ is as described in any of the embodiments above that are directed to R⁷, and the R⁹—R¹⁰— moiety is selected from the group consisting of 1b to 53b.

Other embodiments are directed to any one of the above embodiments wherein R¹ and R⁸ are cis to each other instead of trans, that is the formulas above have the moiety:

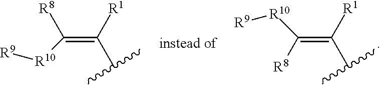

Other embodiments are directed to any one of the above embodiments wherein R⁶ has the stereochemistry:

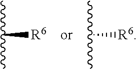

Other embodiments are directed to any one of the above embodiments wherein R⁷ has the stereochemistry:

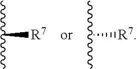

Thus other embodiments are directed to any one of the above embodiments wherein R⁶ and R⁷ have the stereochemistry

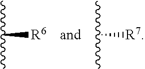

Other embodiments are directed to any one of the above embodiments wherein R⁶ and R⁷ have the stereochemistry

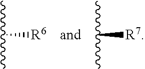

Representative compounds of the invention include, for example,

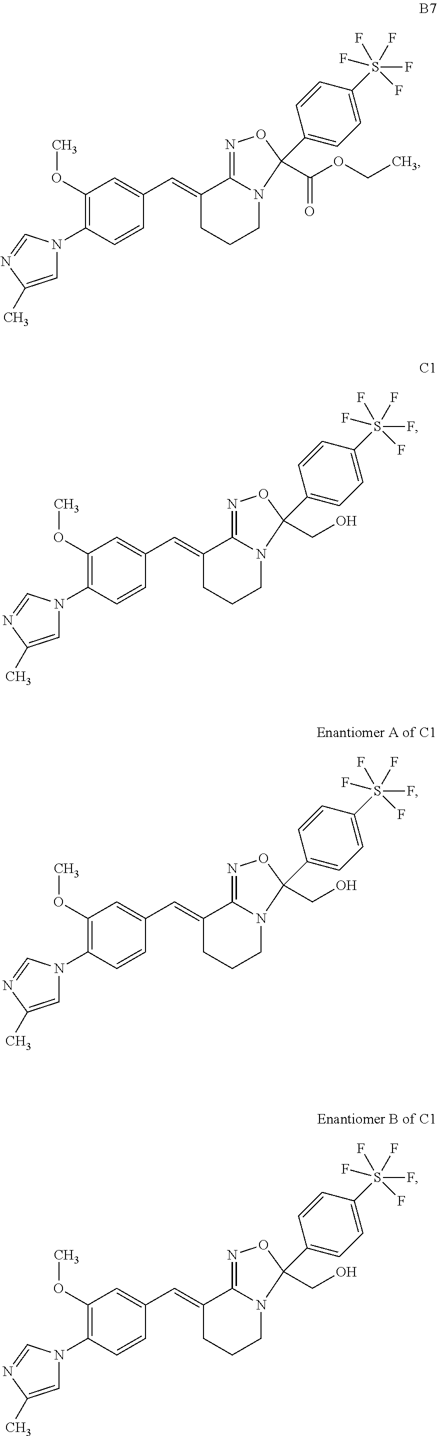

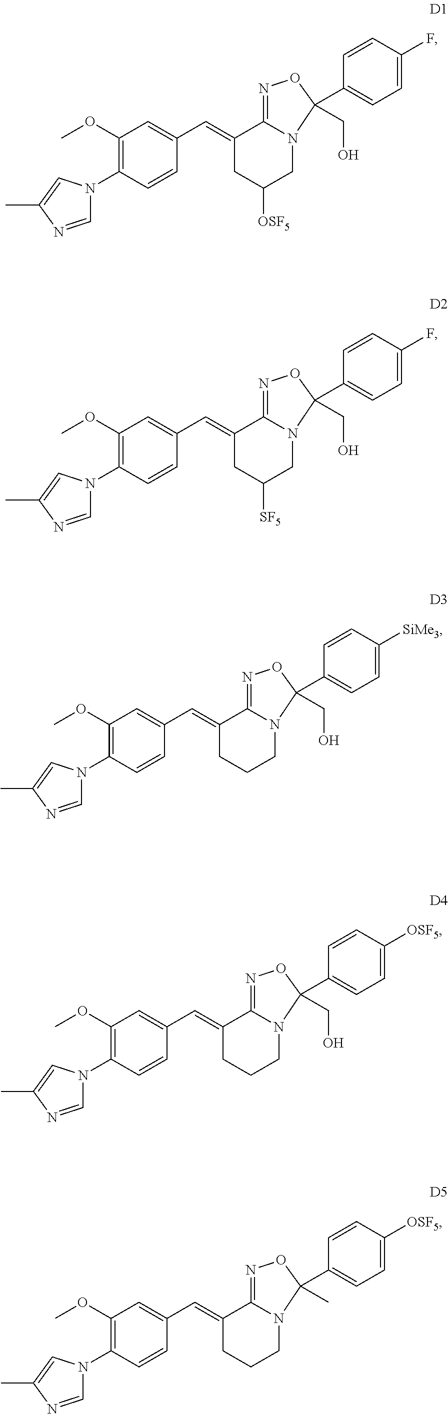

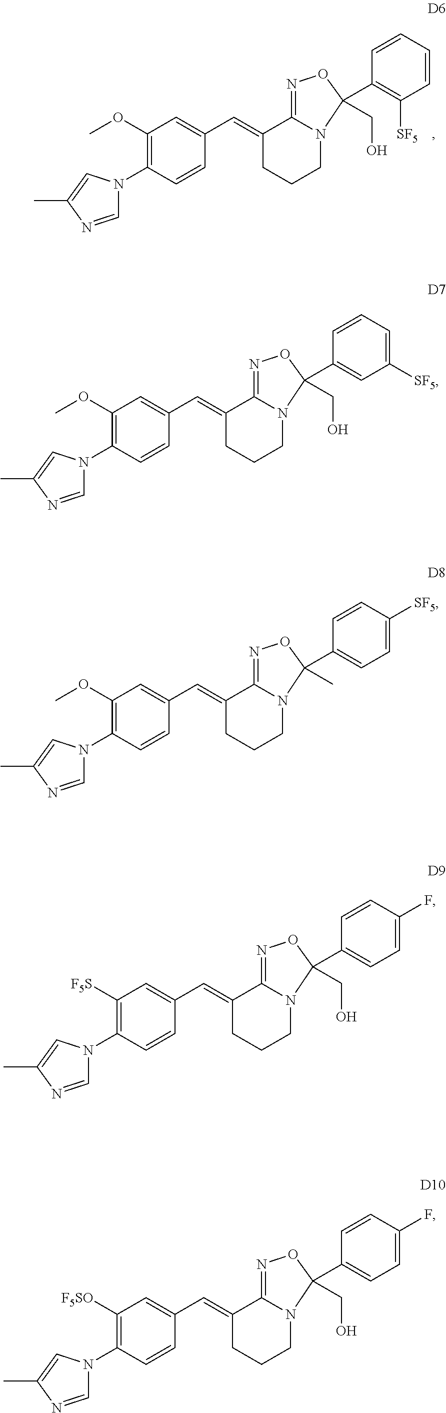

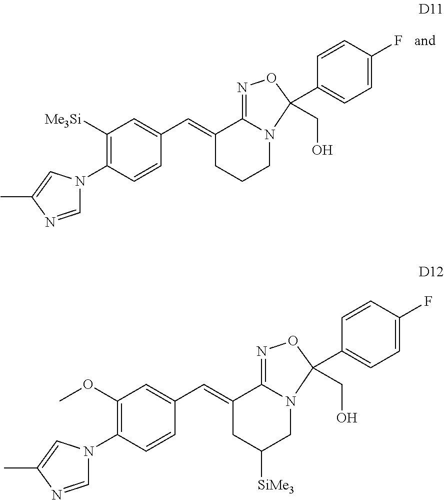

One embodiment of this invention is directed to compound B7. Another embodiment of this invention is directed to compound C1. Another embodiment of this invention is directed to Enantiomer A of compound C1. Another embodiment of this invention is directed to Enantiomer B of compound C1. Another embodiment of this invention is directed to compound D1. Another embodiment of this invention is directed to compound D2. Another embodiment of this invention is directed to compound D3. Another embodiment of this invention is directed to compound D4. Another embodiment of this invention is directed to compound D5. Another embodiment of this invention is directed to compound D6. Another embodiment of this invention is directed to compound D7. Another embodiment of this invention is directed to compound D8. Another embodiment of this invention is directed to compound D9. Another embodiment of this invention is directed to compound D10. Another embodiment of this invention is directed to compound D11. Another embodiment of this invention is directed to compound D12.

In the embodiments below Groups A, B and C are as defined as follows:

(1) Group A: II, III, IV, V (e.g., VA and VB), VI, VII, VIII, IX, and X;

(2) Group B: B7, C1 (e.g., Enantiomer A of C1 and Enantiomer B of C1), and D1 to D12; and

(3) Group C: B7, and C1 (e.g., Enantiomer A of C1, and Enantiomer B of C1).

Another embodiment of this invention is directed to compounds of formula I.

Another embodiment of this invention is directed to a compound of formula I selected from the group consisting of Group A.

Another embodiment of this invention is directed to a pharmaceutically acceptable salt of a compound of formula I. And in one example the salt is a salt of a compound selected from the group consisting of Group A. And in another example the salt is a salt of a compound selected from the group consisting of Group B. And in another example the salt is a salt of a compound selected from the group consisting of Group C.

Another embodiment of this invention is directed to a pharmaceutically acceptable ester of a compound of formula I. And in one example the ester is an ester of a compound selected from the group consisting of Group A. And in another example the ester is an ester of a compound selected from the group consisting of Group B. And in another example the ester is an ester of a compound selected from the group consisting of Group C.

Another embodiment of this invention is directed to a solvate of a compound of formula I. And in one example the solvate is a solvate of a compound selected from the group consisting of Group A. And in another example the solvate is a solvate of a compound selected from the group consisting of Group B. And in another example the solvate is a solvate of a compound selected from the group consisting of Group C.

Another embodiment of this invention is directed to a compound of formula I in pure and isolated form. And in one example the compound of formula I is selected from the group consisting of Group C.

Another embodiment of this invention is directed to a compound of formula I in pure form. And in one example the compound of formula I is selected from the group consisting of Group C.

Another embodiment of this invention is directed to a compound of formula I in isolated form. And in one example the compound of formula I is selected from the group consisting of Group C.

Another embodiment of this invention is directed to a compound of formula I selected from the group consisting of Group C.

Another embodiment of this invention is directed to a pharmaceutical composition comprising a therapeutically effective amount of at least one compound of Formula I, or a pharmaceutically acceptable salt, solvate, or ester thereof, and at least one pharmaceutically acceptable carrier.

Another embodiment of this invention is directed to a pharmaceutical composition comprising an effective amount of one or more (e.g., one) compounds of formula I and a pharmaceutically acceptable carrier.

Another embodiment of this invention is directed to a pharmaceutical composition comprising an effective amount of a pharmaceutically acceptable salt of one or more (e.g., one) compounds of formula I and a pharmaceutically acceptable carrier.

Another embodiment of this invention is directed to a pharmaceutical composition comprising an effective amount of a pharmaceutically acceptable ester of one or more (e.g., one) compounds of formula I and a pharmaceutically acceptable carrier.

Another embodiment of this invention is directed to a pharmaceutical composition comprising an effective amount of a solvate of one or more (e.g., one) compounds of formula I and a pharmaceutically acceptable carrier.

Another embodiment of this invention is directed to a pharmaceutical composition comprising an effective amount of one or more (e.g., one) compounds of formula I, and an effective amount of one or more (e.g., one) other pharmaceutically active ingredients (e.g., drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising a therapeutically effective amount of at least one compound of Formula I, or a pharmaceutically acceptable salt, solvate, or ester thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

Another embodiment of this invention is directed to a pharmaceutical composition comprising an effective amount of one or more (e.g., one) compounds of formula I, and effective amount of one or more BACE inhibitors, and a pharmaceutically acceptable carrier.

Another embodiment of this invention is directed to a pharmaceutical composition comprising an effective amount of one or more (e.g., one) compounds of formula I, and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds of formula I, and effective amount of one or more muscarinic antagonists (e.g., m₁ or m₂ antagonists), and a pharmaceutically acceptable carrier.

Another embodiment of this invention is directed to a pharmaceutical composition comprising an effective amount of one or more (e.g., one) compounds of formula I, and effective amount of Exelon (rivastigmine), and a pharmaceutically acceptable carrier.

Another embodiment of this invention is directed to a pharmaceutical composition comprising an effective amount of one or more (e.g., one) compounds of formula I, and effective amount of Cognex (tacrine), and a pharmaceutically acceptable carrier.

Another embodiment of this invention is directed to a pharmaceutical composition comprising an effective amount of one or more (e.g., one) compounds of formula I, and effective amount of a Tau kinase inhibitor, and a pharmaceutically acceptable carrier.

Another embodiment of this invention is directed to a pharmaceutical composition comprising an effective amount of one or more (e.g., one) compounds of formula I, and effective amount of one or more Tau kinase inhibitor (e.g., GSK3beta inhibitor, cdk5 inhibitor, ERK inhibitor), and a pharmaceutically acceptable carrier.

Another embodiment of this invention is directed to a pharmaceutical composition comprising an effective amount of one or more (e.g., one) compounds of formula I, and effective amount of one anti-Abeta vaccine (active immunization), and a pharmaceutically acceptable carrier.

Another embodiment of this invention is directed to a pharmaceutical composition comprising an effective amount of one or more (e.g., one) compounds of formula I, and effective amount of one or more APP ligands, and a pharmaceutically acceptable carrier.

Another embodiment of this invention is directed to a pharmaceutical composition comprising an effective amount of one or more (e.g., one) compounds of formula I, and effective amount of one or more agents that upregulate insulin degrading enzyme and/or neprilysin, and a pharmaceutically acceptable carrier.

Another embodiment of this invention is directed to a pharmaceutical composition comprising an effective amount of one or more (e.g., one) compounds of formula I, and effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe), and a pharmaceutically acceptable carrier.

Another embodiment of this invention is directed to a pharmaceutical composition comprising an effective amount of one or more (e.g., one) compounds of formula I, and effective amount of one or more fibrates (for example, clofibrate, Clofibride, Etofibrate, Aluminum Clofibrate), and a pharmaceutically acceptable carrier

Another embodiment of this invention is directed to a pharmaceutical composition comprising an effective amount of one or more (e.g., one) compounds of formula I, and effective amount of one or more LXR agonists, and a pharmaceutically acceptable carrier.

Another embodiment of this invention is directed to a pharmaceutical composition comprising an effective amount of one or more (e.g., one) compounds of formula I, and effective amount of one or more LRP mimics, and a pharmaceutically acceptable carrier.

Another embodiment of this invention is directed to a pharmaceutical composition comprising an effective amount of one or more (e.g., one) compounds of formula I, and effective amount of one or more 5-HT6 receptor antagonists, and a pharmaceutically acceptable carrier.

Another embodiment of this invention is directed to a pharmaceutical composition comprising an effective amount of one or more (e.g., one) compounds of formula I, and effective amount of one or more nicotinic receptor agonists, and a pharmaceutically acceptable carrier.

Another embodiment of this invention is directed to a pharmaceutical composition comprising an effective amount of one or more (e.g., one) compounds of formula I, and effective amount of one or more H3 receptor antagonists, and a pharmaceutically acceptable carrier.

Another embodiment of this invention is directed to a pharmaceutical composition comprising an effective amount of one or more (e.g., one) compounds of formula I, and effective amount of one or more histone deacetylase inhibitors, and a pharmaceutically acceptable carrier.

Another embodiment of this invention is directed to a pharmaceutical composition comprising an effective amount of one or more (e.g., one) compounds of formula I, and effective amount of one or more hsp90 inhibitors, and a pharmaceutically acceptable carrier.

Another embodiment of this invention is directed to a pharmaceutical composition comprising an effective amount of one or more (e.g., one) compounds of formula I, and effective amount of one or more m1 muscarinic receptor agonists, and a pharmaceutically acceptable carrier.

Another embodiment of this invention is directed to combinations, i.e., a pharmaceutical composition, comprising a pharmaceutically acceptable carrier, an effective (i.e., therapeutically effective) amount of one or more compounds of formula I, in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors.

Another embodiment of this invention is directed to a pharmaceutical composition comprising an effective amount of one or more (e.g., one) compounds of formula I, and effective amount of one or more 5-HT6 receptor antagonists mGluR1 or mGluR5 positive allosteric modulators or agonists, and a pharmaceutically acceptable carrier.

Another embodiment of this invention is directed to a pharmaceutical composition comprising an effective amount of one or more (e.g., one) compounds of formula I, and effective amount of one or more one mGluR2/3 antagonists, and a pharmaceutically acceptable carrier.

Another embodiment of this invention is directed to a pharmaceutical composition comprising an effective amount of one or more (e.g., one) compounds of formula I, and effective amount of one or more anti-inflammatory agents that can reduce neuroinflammation, and a pharmaceutically acceptable carrier.

Another embodiment of this invention is directed to a pharmaceutical composition comprising an effective amount of one or more (e.g., one) compounds of formula I, and effective amount of one or more Prostaglandin EP2 receptor antagonists, and a pharmaceutically acceptable carrier.

Another embodiment of this invention is directed to a pharmaceutical composition comprising an effective amount of one or more (e.g., one) compounds of formula I, and effective amount of one or more PAI-1 inhibitors, and a pharmaceutically acceptable carrier.

Another embodiment of this invention is directed to a pharmaceutical composition comprising an effective amount of one or more (e.g., one) compounds of formula I, and effective amount of one or more agents that can induce Abeta efflux such as gelsolin, and a pharmaceutically acceptable carrier.

Other embodiments of this invention are directed to any one of the above embodiments directed to pharmaceutical compositions wherein the compound of formula I is selected from the group consisting of Group A.

Other embodiments of this invention are directed to any one of the above embodiments directed to pharmaceutical compositions wherein the compound of formula I is selected from the group consisting of Group B.

Other embodiments of this invention are directed to any one of the above embodiments directed to pharmaceutical compositions wherein the compound of formula I is selected from the group consisting of Group C.

The compounds of formula I can be useful as gamma secretase modulators and can be useful in the treatment and prevention of diseases such as, for example, central nervous system disorders (such as Alzheimers disease and Downs Syndrome), mild cognitive impairment, glaucoma, cerebral amyloid angiopathy, stroke, dementia, microgliosis, brain inflammation, and olfactory function loss.

Another embodiment of this invention is directed to a method of treating a central nervous system disorder comprising administering a therapeutically effective amount of at least one compound of Formula I to a patient in need of such treatment.

Another embodiment of this invention is directed to a method of treating a central nervous system disorder comprising administering a therapeutically effective amount of a pharmaceutical composition comprising a therapeutically effective amount of at least one compound of Formula I, or a pharmaceutically acceptable salt, solvate, or ester thereof, and at least one pharmaceutically acceptable carrier.

Another embodiment of this invention is directed to a method of treating a central nervous system disorder comprising administering a therapeutically effective amount of a pharmaceutical composition comprising a therapeutically effective amount of at least one compound of Formula I, or a pharmaceutically acceptable salt, solvate, or ester thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

Another embodiment of this invention is directed to a method for modulating (including inhibiting, antagonizing and the like) gamma-secretase comprising administering an effective amount of one or more (e.g., one) compounds of formula I to a patient in need of such treatment.

Another embodiment of this invention is directed to a method for modulating (including inhibiting, antagonizing and the like) gamma-secretase, comprising administering an effective amount of a compound of formula I to a patient in need of treatment.

Another embodiment of this invention is directed to a method of treating one or more neurodegenerative diseases, comprising administering an effective amount of one or more (e.g., one) compounds of formula I to a patient in need of treatment.

Another embodiment of this invention is directed to a method of treating one or more neurodegenerative diseases, comprising administering an effective amount of a compound of formula I to a patient in need of treatment.

Another embodiment of this invention is directed to a method of inhibiting the deposition of amyloid protein (e.g., amyloid beta protein) in, on or around neurological tissue (e.g., the brain), comprising administering an effective amount of one or more (e.g., one) compounds of formula I to a patient in need of treatment.

Another embodiment of this invention is directed to a method of inhibiting the deposition of amyloid protein (e.g., amyloid beta protein) in, on or around neurological tissue (e.g., the brain), comprising administering an effective amount of a compound of formula I to a patient in need of treatment.

Another embodiment of this invention is directed to a method of treating Alzheimer's disease, comprising administering an effective amount of one or more (e.g., one) compounds of formula I to a patient in need of treatment.

Another embodiment of this invention is directed to a method of treating Alzheimer's disease, comprising administering an effective amount of a compound of formula I to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of one or more (e.g., one) compounds of formula I to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of a compound of formula I to a patient in need of treatment.

Another embodiment of this invention is directed to a method of treating mild cognitive impairment, comprising administering an effective amount of one or more (e.g., one) compounds of formula I to a patient in need of treatment.

Another embodiment of this invention is directed to a method of treating glaucoma, comprising administering an effective amount of one or more (e.g., one) compounds of formula I to a patient in need of treatment.

Another embodiment of this invention is directed to a method of treating cerebral amyloid angiopathy, comprising administering an effective amount of one or more (e.g., one) compounds of formula I to a patient in need of treatment.

Another embodiment of this invention is directed to a method of treating stroke, comprising administering an effective amount of one or more (e.g., one) compounds of formula I to a patient in need of treatment.

Another embodiment of this invention is directed to a method of treating dementia, comprising administering an effective amount of one or more (e.g., one) compounds of formula I to a patient in need of treatment.

Another embodiment of this invention is directed to a method of treating microgliosis, comprising administering an effective amount of one or more (e.g., one) compounds of formula I to a patient in need of treatment.

Another embodiment of this invention is directed to a method of treating brain inflammation, comprising administering an effective amount of one or more (e.g., one) compounds of formula I to a patient in need of treatment.

Another embodiment of this invention is directed to a method of treating olfactory function loss, comprising administering an effective amount of one or more (e.g., one) compounds of formula I to a patient in need of treatment.

Another embodiment of this invention is directed to a method of treating Downs syndrome, comprising administering an effective amount of one or more (e.g., one) compounds of formula I to a patient in need of treatment.

Another embodiment of this invention is directed to a method of treating Downs syndrome, comprising administering an effective amount of a compound of formula I to a patient in need of treatment.

Other embodiments of this invention are directed to any one of the above embodiments directed to methods of treating wherein the compound of formula I is selected from the group consisting of Group A.

Other embodiments of this invention are directed to any one of the above embodiments directed to methods of treating wherein the compound of formula I is selected from the group consisting of Group B.

Other embodiments of this invention are directed to any one of the above embodiments directed to methods of treating wherein the compound of formula I is selected from the group consisting of Group C.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula I and the administration of an effective amount of one or more (e.g., one) other pharmaceutical active ingredients (e.g., drugs). The compounds of formula I and the other drugs can be administered separately (i.e., each is in its own separate dosage form), or the compounds of formula I can be combined with the other drugs in the same dosage form.

Thus, other embodiments of this invention are directed to any one of the methods of treatment, or methods of inhibiting, described herein, wherein an effective amount of the compound of formula I is used in combination with an effective amount of one or more other pharmaceutically active ingredients (e.g., drugs). The other pharmaceutically active ingredients (i.e., drugs) are selected from the group consisting of: BACE inhibitors (beta secretase inhibitors), muscarinic antagonists (e.g., m₁ agonists or m₂ antagonists), cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminum Clofibrate); LXR agonists; LRP mimics; nicotinic receptor agonists; H3 receptor antagonists; histone deacetylase inhibitors; hsp90 inhibitors; m1 muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Another embodiment of this invention is directed to a method of treating Alzheimer's disease, comprising administering an effective amount of one or more (e.g., one) compounds of formula I, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of a compound of formula I, in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of one or more (e.g., one) compounds of formula I, in combination with an effective amount of one or more compounds selected from the group consisting of Aβ antibody inhibitors, gamma secretase inhibitors and beta secretase inhibitors.

Another embodiment of this invention is directed to a method of treating Alzheimer's disease, comprising administering an effective amount of one or more (e.g., one) compounds of formula I, in combination with an effective amount of one or more BACE inhibitors.

Another embodiment of this invention is directed to a method of treating Alzheimer's disease, comprising administering an effective amount of one or more compounds of formula I, in combination with an effective amount of Exelon (rivastigmine).

Another embodiment of this invention is directed to a method of treating Alzheimer's disease, comprising administering an effective amount of one or more compounds of formula I, in combination with an effective amount of Cognex (tacrine).

Another embodiment of this invention is directed to a method of treating Alzheimer's disease, comprising administering an effective amount of one or more compounds of formula I, in combination with an effective amount of a Tau kinase inhibitor.

Another embodiment of this invention is directed to a method of treating Alzheimer's disease, comprising administering an effective amount of one or more compounds of formula I, in combination with an effective amount of one or more Tau kinase inhibitor (e.g., GSK3beta inhibitor, cdk5 inhibitor, ERK inhibitor).

This invention also provides a method of treating Alzheimer's disease, comprising administering an effective amount of one or more compounds of formula I, in combination with an effective amount of one anti-Abeta vaccination (active immunization).

Another embodiment of this invention is directed to a method of treating Alzheimer's disease, comprising administering an effective amount of one or more compounds of formula I, in combination with an effective amount of one or more APP ligands.

Another embodiment of this invention is directed to a method of treating Alzheimer's disease, comprising administering an effective amount of one or more compounds of formula I, in combination with an effective amount of one or more agents that upregulate insulin degrading enzyme and/or neprilysin.

Another embodiment of this invention is directed to a method of treating Alzheimer's disease, comprising administering an effective amount of one or more compounds of formula I, in combination with an effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe).

This invention also provides a method of treating Alzheimer's disease, comprising administering an effective amount of one or more compounds of formula I, in combination with an effective amount of one or more fibrates (for example, clofibrate, Clofibride, Etofibrate, Aluminum Clofibrate).

Another embodiment of this invention is directed to a method of treating Alzheimer's disease, comprising administering an effective amount of one or more compounds of formula I, in combination with an effective amount of one or more LXR agonists.

Another embodiment of this invention is directed to a method of treating Alzheimer's disease, comprising administering an effective amount of one or more compounds of formula I, in combination with an effective amount of one or more LRP mimics.

Another embodiment of this invention is directed to a method of treating Alzheimer's disease, comprising administering an effective amount of one or more compounds of formula I, in combination with an effective amount of one or more 5-HT6 receptor antagonists.

Another embodiment of this invention is directed to a method of treating Alzheimer's disease, comprising administering an effective amount of one or more compounds of formula I, in combination with an effective amount of one or more nicotinic receptor agonists.

Another embodiment of this invention is directed to a method of treating Alzheimer's disease, comprising administering an effective amount of one or more compounds of formula I, in combination with an effective amount of one or more H3 receptor antagonists.

This invention also provides a method of treating Alzheimer's disease, comprising administering an effective amount of one or more compounds of formula I, in combination with an effective amount of one or more histone deacetylase inhibitors.

Another embodiment of this invention is directed to a method of treating Alzheimer's disease, comprising administering an effective amount of one or more compounds of formula I, in combination with an effective amount of one or more hsp90 inhibitors.

Another embodiment of this invention is directed to a method of treating Alzheimer's disease, comprising administering an effective amount of one or more compounds of formula I, in combination with an effective amount of one or more m1 muscarinic receptor agonists.

Another embodiment of this invention is directed to a method of treating Alzheimer's disease, comprising administering an effective amount of one or more compounds of formula I, in combination with an effective amount of one or more 5-HT6 receptor antagonists mGluR1 or mGluR5 positive allosteric modulators or agonists

Another embodiment of this invention is directed to a method of treating Alzheimer's disease, comprising administering an effective amount of one or more compounds of formula I, in combination with an effective amount of one or more mGluR2/3 antagonists.

Another embodiment of this invention is directed to a method of treating Alzheimer's disease, comprising administering an effective amount of one or more compounds of formula I, in combination with an effective amount of one or more anti-inflammatory agents that can reduce neuroinflammation.

Another embodiment of this invention is directed to a method of treating Alzheimer's disease, comprising administering an effective amount of one or more compounds of formula I, in combination with an effective amount of one or more Prostaglandin EP2 receptor antagonists.

Another embodiment of this invention is directed to a method of treating Alzheimer's disease, comprising administering an effective amount of one or more compounds of formula I, in combination with an effective amount of one or more PAI-1 inhibitors.

Another embodiment of this invention is directed to a method of treating Alzheimer's disease, comprising administering an effective amount of one or more compounds of formula I, in combination with an effective amount of one or more agents that can induce Abeta efflux such as gelsolin.

Another embodiment of this invention is directed to a method of treating Downs syndrome, comprising administering an effective amount of one or more (e.g., one) compounds of formula I, in combination with an effective amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Downs syndrome, comprising administering an effective amount of a compound of formula I, in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Other embodiments of this invention are directed to any one of the above embodiments directed to combination therapies (i.e., the above methods of treating wherein compounds of formula I are used in combination with other pharmaceutically active ingredients, i.e., drugs) wherein the compound of formula I is selected from the group consisting of Group A.

Other embodiments of this invention are directed to any one of the above embodiments directed to combination therapies (i.e., the above methods of treating wherein compounds of formula I are used in combination with other pharmaceutically active ingredients, i.e., drugs) wherein the compound of formula I is selected from the group consisting of Group B.

Other embodiments of this invention are directed to any one of the above embodiments directed to combination therapies (i.e., the above methods of treating wherein compounds of formula I are used in combination with other pharmaceutically active ingredients, i.e., drugs) wherein the compound of formula I is selected from the group consisting of Group C.

This invention also provides a kit comprising, in separate containers, in a single package, pharmaceutical compositions for use in combination, wherein one container comprises an effective amount of a compound of formula I in a pharmaceutically acceptable carrier, and another container (i.e., a second container) comprises an effective amount of another pharmaceutically active ingredient (as described above), the combined quantities of the compound of formula I and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase, or (e) mild cognitive impairment, or (f) glaucoma, or (g) cerebral amyloid angiopathy, or (h) stroke, or (i) dementia, or (j) microgliosis, or (k) brain inflammation, or (l) olfactory function loss.

This invention also provides a kit comprising, in separate containers, in a single package, pharmaceutical compositions for use in combination, wherein one container comprises an effective amount of a compound of formula I in a pharmaceutically acceptable carrier, and another container (i.e., a second container) comprises an effective amount of another pharmaceutically active ingredient (as described above), the combined quantities of the compound of formula I and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Other embodiments of this invention are directed to any one of the above embodiments directed to kits wherein the compound of formula I is selected from the group consisting of Group A.

Other embodiments of this invention are directed to any one of the above embodiments directed to kits wherein the compound of formula I is selected from the group consisting of Group B.

Other embodiments of this invention are directed to any one of the above embodiments directed to kits wherein the compound of formula I is selected from the group consisting of Group C.

Examples of cholinesterase inhibitors are tacrine, donepezil, rivastigmine, galantamine, pyridostigmine and neostigmine, with tacrine, donepezil, rivastigmine and galantamine being preferred.

Examples of m₁ antagonists are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US2005/0119227 published Jun. 2, 2005 (see also WO2005/016876 published Feb. 24, 2005), US2005/0043290 published Feb. 24, 2005 (see also WO2005/014540 published Feb. 17, 2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published Feb. 23, 2006 (see also WO2006/014762 published Feb. 9, 2006), WO2006/014944 published Feb. 9, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO2006/138217 published Dec. 28, 2006 (see also US2006/0287294 published Dec. 21, 2006), US2007/0099898 published May 3, 200 (see also WO2007/050721 published May 3, 2007), WO2007/053506 published May 10, 2007 (see also US2007/099875 published May 3, 2007), U.S. application Ser. No. 11/759,336 filed Jun. 7, 2007, U.S. Application Ser. No. 60/874,362 filed Dec. 12, 2006, and U.S. Application Ser. No. 60/874,419 filed Dec. 12, 2006, the disclosures of each being incorporated herein by reference thereto.

As used above, and throughout this disclosure, the following terms, unless otherwise indicated, shall be understood to have the following meanings:

“TBAF” means tetrabutyl ammonium fluoride

“At least one” means one or more than one, for example, 1, 2 or 3, or in another example, 1 or 2, or in another example 1.

“One or more” with reference to the use of the compounds of this invention means that one or more than one compound is used, for example, 1, 2 or 3, or in another example, 1 or 2, or in another example 1.

“Patient” includes both human and animals.

“Mammal” means humans and other mammalian animals.

It is noted that the carbons of formula I and other formulas herein may be replaced with 1 to 3 silicon atoms so long as all valency requirements are satisfied.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl, aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Carbocyclic” means a non-aromatic saturated or unsaturated mono- or multicyclic ring system comprising about 3 to about 10 carbon atoms, preferably about 5 to about 10 carbon atoms. Carbocyclic rings include cycloalkyl rings and cycloalkenyl rings as defined below. Thus, examples of carbocyclic rings include bicyclic rings, such as, for example, norbornyl, adamantly, norbornenyl, and

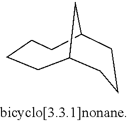

The carbocyclic rings are optionally substituted with one or more independently selected “ring system substituents” as defined below.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” (or “carbocyclenyl”) means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine. “Halo” refers to fluoro, chloro, bromo or iodo.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

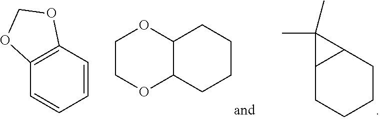

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” may also mean a single moiety (e.g., carbonyl) which simultaneously replaces two available hydrogens on the same carbon atom on a ring system. Example of such moiety is pyrrolidone:

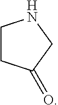

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” may also mean a single moiety (e.g., carbonyl) which simultaneously replaces two available hydrogens on the same carbon atom on a ring system. Example of such moiety is pyrrolidinone:

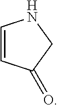

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

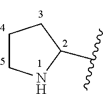

there is no —OH attached directly to carbons marked 2 and 5.

It should also be noted that tautomeric forms such as, for example, the moieties:

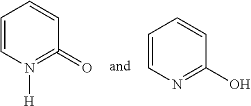

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of Formula I or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula I or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C1-C2)alkyl and piperidino-, pyrrolidino- or morpholino(C₂-C₃)alkyl, and the like.

Similarly, if a compound of Formula I contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula I incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” with reference to the amount of a compound of formula I, or another drug, used in a pharmaceutical composition, method of treatment or kit, means a therapeutically effective amount.

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compounds of Formula I, and salts, solvates, esters and prodrugs thereof, may exist in their tautomeric form (for example, as an amide, enol, keto or imino ether). All such tautomeric forms are contemplated herein as part of the present invention.

The compounds of Formula I may contain asymmetric or chiral centers, and, therefore, exist in different stereoisomeric forms. It is intended that all stereoisomeric forms of the compounds of Formula I as well as mixtures thereof, including racemic mixtures, form part of the present invention. In addition, the present invention embraces all geometric and positional isomers. For example, if a compound of Formula I incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula I may be atropisomers (e.g., substituted biaryls) and are considered as part of this invention. Enantiomers can also be separated by use of chiral HPLC column.

It is also possible that the compounds of Formula I may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula I incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl and ¹²³I, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Certain isotopically-labelled compounds of Formula (I) can be useful for medical imaging purposes. E.g., those labeled with positron-emitting isotopes like ¹¹C or ¹⁸F can be useful for application in Positron Emission Tomography (PET) and those labeled with gamma ray emitting isotopes like ¹²³I can be useful for application in Single photon emission computed tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half lives (T½>1 day), can generally be prepared by following procedures analogous to those disclosed in the Schemes and/or in the Examples herein below, by substituting an appropriate isotopically labeled reagent for a non-isotopically labeled reagent.

Polymorphic forms of the compounds of Formula I, and of the salts, solvates, esters and prodrugs of the compounds of Formula I, are intended to be included in the present invention.

The compounds according to the invention can have pharmacological properties; in particular, the compounds of Formula I can be modulators of gamma secretase (including inhibitors, antagonists and the like).

More specifically, the compounds of Formula I can be useful in the treatment of a variety of disorders of the central nervous system including, for example, including, but not limited to, Alzheimer's disease, AIDS-related dementia, Parkinson's disease, amyotrophic lateral sclerosis, retinitis pigmentosa, spinal muscular atrophy and cerebellar degeneration and the like.

Another aspect of this invention is a method of treating a mammal (e.g., human) having a disease or condition of the central nervous system by administering a therapeutically effective amount of at least one compound of Formula I, or a pharmaceutically acceptable salt, solvate, ester or prodrug of said compound to the mammal.

A preferred dosage is about 0.001 to 500 mg/kg of body weight/day of the compound of Formula I. An especially preferred dosage is about 0.01 to 25 mg/kg of body weight/day of a compound of Formula I, or a pharmaceutically acceptable salt or solvate of said compound.

The compounds of this invention may also be useful in combination (administered together or sequentially) with one or more additional agents listed above.

The compounds of this invention may also be useful in combination (administered together or sequentially) with one or more compounds selected from the group consisting of Aβ antibody inhibitors, gamma secretase inhibitors and beta secretase inhibitors.

If formulated as a fixed dose, such combination products employ the compounds of this invention within the dosage range described herein and the other pharmaceutically active agent or treatment within its dosage range.

Accordingly, in an aspect, this invention includes combinations comprising an amount of at least one compound of Formula I, or a pharmaceutically acceptable salt, solvate, ester or prodrug thereof, and an amount of one or more additional agents listed above wherein the amounts of the compounds/treatments result in desired therapeutic effect.

The pharmacological properties of the compounds of this invention may be confirmed by a number of pharmacological assays. Certain assays are exemplified later in this document.

This invention is also directed to pharmaceutical compositions which comprise at least one compound of Formula I, or a pharmaceutically acceptable salt, solvate, ester or prodrug of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula I, or a pharmaceutically acceptable salt, solvate, ester or prodrug of said compound and a pharmaceutically acceptable carrier, vehicle or diluent.

Yet another aspect of this invention is a kit comprising an amount of at least one compound of Formula I, or a pharmaceutically acceptable salt, solvate, ester or prodrug of said compound and an amount of at least one additional agent listed above, wherein the amounts of the two or more ingredients result in desired therapeutic effect.

The invention disclosed herein is exemplified by the following preparations and examples which should not be construed to limit the scope of the disclosure. Alternative mechanistic pathways and analogous structures will be apparent to those skilled in the art. Reagents and reaction conditions can be changed according to the knowledge of those skilled in the art.

Where NMR data are presented, ¹H spectra were obtained on either a Varian VXR-200 (200 MHz, ¹H), Varian Gemini-300 (300 MHz) or XL-400 (400 MHz) and are reported as ppm down field from Me₄Si with number of protons, multiplicities, and coupling constants in Hertz indicated parenthetically. Where LC/MS data are presented, analyses was performed using an Applied Biosystems API-100 mass spectrometer and Shimadzu SCL-10A LC column: Altech platinum C18, 3 micron, 33 mm×7 mm ID; gradient flow: 0 min—10% CH₃CN, 5 min—95% CH₃CN, 7 min—95% CH₃CN, 7.5 min—10% CH₃CN, 9 min—stop. The observed parent ion is given.

The following solvents and reagents may be referred to by their abbreviations in parenthesis:
DCM: dichloromethane (CH₂Cl₂)
DEA means diethylamine
DMF means N,N-dimethylformamide.
ethyl acetate: AcOEt or EtOAc
ethanol: EtOH
grams: g
high resolution mass spectrometry: HRMS
liquid chromatography mass spectrometry: LCMS
methanol: MeOH
microliters: μl
milligrams: mg
milliliters: mL
millimoles: mmol
nuclear magnetic resonance spectroscopy: NMR
SM: Starting Material
Thin layer chromatography: TLC
t-BU: tert-butyl
triethylamine: Et₃N or TEA
rt or r.t. means room temperature (ambient), about 25° C.

### EXAMPLES

### Example 1

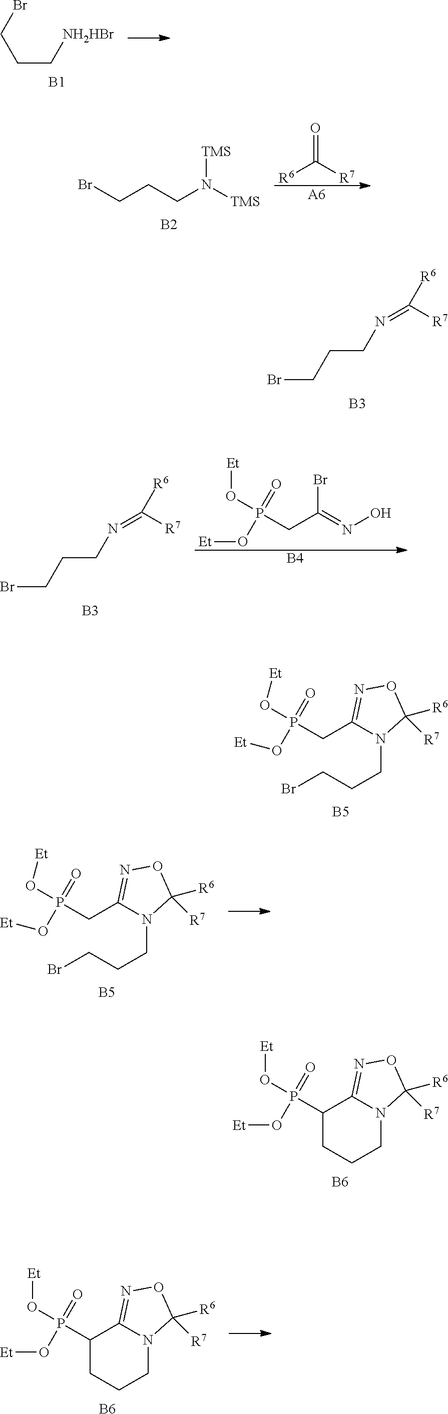

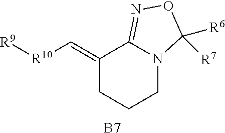

### Example 1, Step 1

Triethylamine (10.5 mL) was added slowly to a stirred suspension of B1 (5 g) in 66 mL of anhydrous DCM at 0° C. under nitrogen atmosphere. A solution of chlorotrimethylsilane (6.4 mL) in 12 mL in anhydrous DCM was added slowly to the above suspension. The reaction mixture was stirred at r.t. overnight before filtration to remove precipitate. The filtrate was evaporated and the residue oil was redissolved in 150 mL diethyl ether, stirred for 15 min, filtered and concentrated to give 5.7 g of B2.

### Example 1, Step 2

A catalytic amount of trimethylsilyl trifluoromethanesulfonate was added to a stirred mixture of B2 (2.8 g) and A6 (2.0 g, R⁷=p-SF₅-Phenyl and R⁶=carboethoxyl) in 50 mL of anhydrous DCE at r.t. under nitrogen atmosphere. The reaction mixture was refluxed for 4 h before cooled to r.t. and sequentially washed with cold NaHCO₃:water (1:1) and cold half-saturated brine. The organic phase was dried over anhydrous sodium sulfate, filtered and solvent removed to give 2.8 g of B3 (R⁷=p-SF₅-Phenyl and R⁶=carboethoxyl).

### Example 1, Step 3

A solution of B3 (R⁷=p-SF₅-Phenyl and R⁶=carboethoxyl) (2.8 g, 1 equiv.) in 30 mL of anhydrous DMF was slowly added to a solution of B4 (2 equiv. obtained following a reference procedure: Tsuge, Otohiko; Kanemasa, Shuji; Suga, Hiroyuki; Nakagawa, Norihiko Bulletin of the Chemical Society of Japan (1987), 60(7), 2463-73) in 100 mL of anhydrous DMF at 0° C. under nitrogen atmosphere. A solution of TEA (1.4 g, 2 equiv.) in 20 mL of anhydrous DMF was slowly added to the above reaction mixture. The reaction mixture was stirred at r.t. overnight before dilution with 20 mL of diethyl ether and 20 mL half-saturated brine. The aqueous phase was extracted with EtOAC:hexane (7:3). The organic phase was washed with half-saturated brine then, dried over anhydrous sodium sulfate. The solvent was evaporated and the residue was purified via a flash silica gel column eluted with Hexane/EtOAc to give B5 (1.2 g, R⁷=p-SF₅-Phenyl and R⁶=carboethoxyl).

### Example 1, Step 4

A solution of t-BuOK (1M in THF, 1.74 ml) in 54 mL of anhydrous THF was added dropwise to a stirred solution of B5 (1.02 g, R⁷=p-SF₅-Phenyl and R⁶=carboethoxyl) in 40 mL of anhydrous THF at −65° C. under nitrogen atmosphere. The reaction mixture was stirred between −65° C. and −40° C. until SM was consumed. The reaction mixture was quenched with iced brine, and extracted with EtOAc. The organic phase was washed with NH₄Cl and brine, dried over anhydrous magnesium sulfate, filtered and solvent evaporated. The residue was purified by a flash silica gel column and eluted with hexane/EtOAc to give 0.42 g of B6 (R⁷=p-SF₅-Phenyl and R⁶=carboethoxyl).

### Example 1, Step 5

A solution of t-BuOK (1M in THF, 0.80 ml) was added dropwise to a stirred mixture of B6 (R⁷=p-SF₅-Phenyl and R⁶=carboethoxyl, 0.41 g) and A1

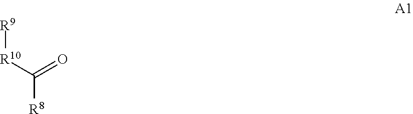

(157 mg, R¹⁰=3-MeO-Phenyl, R⁹=4-(4-Methyl-imidazol-1-yl) and R⁸═H) in 10 mL of anhydrous THF at −70° C. under nitrogen atmosphere. The reaction mixture was stirred between −70° C. and −30° C. until starting material were consumed. The reaction was quenched with iced brine, and extracted with EtOAc. The organic phase was washed with aqueous NH₄Cl and brine, dried over anhydrous magnesium sulfate, filtered and solvent evaporated. The residue was purified by a flash silica gel column and eluted with DCM/MeOH to give 0.38 g B7 (R⁷=p-SF₅-Phenyl, R⁶=carboethoxyl, R¹⁰=3-MeO-Phenyl, R⁹=4-(4-Methyl-imidazol-1-yl)):

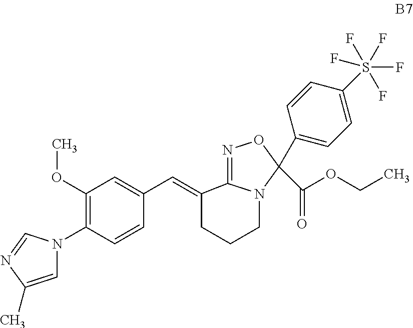

(Retention Time 3.6, Observed mass 599)

### Example 2

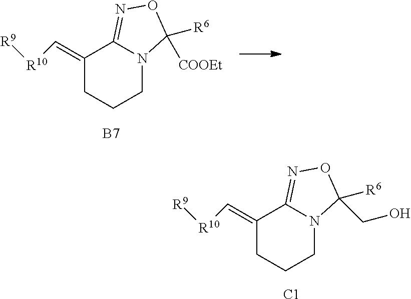

Solid sodium borohydride (100 mg) was added to a stirred solution of B7 (400 mg; R⁷=p-SF₅-Phenyl and R⁶=carboethoxyl, R¹⁰=3-MeO-Phenyl, R⁹=4-(4-Methyl-imidazol-1-yl)) in 30 mL of MeOH:THF (1:10) at 0° C. under nitrogen atmosphere. The reaction mixture was stirred at 0° C. for 1 h and then at r.t. for 1 hr, quenched with iced brine, and extracted with EtOAc. The organic phase was dried over anhydrous sodium sulfate and evaporated. The residue was purified by a flash silica gel column and eluted with DCM/MeOH to give 0.28 g C1 (R⁷=p-SF₅-Phenyl, R¹⁰=3-MeO-Phenyl, R⁹=4-(4-Methyl-imidazol-1-yl)), that is

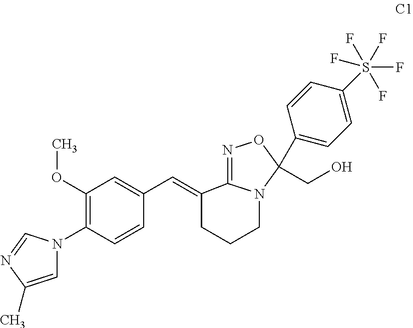

(Retention Time 3.09, Observed mass 557).
   Compound C1 (0.28 g, R⁷=p-SF₅-Phenyl, R¹⁰=3-MeO-Phenyl, R⁹=4-(4-Methyl-imidazol-1-yl)) was resolved by chiral AD column and eluted with Hexanes/Isopropanol to give 73 mg of enantiomer A of C1 (R⁷=p-SF₅-Phenyl, and R¹⁰=3-MeO-Phenyl, R⁹=4-(4-Methyl-imidazol-1-yl)) and 101 mg of enantiomer B of C1 (R⁷=p-F-Phenyl, R¹⁰=3-MeO-Phenyl, R⁹=4-(4-Methyl-imidazol-1-yl)).

If one were to follow procedures similar to those of Examples 1 and 2 then one would obtain compounds D1 to D7 and D9 to D12 below. If one were to follow procedures similar to those of Example 1 then one would obtain compound D8 below.

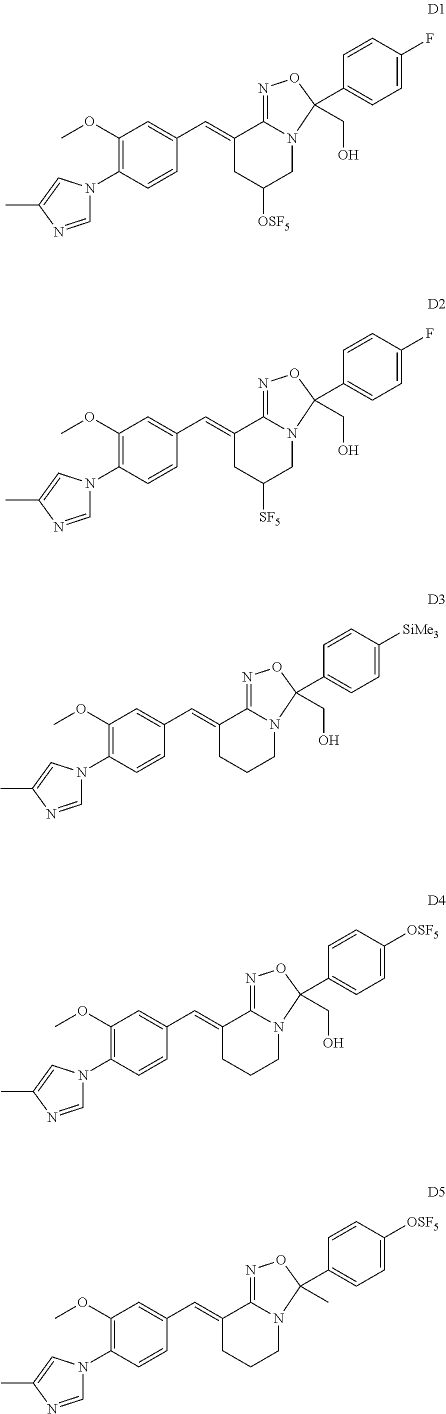

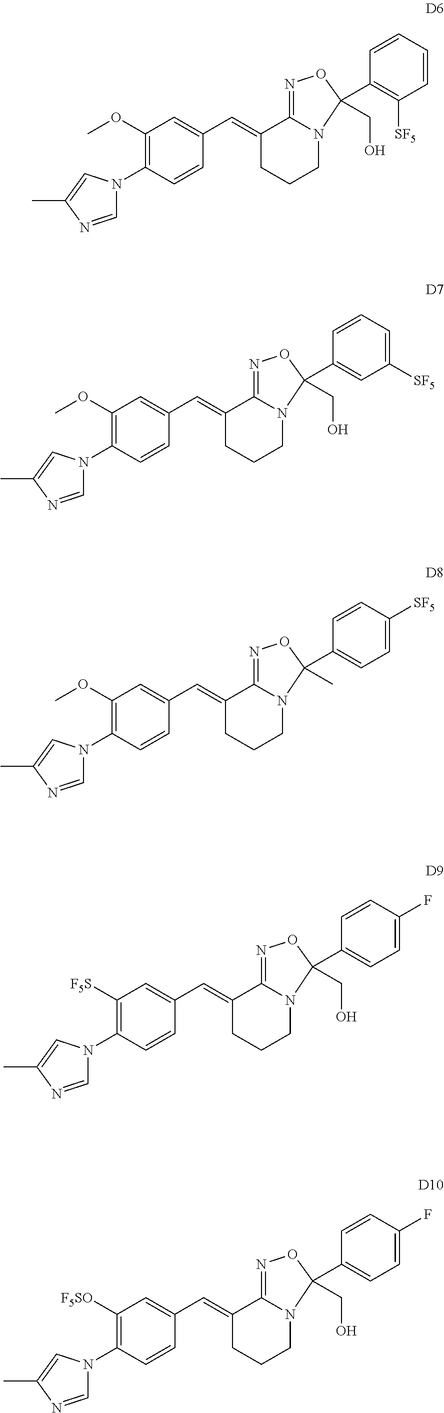

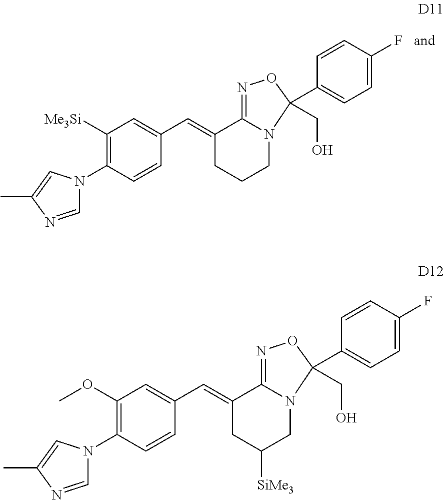

### Assay:

Secretase Reaction and Aβ Analysis in Whole Cells: HEK293 cells overexpressing APP with Swedish and London mutations were treated with the specified compounds for 5 hour at 37° C. in 100 ml of DMEM medium containing 10% fetal bovine serum. At the end of the incubation, total Aβ, Aβ40 and Aβ42 were measured using electrochemiluminescence (ECL) based sandwich immunoassays. Total Aβ was determined using a pair of antibodies TAG-W02 and biotin-4G8, Aβ40 was identified with antibody pairs TAG-G2-10 and biotin-4G8, while Aβ42 was identified with TAG-G2-11 and biotin-4G8. The ECL signal was measured using Sector Imager 2400 (Meso Scale Discovery).

MS Analysis of Aβ Profile: Aβ profile in conditioned media was determined using surface enhanced laser desorption/ionization (SELDI) mass spectrometry. Conditioned media was incubated with antibody W02 coated PS20 ProteinChip array. Mass spectra of Aβ captured on the array were read on SELDI ProteinChip Reader (Bio-Rad) according to manufacture's instructions.

CSF Aβ Analysis: Aβ in rat CSF was determined using MSD technology as described above. Aβ40 was measured using antibody pair Tag-G2-10 and biotin-4G8, while Aβ42 was measured using Tag-anti Aβ42 (Meso Scale Discovery) and biotin-4G8. The ECL signal was measured using Sector Imager 2400 (Meso Scale Discovery).

MS analysis of Aβ profile: To isolate Aβ products from conditioned media, cells expressing APP were grown to 90% confluence and re-fed with fresh media containing γ-secretase modulator. The conditioned media, harvested after 16 h of incubation, were incubated overnight with antibody W02 in RIPA buffer (20 mM Tris-HCl, pH7.4, 150 mM NaCl, 0.2% Twenn 20, 0.2% Triton 100 and 0.2% NP40). Protein A plus G agarose (Calbiochem) was added to the reaction and the mixture was rocked at room temperature for another 2 h. The agarose beads were then collected by centrifugation and washed 3 times with RIPA buffer and twice with 20 mM Tris (pH 7.4). The immunoprecipitated peptides were eluted from the beads with 10 μL of 10% acetonitrile/0.1% trifluoroacetic acid (TFA).

Matrix-assisted laser desorption/ionization mass spectrometric (MALDI MS) analysis of Aβ was performed on a Voyager-DE STR mass spectrometer (ABI, Framingham, Mass.). The instrument is equipped with a pulsed nitrogen laser (337 nm). Mass spectra were acquired in the linear mode with an acceleration voltage of 20 kV. Each spectrum presented in this work represents an average of 256 laser shots. To prepare the sample-matrix solution, 1 μL of immunoprecipitated Aβ sample was mixed with 3 μL of saturated α-cyano-4-hydroxycinnamic acid solution in 0.1% TFA/acetonitrile. The sample-matrix solution was then applied to the sample plate and dried at ambient temperature prior to mass spectrometric analysis. All the spectra were externally calibrated with a mixture of bovine insulin and ACTH (18-39 clip).

Certain compounds of this invention had an Aβ42 IC50 in the range of about 19 nM to about 530 nm, and an Aβtotal/Aβ42 IC50 ratio in the range of about 35 to about 1053.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1-22. (canceled)

23. A compound having the general structure shown in the formula: 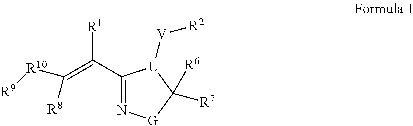
or a pharmaceutically acceptable salt, solvate, or ester thereof, wherein:
U is 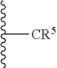
or N;
G is O or S;
V is selected from the group consisting of a bond, O, —C(O)—, and N(R¹⁴);
R¹ is selected from the group consisting of: H, halo, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 substituents independently selected from the group consisting of the R²¹ groups;
R² is selected from the group consisting of: H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 substituents independently selected from the group consisting of the R²¹ groups; or
R¹ and R² are joined together to form a C5-C8 cycloalkyl or a 5-8 membered heterocyclyl moiety, wherein each of said cycloalkyl or heterocyclyl moiety is optionally substituted with 1-5 substituents independently selected from the group consisting of the R²¹ groups; or
R¹ and R⁸ are taken together to form a bond;
R⁵, R⁶ and R⁷ are each independently selected from the group consisting of H, —C(O)R¹⁵, —C(O)OR¹⁵, —C(O)N(R¹⁵)(R¹⁶), —C(═NOR¹⁵)R¹⁶, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, and wherein each of said alkyl-, alkenyl- and alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-5 substituents independently selected from the group consisting of the R²¹ groups; or
R⁶ and R⁷ are joined together to form a carbocyclic spirocyclic moiety or a heterocyclic spirocyclic moiety wherein each of said carbocyclic spirocyclic moiety and heterocyclic spirocyclic moiety is: (i) optionally substituted with 1-4 substituents independently selected from the group consisting of the R²¹ groups, or (ii) fused with an aryl, heteroaryl, cycloalkyl or heterocycloalkyl ring, and wherein each of said carbocyclic spirocyclic moiety, heterocyclic spirocyclic moiety, aryl, heteroaryl, cycloalkyl and heterocycloalkyl ring is optionally substituted with 1-4 substituents independently selected from the group consisting of the R²¹ groups;
R⁸ is selected from the group consisting of H, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R₁₇), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, —N₃, —NO₂ and —S(O)₂R²⁴, alkyl-, aryl-, arylalkyl-, alkylaryl-, cycloalkyl-, cycloalkylalkyl-, heteroaryl-, heteroarylalkyl-, heterocyclyl- and heterocyclyalkyl-, and wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- moieties is optionally substituted with 1-3 substituents independently selected from the group consisting of the R²¹ groups;
R¹⁰ is selected from the group consisting of a bond, alkyl-, aryl-, arylalkyl-, arylalkenyl-, alkylaryl-, cycloalkyl-, cycloalkylalkyl-, heteroaryl-, heteroarylalkyl-, heterocyclyl-, heterocyclyalkyl- and the moieties: 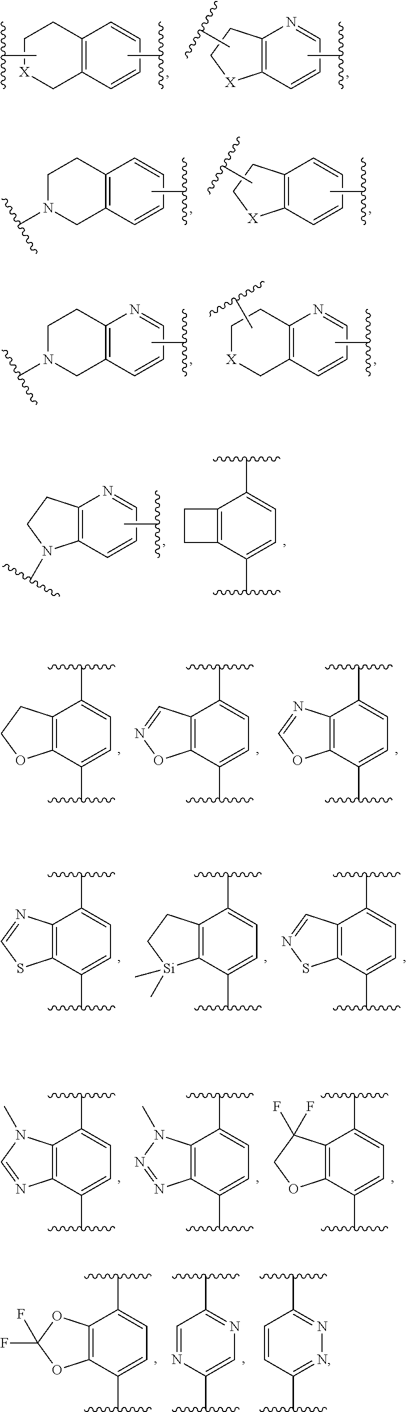 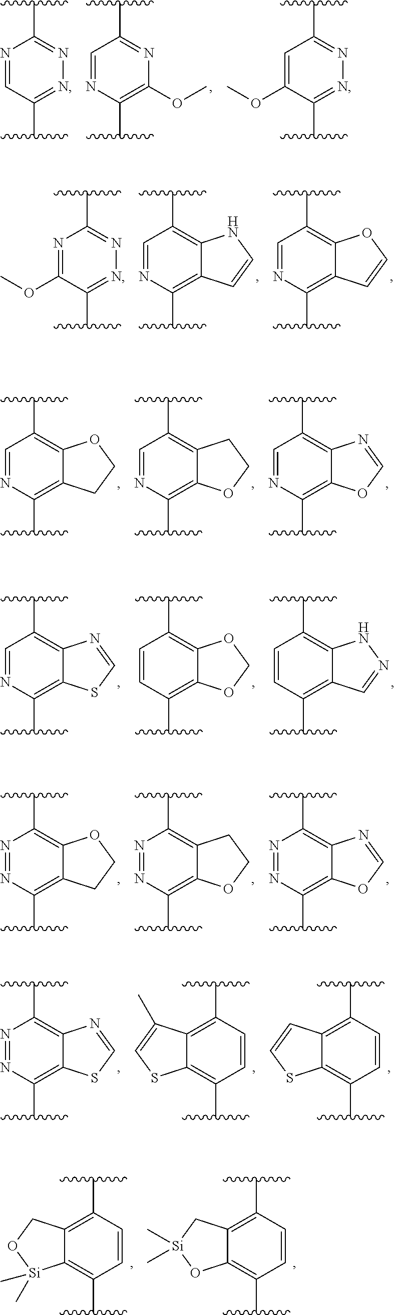 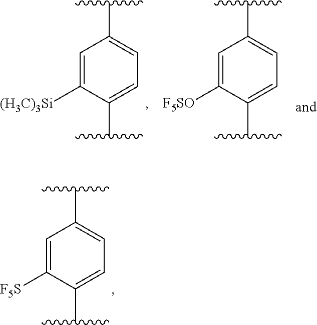
wherein X is selected from the group consisting of: O, N(R¹⁴) or S; and wherein each of said R¹⁰ groups (excluding the bond) is optionally substituted with 1-3 substituents independently selected from the group consisting of the R²¹ groups;
or, alternatively, R⁸ and R¹⁰, together with the carbon atom to which they are bound, can form a C₄-C₇ carbocyclic (e.g., cycloalkyl) ring, or a 4-7 membered heterocyclyl ring, or a C₄-C₇ carbocyclenyl (e.g., cycloalkenyl) ring, or a 4-7 membered heterocyclenyl ring; and wherein said carbocyclic ring, heterocyclyl ring, carbocyclenyl ring, or heterocyclenyl ring is optionally substituted with 1-5 independently selected R²¹ substituents;
R⁹ is selected from the group consisting of H, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, and wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- is optionally substituted with 1-3 substituents independently selected from the group consisting of the R²¹ groups;
R¹⁴ is selected from the group consisting of H, alkyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶), and wherein each of the alkyl, cycloalkyl, cycloalkylalkyl-, cycloalkenyl, heterocyclyl, heterocyclylalkyl-, aryl, arylalkyl-, heteroaryl, heteroarylalkyl- groups is optionally substituted with 1-5 independently selected R²¹ groups;
R¹⁵, R¹⁶ and R¹⁷ are independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ₙ-alkyl-, (R¹⁸)ₙ-cycloalkyl-, (R¹⁸)ₙ-cycloalkylalkyl-, (R¹⁸)ₙ-heterocyclyl-, (R¹⁸)ₙ-heterocyclylalkyl-, (R¹⁸)ₙ-aryl-, (R¹⁸)ₙ-arylalkyl-, (R¹⁸)ₙ-heteroaryl- and (R¹⁸)ₙ-heteroarylalkyl-;
n is 1 to 5;
Each R¹⁸ is independently selected from the group consisting of: alkyl, alkenyl, alkynyl, aryl, arylalkyl-, arylalkenyl-, arylalkynyl-, —NO₂, halo, heteroaryl, —CF₃, —CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl); or
two R¹⁸ moieties on adjacent carbons can be linked together to form: 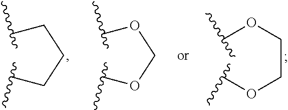
R¹⁹ is selected from the group consisting of: alkyl, cycloalkyl, aryl, arylalkyl and heteroarylalkyl;
R²⁰ is selected from the group consisting of: alkyl, cycloalkyl, aryl, halo substituted aryl, arylalkyl, heteroaryl and heteroarylalkyl;
Each R²¹ is independently selected from the group consisting of: alkyl, alkenyl, alkynyl, cycloalkyl, cycloalkylalkyl-, cycloalkenyl, heterocycloalkyl, heterocycloalkylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SF₅, —OSF₅, —Si(R²⁴)₃ wherein each R²⁴ is independently selected —SR¹⁵, —S(O)NR¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)NR¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, —N₃, —NO₂ and —S(O)₂R²⁴;
wherein each of the alkyl, cycloalkenyl, cycloalkyl, cycloalkylalkyl-, heterocycloalkyl, heterocycloalkylalkyl-, aryl, arylalkyl-, heteroaryl, heteroarylalkyl-, alkenyl and alkynyl R²¹ groups is optionally substituted with 1 to 5 independently selected R²² groups;
Each R²² is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R²⁴)₃ wherein each R²⁴ is independently selected —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, —NO₂, —S(O)R¹⁵ and —S(O)₂R²⁴; and
Each R²⁴ is independently selected from the group consisting of alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl-, (R¹⁸)ₙ-alkyl-, (R¹⁸)ₙ-cycloalkyl-, (R¹⁸)ₙ-cycloalkylalkyl-, (R¹⁸)ₙ-heterocyclyl-, (R¹⁸)ₙ-heterocyclylalkyl-, (R¹⁸)ₙ-aryl-, (R¹⁸)ₙ-arylalkyl-, (R¹⁸)ₙ-heteroaryl- and (R¹⁸)ₙ-heteroarylalkyl- (wherein R¹⁸ and n are as defined above); and
with the proviso that:
(a) there is present at least one group selected from the group consisting of: —SF₅, —OSF₅, and —Si(R²⁴)₃ (wherein each R²⁴ is independently selected), and wherein there is more than one group, each group is independently selected, or
(b) there is present an R¹⁰ group selected from the group consisting of: 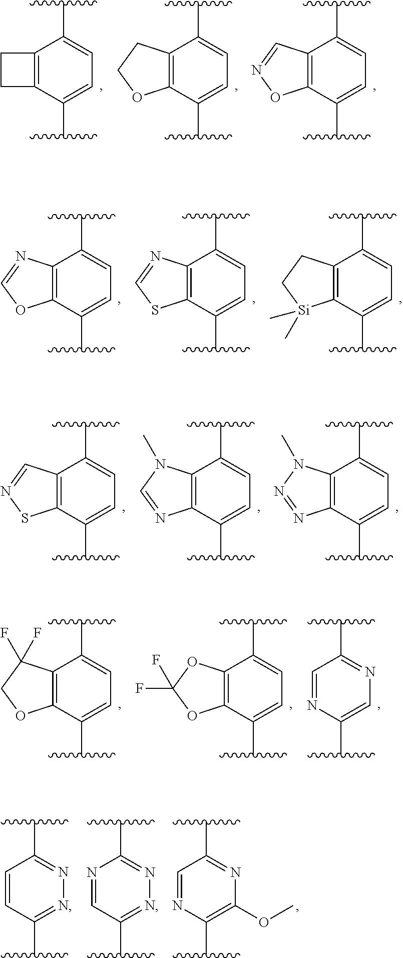 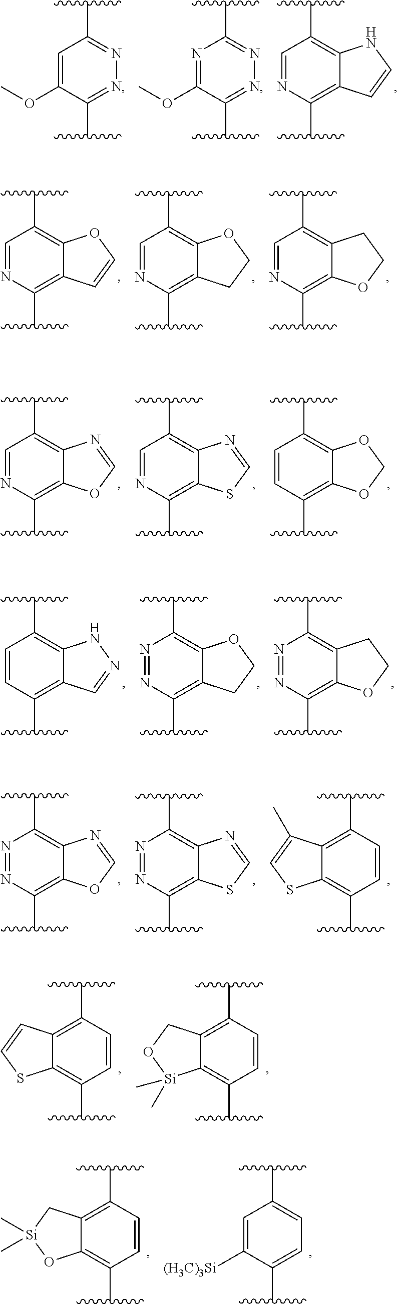 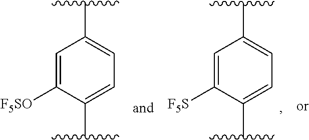
(c) there is present at least one group selected from the group consisting of: —SF₅, —OSF₅, —Si(R²⁴)₃ (wherein each R²⁴ is independently selected), and wherein there is more than one group, each group is independently selected, and there is present an R¹⁰ group selected from the group consisting of:

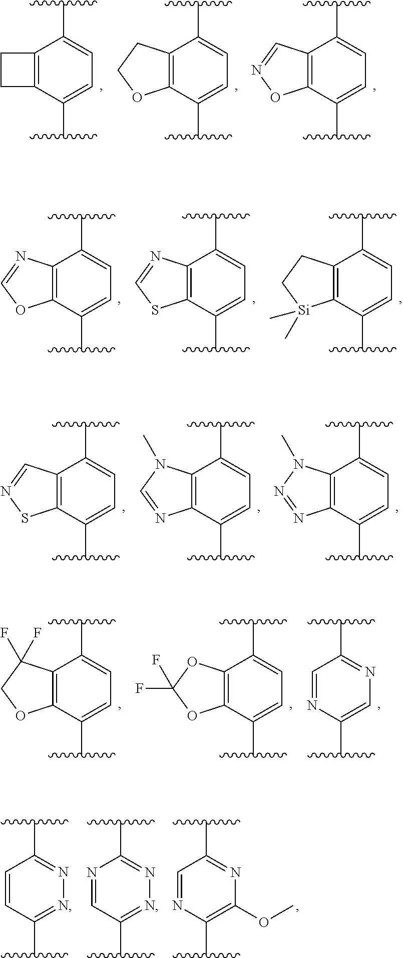

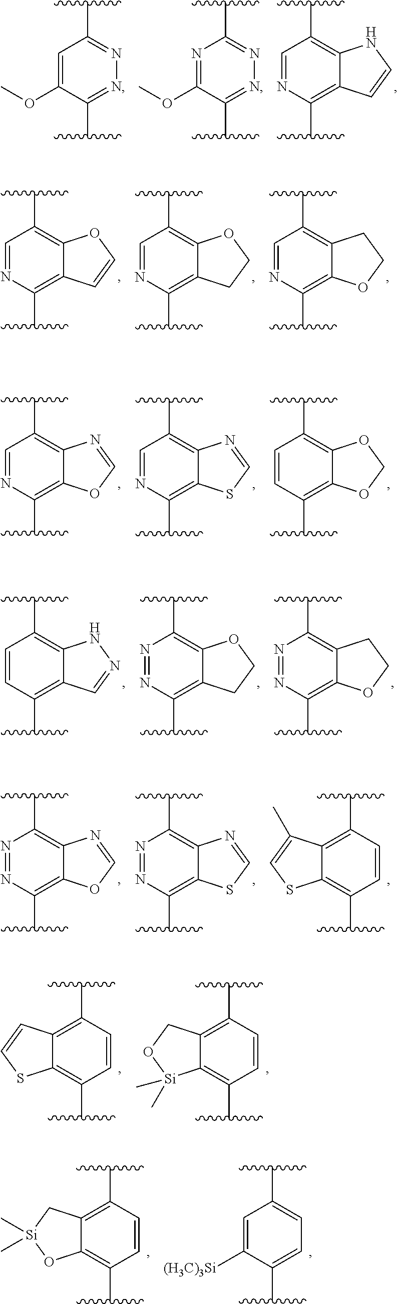

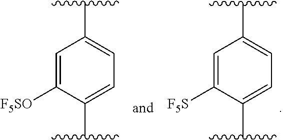

24. The compound of claim 23 wherein:
(a) there is present at least one group selected from the group consisting of: —SF₅, —OSF₅, and —Si(R²⁴)₃ (wherein each R²⁴ is independently selected), and wherein there is more than one group, each group is independently selected; or
(b) there is present at least one group selected from the group consisting of: —SF₅ and —OSF₅, and when there is more than one group, each group is independently selected; or
(c) R¹ and R² are joined together to form a 5 to 8 membered cycloalkyl ring, and said ring is substituted with a group selected from the group consisting of —SF₅, —OSF₅ and —Si(R²⁴)₃; or
(d) R¹ and R² are joined together to form a 5 to 8 membered cycloalkyl ring, and said ring is substituted with a group selected from the group consisting of —SF₅, —OSF₅ and —Si(R²⁴)₃ and said ring is substituted with a group selected from the group consisting of —SF₅ and —OSF₅; or
(e) R¹ and R² are joined together to form a 5 to 8 membered heterocyclyl ring, and said ring is substituted with a group selected from the group consisting of —SF₅, —OSF₅ and —Si(R²⁴)₃; or
(f) R¹ and R² are joined together to form a 5 to 8 membered heterocyclyl ring, and said ring is substituted with a group selected from the group consisting of —SF₅, —OSF₅ and —Si(R²⁴)₃, and said ring is substituted with a group selected from the group consisting of —SF₅ and —OSF₅; or
(g) R⁶ and R⁷ are joined together to form a carbocyclic spirocyclic moiety, and said spirocyclic moiety is substituted with a group selected from the group consisting of —SF₅, —OSF₅ and —Si(R²⁴)₃; or
(h) R⁶ and R⁷ are joined together to form a carbocyclic spirocyclic moiety, and said spirocyclic moiety is substituted with a group selected from the group consisting of —SF₅, —OSF₅ and —Si(R²⁴)₃, and said spirocyclic moiety is substituted with a group selected from the group consisting of —SF₅ and —OSF₅; or
(i) R⁶ and R⁷ are joined together to form a heterocyclic spirocyclic moiety, and said spirocyclic moiety is substituted with a group selected from the group consisting of —SF₅, —OSF₅ and —Si(R²⁴)₃; or
(j) R⁶ and R⁷ are joined together to form a heterocyclic spirocyclic moiety, and said spirocyclic moiety is substituted with a group selected from the group consisting of —SF₅, —OSF₅ and —Si(R²⁴)₃, and said spirocyclic moiety is substituted with a group selected from the group consisting of —SF₅ and —OSF₅.

25. The compound of claim 23 selected from the group consisting of
(a) compounds III to X, wherein said compounds are substituted, and wherein 1 to 3 of the substituents present are selected from the group consisting of —SF₅, —OSF₅ and —Si(R²⁴)₃; or
(b) compounds III to X, wherein said compounds are substituted, and wherein 1 to 3 of the substituents present are selected from the group consisting of —SF₅ and —OSF₅.

26. The compound of claim 23 wherein:
(a) R⁶ is alkyl substituted with 1-5 independently selected R²¹ moieties, and 1 to 3 R²¹ moieties are selected from the group consisting of: —SF₅, —OSF₅ and —Si(R²⁴)₃; or
(b) R⁶ is alkyl substituted with 1-5 independently selected R²¹ moieties, and 1 to 3 R²¹ moieties are selected from the group consisting of: —SF₅ and —OSF₅; or
(c) R⁶ is selected from the group consisting of: H, alkyl, cycloalkyl, —C(O)OR¹⁵, alkyl substituted with 1-3 halos, —C(O)R¹⁵, and alkyl substituted with —OR¹⁵; or
(d) R⁶ is selected from the group consisting of: H, methyl, methyl substituted with —OH, and methyl substituted with OCH₃.

27. The compound of claim 23 wherein
(a) R⁷ is aryl substituted with 1 to 3 independently selected R²¹ moieties wherein at least one R²¹ moiety is selected from the group consisting of —SF₅, —OSF₅ and —Si(R²⁴)₃; or
(b) R⁷ is aryl substituted with 1 to 3 independently selected R²¹ moieties wherein at least one R²¹ moiety is selected from the group consisting of —SF₅, —OSF₅ and —Si(R²⁴)₃, and said aryl is phenyl; or
(c) R⁷ is aryl substituted with 1 to 3 independently selected R²¹ moieties wherein at least one R²¹ moiety is selected from the group consisting of —SF₅ and —OSF₅; or
(d) R⁷ is aryl substituted with 1 to 3 independently selected R²¹ moieties wherein at least one R²¹ moiety is selected from the group consisting of —SF₅ and —OSF₅, and aryl is phenyl; or
(e) R⁷ is phenyl substituted with 1-3 substituents independently selected from the group consisting of halo, alkyl, —CN, —NH₂, —NH(alkyl), —N(alkyl)₂, hydroxy, alkoxy, aryl, heteroaryl, —SF₅, —OSF₅ and —Si(R²⁴)₃, and wherein at least one R²¹ moiety is selected from the group consisting of —SF₅, —OSF₅ and —Si(R²⁴)₃; or
(f) R⁷ is phenyl substituted with 1-3 substituents independently selected from the group consisting of halo, alkyl, —CN, —NH₂, —NH(alkyl), —N(alkyl)₂, hydroxy, alkoxy, aryl, heteroaryl, —SF₅, —OSF₅ and —Si(R²⁴)₃, and wherein at least one R²¹ moiety is selected from the group consisting of —SF₅ and —OSF₅; or
(g) R⁷ is naphthyl substituted with 1-4 R²¹ substituents independently selected from the group consisting of halo, alkyl, —CN, —NH₂, —NH(alkyl), —N(alkyl)₂, hydroxy, alkoxy, aryl, heteroaryl, —SF₅, —OSF₅ and —Si(R²⁴)₃, and wherein at least one R²¹ group is selected from the group consisting of —SF₅, —OSF₅ and —Si(R²⁴)₃; or
(h) R⁷ is biphenyl substituted with 1-4 R²¹ substituents independently selected from the group consisting of halo, alkyl, —CN, —NH₂, —NH(alkyl), —N(alkyl)₂, hydroxyl, alkoxy, —SF₅, —OSF₅ and —Si(R²⁴)₃, and wherein at least one R²¹ group is selected from the group consisting of —SF₅, —OSF₅ and —Si(R²⁴)₃; or
(i) R⁷ is selected from the group consisting of: (1) aryl substituted with 1-3 R²¹ moieties, (2) aryl substituted with —OR¹⁵ wherein R¹⁵ is (i) an alkyl substituted with 1-3 halos, or (ii) alkyl, (3) aryl, (4) aryl substituted with alkyl wherein said alkyl is substituted with 1-3 halos, (5) aryl substituted with aryl, (6) alkyl, (7) heteroaryl, (8) arylalkyl-, and (9) cycloalkyl); or
(j) R⁷ is aryl; or
(k) R⁷ is an unsubstituted phenyl; or
(l) R⁷ is a phenyl which is substituted with 1-4 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups; or
(m) R⁷ is unsubstituted naphthyl; or
(n) R⁷ is naphthyl which is substituted with 1-4 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups; or
(o) R⁷ is unsubstituted biphenyl; or
(p) R⁷ is biphenyl which is substituted with 1-4 substituents which can be the same or different, each substituent being independently selected from the group consisting of halo, alkyl, —CN, —NH₂, —NH(alkyl), —N(alkyl)₂, hydroxy and alkoxy groups.

28. The compound of claim 23 wherein:
(a) R⁶ is alkyl, and R⁷ is phenyl substituted with 1-3 R²¹ substituents independently selected from the group consisting of halo, alkyl, —CN, —NH₂, —NH(alkyl), —N(alkyl)₂, hydroxy, alkoxy, aryl, heteroaryl, halo, alkyl, —CN, —NH₂, —NH(alkyl), —N(alkyl)₂, hydroxyl, alkoxy, SF₅, —OSF₅ and —Si(R²⁴), and wherein at least one R²¹ group is selected from the group consisting of —SF₅, —OSF₅ and —Si(R²⁴)₃; or
(b) R⁶ is alkyl substituted with 1-5 independently selected R²¹ moieties, and R⁷ is phenyl substituted with 1-3 R²¹ substituents independently selected from the group consisting of halo, alkyl, —CN, —NH₂, —NH(alkyl), —N(alkyl)₂, hydroxy, alkoxy, aryl, heteroaryl, halo, alkyl, —CN, —NH₂, —NH(alkyl), —N(alkyl)₂, hydroxyl, alkoxy, SF₅, —OSF₅ and —Si(R²⁴)₃, and wherein at least one R²¹ group is selected from the group consisting of —SF₅, —OSF₅ and —Si(R²⁴)₃; or
(c) R⁶ is alkyl substituted with 1-5 independently selected R²¹ moieties, and R⁷ is phenyl substituted with 1-3 R²¹ substituents independently selected from the group consisting of halo, alkyl, —CN, —NH₂, —NH(alkyl), —N(alkyl)₂, hydroxy, alkoxy, aryl, heteroaryl, halo, alkyl, —CN, —NH₂, —NH(alkyl), —N(alkyl)₂, hydroxyl, alkoxy, —SF₅ and —OSF₅, wherein at least one R²¹ group on said phenyl is selected from the group consisting of —SF₅ and —OSF₅; or
(d) R⁶ is H and R⁷ is a biphenyl which can be unsubstituted or optionally independently substituted with 1-4 substituents which can be the same or different, each substituent being independently selected from the group consisting of halo, alkyl, —CN, —NH₂, —NH(alkyl), —N(alkyl)₂, hydroxy and alkoxy groups, or
(e) wherein R⁶ is methyl, and R⁷ is a biphenyl which can be unsubstituted or optionally independently substituted with 1-4 substituents which can be the same or different, each substituent being independently selected from the group consisting of halo, alkyl, —CN, —NH₂, —NH(alkyl), —N(alkyl)₂, hydroxy and alkoxy groups, or
(f) R⁶ is H, and R⁷ is a phenyl which can be unsubstituted or optionally independently substituted with 1-4 substituents which can be the same or different, each substituent being independently selected from the group consisting of halo, alkyl, —CN, —NH₂, —NH(alkyl), —N(alkyl)₂, hydroxy and alkoxy groups, or
(g) R⁶ is methyl, and R⁷ is a biphenyl which can be unsubstituted or optionally independently substituted with 1-4 substituents which can be the same or different, each substituent being independently selected from the group consisting of halo, alkyl, —CN, —NH₂, —NH(alkyl), —N(alkyl)₂, hydroxy and alkoxy groups, or
(h) R⁶ is alkyl substituted with 1-5 independently selected R²¹ moieties, and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups, or
(i) R⁶ is alkyl substituted with one R²¹ moiety, and R⁷ is phenyl substituted with 1-3 substituents which can be the same or different, each substituent being independently selected from the group consisting of halo, alkyl, —CN, —NH₂, —NH(alkyl), —N(alkyl)₂, hydroxy, alkoxy, aryl and heteroaryl groups,
(j) R⁶ and R⁷ are ioined to form the suirocvclic unit: 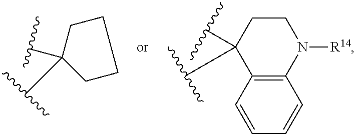
or
(h) R⁶ and R⁷ are joined to form the spirocyclic unit:

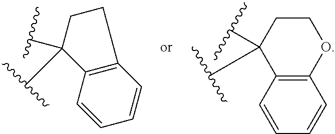

29. The compound of claim 23 wherein:
(a) R⁹ is selected from the group consisting of 1g to 13g and R¹⁰ is selected from the group consisting of: 1A to 42A; or
(b) the R⁹—R¹⁰— moiety is selected from the group consisting of: 1b to 50b; or
(c) the R⁹—R¹⁰— moiety is 50b.

30. The compound of claim 23, wherein:
(a) R² is selected from the group consisting of H, alkyl, alkoxyalkyl-, (4-alkoxy)phenylmethyl-, and arylalkyl-; or
(b) R² is selected from the group consisting of: methyl, 3-methoxypropyl-, phenylmethyl-, and (4-methoxy)phenylmethyl-; or
(c) R² is selected from the group consisting of: H, —(CH₂)₃OCH₃, —CH₃, —CH₂CH₃, —(CH₂)₂OCH₃, —(CH₂)₂CH(CH₃)₂, —CH₂CH(CH₃)CH₂CH₃, —CH₂CH₂CH₃,

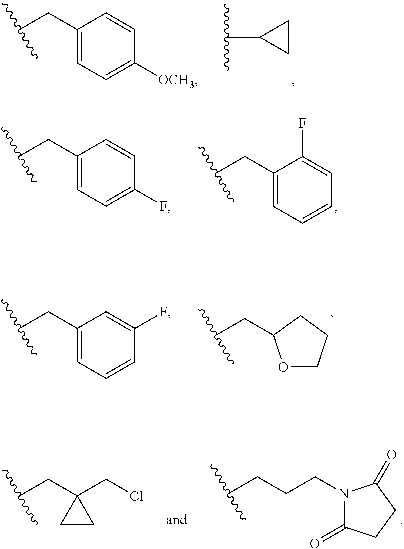

31. The compound of claim 23, wherein:
(a) U is N; or
(b) U is N and R² is 3-methoxypropyl-.

32. The compound of claim 23, wherein:
(a) R¹ is selected from the group consisting of: H and alkyl; or
(b) R¹ is selected from the group consisting of: H and methyl; or
(c) R¹ is methyl; or
(d) R¹ and R² are joined together to form a cyclopentyl ring, which is unsubstituted, or (b) R¹ and R² are joined together to form a cyclopentyl ring, which is substituted with 1-3 substituents which can be the same or different, each being independently selected from the group consisting of halo, alkyl, —CN, —NH₂, —NH(alkyl), —N(alkyl)₂, hydroxy and alkoxy groups, or (c) R¹ and R² are joined together to form a cyclohexyl ring, which is unsubstituted, or (d) R¹ and R² are joined together to form a cyclohexyl ring, which is substituted with 1-3 substituents which can be the same or different, each being independently selected from the group consisting of halo, alkyl, —CN, —NH₂, —NH(alkyl), —N(alkyl)₂, hydroxy and alkoxy groups.

33. The compound of claim 23, wherein:
(a) U is N, and R¹ and R² are joined together to form a piperidinyl ring including the N of U as the nitrogen of said piperidinyl ring, which is unsubstituted; or
(b) U is N, and R¹ and R² are joined together to form a piperidinyl ring including the N of U as the nitrogen of said piperidinyl ring, wherein said piperidinyl ring is substituted with 1-3 substituents which can be the same or different, each being independently selected from the group consisting of halo, alkyl, —CN, —NH₂, —NH(alkyl), —N(alkyl)₂, hydroxy and alkoxy groups; or
(c) U is N, and R¹ and R² are joined together to form a pyrrolidinyl ring including the N of U as the nitrogen of said pyrrolidinyl ring, which is unsubstituted; or
(d) U is N, and R¹ and R² are joined together to form a pyrrolidinyl ring including the N of U as the nitrogen of said pyrrolidinyl ring, wherein said pyrrolidinyl ring is substituted with 1-3 substituents which can be the same or different, each being independently selected from the group consisting of halo, alkyl, —CN, —NH₂, —NH(alkyl), —N(alkyl)₂, hydroxy and alkoxy groups; or
(e) U is N, and R¹ and R² are joined together to form a piperazinyl ring including the N of U as a nitrogen of said piperazinyl ring, which is unsubstituted; or
(f) U is N, and R¹ and R² are joined together to form a piperazinyl ring including the N of U as a nitrogen of said piperazinyl ring, wherein said piperazinyl ring is substituted with 1-3 substituents which can be the same or different, each being independently selected from the group consisting of halo, alkyl, —CN, —NH₂, —NH(alkyl), —N(alkyl)₂, hydroxy and alkoxy groups.

34. The compound of claim 23, wherein: (a) R⁸ is H; or (b) R⁸ is alkyl; or R⁸ is methyl.

35. The compound of claim 23, wherein:
(a) R¹⁰ is aryl; or
(b) R¹⁰ is aryl substituted with 1 to 3 independently selected R²¹ moieties; or
(c) R¹⁰ is aryl substituted with 1 to 3 R²¹ moieties, wherein each R²¹ moiety is the same or different —OR¹⁵ group; or
(d) R¹⁰ is aryl substituted with 1 R²¹ moiety; or
(e) R¹⁰ is aryl substituted with one R²¹ moiety, and said R²¹ moiety is —OR¹⁵; or
(f) R¹⁰ is aryl substituted with one R²¹ moiety, said R²¹ moiety is —OR¹⁵, and said R¹⁵ is alkyl; or
(g) R¹⁰ is aryl substituted with one R²¹ moiety, said R²¹ moiety is —OR¹⁵, said R¹⁵ is alkyl, and said alkyl is methyl; or
(h) R¹⁰ is phenyl; or
(i) R¹⁰ is phenyl substituted with 1 to 3 independently selected R²¹ moieties; or
(j) R¹⁰ is phenyl substituted with 1 to 3 R²¹ moieties, wherein each R²¹ moiety is the same or different —OR¹⁵ group; or
(k) R¹⁰ is phenyl substituted with 1 R²¹ moiety; or
(l) R¹⁰ is phenyl substituted with one R²¹ moiety, and said R²¹ moiety is —OR¹⁵; or
(m) R¹⁰ is phenyl substituted with one R²¹ moiety, said R²¹ moiety is —OR¹⁵, and said R¹⁵ is alkyl, or
(n) R¹⁰ is phenyl substituted with one R²¹ moiety, said R²¹ moiety is —OR¹⁵, said R¹⁵ is alkyl, and said alkyl is methyl; or
(o) R¹⁰ is heteroaryl; or
(p) R¹⁰ is selected from the group consisting of:

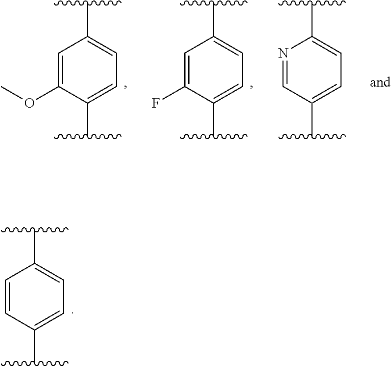

36. The compound of claim 23, wherein: (a) R⁹ is unsubstituted heteroaryl, or (b) R⁹ is heteroaryl which is substituted with 1-3 substituents which can be the same or different, each substituent being independently selected from the group consisting of halo, alkyl, CN, NH₂, NH(alkyl), N(alkyl)₂, hydroxy and alkoxy groups, or (c) R⁹ is imidazol-1-yl, or (d) R⁹ is 4-methyl-imidazol-1-yl, or (e) R⁹ is 5-chloro-4-methyl-imidazol-1-yl.

37. The compound of claim 23 wherein:
(a) the —R¹⁰—R⁹ moiety is: 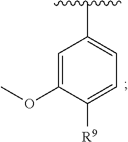
or
(b) the —R¹⁰—R⁹ moiety is: 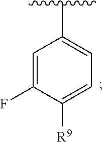
or
(c) the —R¹⁰—R⁹ moiety is: 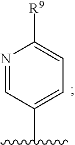
or
(d) the R⁹—R¹⁰— moiety is: 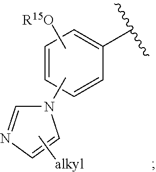
or
(e) the R⁹—R¹⁰— moiety is: 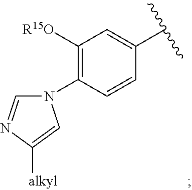
or
(f) the R⁹—R¹⁰— moiety is: 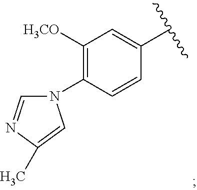
or
(g) the R⁹—R¹⁰— moiety is: 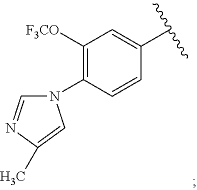
or
(h) the R⁹—R¹⁰— moiety is: 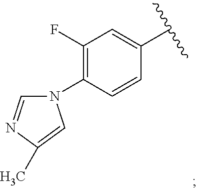
or
(i) the R⁹—R¹⁰— moiety is: 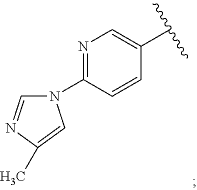
or
(j) the R⁹—R¹⁰— moiety is:

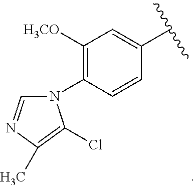

38. A compound selected from the group consisting of: compounds B7, C1, and D1 to D12.

39. A pharmaceutical composition comprising a therapeutically effective amount of at least one compound of claim 23, or a pharmaceutically acceptable salt thereof, and at least one pharmaceutically acceptable carrier.

40. A method of modulating gamma-secretase comprising administering an effective amount of one or more compounds of claim 23 or a pharmaceutically acceptable salt thereof to a patient in need of treatment.

41. A method of inhibiting the deposition of amyloid protein in, on or around neurological tissue, comprising administering an effective amount of one or more compounds of claim 23 or a pharmaceutically acceptable salt thereof to a patient in need of treatment.

42. A method of treating Alzheimer's disease, comprising administering an effective amount of one or more compounds of claim 23 or a pharmaceutically acceptable salt thereof to a patient in need of treatment.
